(19) United States
(12) Patent Application Publication
(10) Pub. No.: US 2023/0130791 A1
(43) Pub. Date: Apr. 27, 2023

(54) A Combined Inhibition of EGFR and NRF2 in The Treatment of Malignant Glioma

(71) Applicant: United States Government as represented by the Department of Veterans Affairs, Washington, DC (US)

(72) Inventor: Amyn A. Habib, Dallas, TX (US)

(21) Appl. No.: 17/910,637

(22) PCT Filed: Mar. 10, 2021
(86) PCT No.: PCT/US2021/021777

§ 371 Date: Sep. 9, 2022

Related U.S. Application Data
(60) Provisional application No. 62/988,250, filed on Mar. 11, 2020.

(51) Int. Cl. A61K 31/517; A61K 39/395; A61K 31/4409; A61K 31/44; A61K 31/427; A61K 31/655; A61P 35/00; A61P 25/00

(52) U.S. Cl. CPC A61K 31/517; A61K 39/3955; A61K 31/4409; A61K 31/44; A61K 31/427; A61K 31/655; A61P 35/00; A61P 25/00; A61K 2039/545

(57) ABSTRACT

The present disclosure is concerned with modulators of EGFR and modulators of Nrf2 for treating various cancers such as, for example, sarcomas, carcinomas, hematological cancers, solid tumors, breast cancer, cervical cancer, gastrointestinal cancer, colorectal cancer, brain cancer, skin cancer, prostate cancer, ovarian cancer, bladder cancer, thyroid cancer, testicular cancer, pancreatic cancer, endometrial cancer, melanomas, gliomas, leukemias, lymphomas, chronic myeloproliferative disorders, myelodysplastic syndromes, myeloproliferative neoplasms, and plasma cell neoplasms (myelomas). This abstract is intended as a scanning tool for purposes of searching in the particular art and is not intended to be limiting of the present invention.

### CROSS-REFERENCE TO RELATED APPLICATIONS

This application claims the benefit of U.S. Application No. 62/988,250, filed on Mar. 11, 2020, the contents of which are hereby incorporated by reference in its entirety.

### STATEMENT REGARDING FEDERALLY SPONSORED RESEARCH

This invention was made with government support under grant number 1R01CA244212-01A1, awarded by the National Institutes of Health. The government has certain rights in the invention.

### BACKGROUND

No targeted treatment is effective in glioblastoma (GBM). Immunotherapy has also, thus far, failed. The efficacy of targeted treatment depends, in part, on the frequency of expression of the target and its oncogenic role. In breast cancer, the official FDA-approved criteria state that if >10% of the tumor cells express estrogen receptors or Her2, treatment with tamoxifen or Herceptin is recommended, while for lung and colon cancer expression of EGFR in just 1% of tumor cells has been used as an indication for EGFR inhibitors. Amplification and mutation of the EGFR gene occurs in 40-50% of GBM patients. EGFRvIII is the most common EGFR mutant found in GBM, and is constitutively active and oncogenic. EGFR wild type (EGFRwt) also plays an oncogenic role in GBM and is a transforming oncogene, and may be activated by ligand or signal constitutively when overexpressed. There is substantial evidence that EGFRwt and EGFRvIII activate each other. EGFRwt is expressed diffusely in the majority of GBMs and in most tumor cells within a GBM, while EGFRvIII expression may be focal. EGFR expression has been detected in up to 81% of GBMs suggesting that EGFR signaling is not limited to the classical subtype of GBM.

Although there is compelling evidence that EGFRvIII and EGFRwt are oncogenic drivers in GBM, EGFR inhibition has not been effective in improving the overall survival of GBM patients or in eliminating EGFR expressing cells from treated tumors. Thus, there is a primary resistance to EGFR inhibition in GBM. Accordingly, there remains a need for compositions and methods for treating gliomas that express EGFRwt and/or that are resistant to EGFR inhibition. These needs and others are met by the present invention.

### SUMMARY

In accordance with the purpose(s) of the invention, as embodied and broadly described herein, the invention, in one aspect, relates to compounds and compositions for use in the prevention and treatment of gliomas such as, for example, malignant gliomas.

Thus, disclosed are methods for treating glioma in a subject, the method comprising administering to the subject an effective amount of an agent that modulates epidermal growth factor receptor (EGFR) signaling, or a pharmaceutically acceptable salt thereof, and an agent that modulates Nrf2 signaling, or a pharmaceutically acceptable salt thereof.

Also disclosed are methods for treating glioma in a patient in need thereof, said method comprising administering to said patient an effective amount of erlotinib and isoniazid.

Also disclosed are pharmaceutical compositions comprising: (a) an agent that modulates EGFR signaling, or a pharmaceutically acceptable salt thereof, (b) an agent that modulates Nrf2 signaling, or a pharmaceutically acceptable salt thereof; and (c) a pharmaceutically acceptable carrier, wherein at least one of the agent that modulates EGFR signaling and the agent that modulates Nrf2 signaling is present in an effective amount.

Also disclosed are methods for making a pharmaceutical composition, the method comprising combining: (a) an agent that modulates EGFR signaling, or a pharmaceutically acceptable salt thereof, (b) an agent that modulates Nrf2 signaling, or a pharmaceutically acceptable salt thereof; and (c) a pharmaceutically acceptable carrier, wherein at least one of the agent that modulates EGFR signaling and the agent that modulates Nrf2 signaling is present in an effective amount.

Also disclosed are kits comprising an agent that modulates EGFR signaling, or a pharmaceutically acceptable salt thereof, and an agent that modulates Nrf2 signaling, or a pharmaceutically acceptable salt thereof, and one or more of: (a) an agent associated with the treatment of cancer; (b) instructions for administering the agent that modulates EGFR signaling and/or the agent that modulates Nrf2 signaling in connection with treating glioma; and (c) instructions for treating cancer.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several aspects and together with the description serve to explain the principles of the invention.

FIG. 1 shows a representative Western blot showing EGFR expression in Mayo PDX lines.

FIG. 2 shows representative data illustrating that GBM 12 cells were treated with erlotinib (1 μM) for 24 hours. 3635 genes were upregulated by RNAseq. The volcano plot shows the differential expression of 17022 genes with counts over 10 in RNAseq analyzed by R. Volcano plot was drawn by Graphpad.

FIG. 3 shows a representative heat map illustrating 40 indicated NRF2 target genes that were induced by erlotinib and could not be blocked by the TNF inhibitor etanercept (Enbrel). Genes with upregulation greater than 1.5 folds and at least 10 counts with erlotinib are shown. The heat map shows the log 2 fold changes, and was drawn by GraphPad Prism 7.0. Enbrel blocks upregulation of 25% of erlotinib induced genes.

FIG. 4A-J shows representative data illustrating that Nrf2 regulated genes are upregulated in multiple May PDX explant cultures in response to EGFR inhibition. Specifically, FIG. 4A shows that erlotinib (1 μM, 24 h) induces Nrf2 target genes (xCT/SLC7A11, GCLM, NQO1) in GBM12 cells. FIG. 4B-D show that SLC7A11, NQO1, and GCLM are induced by erlotinib in multiple PDX lines. FIG. 4E shows an increase in xCT/SLC7A11 protein in response to erlotinib. FIG. 4F shows increase nuclear localization of Nrf2 in GBM12 and GBM6 in response to erlotinib. FIG. 4G shows increased erlotinib-induced activity of an Nrf2 luciferase reporter (ARE-NanoLuc reporter). FIG. 4H shows that erlotinib induced expression of Nrf2 target genes is blocked by Nrf2 inhibitors ML385 (1 μM) and INH (1 μM). FIG. 4I shows that erlotinib induced activation of an Nrf2 reporter is blocked by Nrf2 inhibitors ML385 and INH. FIG. 4J shows that erlotinib induces upregulation of NQO1 protein.

FIG. 5A-N show representative data illustrating that selective inhibition of Nrf2 or downstream targets sensitize cells to erlotinib treatment. Specifically, data for GBM12 (FIG. 5A), GBM6 (FIG. 5B), GBM22 (FIG. 5C), and GBM39 (FIG. 5D) cells is shown. FIG. 5K-L show representative data illustrating that NQO1 knockdown sensitize cells to erlotinib treatment. FIG. 5M-N show representative data illustrating that GCLM knockdown sensitizes cells to erlotinib treatment.

FIG. 6A-E show representative data illustrating that combined treatment of erlotinib plus INH prolonged survival in a GBM6 orthotopic model (n=6 per group). Referring to FIG. 6A, one week after intracranial injection, mice were divided into five groups: vehicle, erlotinib, INH, TMZ, erlotinib+INH (n=6 mice per group). Comparison was made between combination group and erlotonib; P=0.046. FIG. 6B shows representative bioluminescence images from erlotinib and erlotinib plus INH groups at days 1, 15, and 30 after treatment. FIG. 6C shows data illustrating that combined treatment of erlotinib plus INH prolonged survival in a GBM12 orthotopic model (n=4 per group). FIG. 6D shows data illustrating that combined treatment of erlotonib and INH prolonged survival and suppressed tumor growth in an immunocompetent model of glioma. Seven days after intracranial injection of GL261EGFRwt-luc cells into C57BL/6 mice, the mice were divided into 4 groups: vehicle, erlotinib (50 mg/kg), INH (25 mg/kg), and erlotinib plus INH (n=4). Comparison was made between combination group and erlotinib group; p-0.04. FIG. 6E shows representative bioluminescence images from erlotonib and erlotinib plus INH groups at days post treatment as indicated 1, 6, and 12 after treatment.

FIG. 7A-D show representative data illustrating the effect if siRNA knockdown of Nrf2. Specifically, cells were transiently transected with NRF2 wt plasmid (FIG. 7A and FIG. 7B). After 48 h NRF2 target genes NQO1 and GCLM were tested using qPCR. NRF2 overexpression inhibits erlotinib induced cell death (FIG. 7C and FIG. 7D). GBM12 or GBM6 cells were transfected with NRF2 wt plasmid for 24 hours, followed by 20 μM erlotonib treatment for 72 hours. Cell viability was determined by AlamarBlue assay. WB showing overexpression of Nrf2 is shown in FIG. 7E.

FIG. 8 shows representative data illustrating that key Nrf2 components are broadly expressed in GBMs.

FIG. 9A-D show representative data illustrating that Nrf2 is activated in GBM tissue from EGFR TKI treated. Specifically, FIG. 9A shows that Nrf2 immunostaining exhibited more nuclear staining in GBM tissues from afatinib treated patients. Counterstained with hematoxylin. Scale bar=25 μM. FIG. 9B shows quantification of NRF2 nuclear positivity. FIG. 9C and FIG. 9D show that Xct and NQO1 mRNA levels in archival FFPE human glioblastoma from untreated or afatinib treated patients. RNA was extracted from FFPE human glioblastoma tissues with or without afatinib treatment (n=4 per group). Data represents median±IQR, *p<0.05 by Kolmogorov-Smirnov test.

FIG. 10A-D show representative data illustrating that Nrf2 target gene signature predicts prognosis in patients with GBM.

FIG. 11 shows representative data illustrating that there is an increase in ROS following EGFR inhibition in glioma cells.

FIG. 12 shows representative data illustrating that p21 is not unregulated in response to erlotinib, and that p53 is neither induced nor phosphorylated in response to erlotinib.

FIG. 13 shows representative data illustrating that the level of the Nrf2 target gene xCT/SLC7A11 can be used to predict responsiveness to EGFR+Nrf2 inhibition in cell survival assays.

FIG. 14A and FIG. 14B show representative data illustrating that ethionamide is a known Nrf2 inhibitor and preliminary synergizes with EGFR inhibition.

FIG. 15 shows a schematic illustration of the mechanism of EGFR TKI-induced Nrf2 activation.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. DEFINITIONS

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

As used in the specification and in the claims, the term “comprising” can include the aspects “consisting of” and “consisting essentially of.”

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, “IC₅₀” is intended to refer to the concentration of a substance (e.g., a compound or a drug) that is required for 50% inhibition of a biological process, or component of a process, including a protein, subunit, organelle, ribonucleoprotein, etc. In one aspect, an IC₅₀ can refer to the concentration of a substance that is required for 50% inhibition in vivo, as further defined elsewhere herein. In a further aspect, IC₅₀ refers to the half-maximal (50%) inhibitory concentration (IC) of a substance.

As used herein, “EC₅₀” is intended to refer to the concentration of a substance (e.g., a compound or a drug) that is required for 50% agonism of a biological process, or component of a process, including a protein, subunit, organelle, ribonucleoprotein, etc. In one aspect, an EC₅₀ can refer to the concentration of a substance that is required for 50% agonism in vivo, as further defined elsewhere herein. In a further aspect, EC₅₀ refers to the concentration of agonist that provokes a response halfway between the baseline and maximum response.

As used herein, the terms “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “dosage form” means a pharmacologically active material in a medium, carrier, vehicle, or device suitable for administration to a subject. A dosage forms can comprise inventive a disclosed compound, a product of a disclosed method of making, or a salt, solvate, or polymorph thereof, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene 9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed compound, a product of a disclosed method of making, or a salt, solvate, or polymorph thereof, suspended in sterile saline solution for injection together with a preservative.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14^{th} edition), the Physicians' Desk Reference (64^{th} edition), and The Pharmacological Basis of Therapeutics (12^{th} edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; anti-cancer and anti-neoplastic agents such as kinase inhibitors, poly ADP ribose polymerase (PARP) inhibitors and other DNA damage response modifiers, epigenetic agents such as bromodomain and extra-terminal (BET) inhibitors, histone deacetylase (HDAc) inhibitors, iron chelotors and other ribonucleotides reductase inhibitors, proteasome inhibitors and Nedd8-activating enzyme (NAE) inhibitors, mammalian target of rapamycin (mTOR) inhibitors, traditional cytotoxic agents such as paclitaxel, dox, irinotecan, and platinum compounds, immune checkpoint blockade agents such as cytotoxic T lymphocyte antigen-4 (CTLA-4) monoclonal antibody (mAB), programmed cell death protein 1 (PD-1)/programmed cell death-ligand 1 (PD-L1) mAB, cluster of differentiation 47 (CD47) mAB, toll-like receptor (TLR) agonists and other immune modifiers, cell therapeutics such as chimeric antigen receptor T-cell (CAR-T)/chimeric antigen receptor natural killer (CAR-NK) cells, and proteins such as interferons (IFNs), interleukins (ILs), and mAbs; anti-ALS agents such as entry inhibitors, fusion inhibitors, non-nucleoside reverse transcriptase inhibitors (NNRTIs), nucleoside reverse transcriptase inhibitors (NRTIs), nucleotide reverse transcriptase inhibitors, NCP7 inhibitors, protease inhibitors, and integrase inhibitors; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term “therapeutic agent” also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Strem Chemicals (Newburyport, Mass.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and supplemental volumes (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compounds and compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. PHARMACEUTICAL COMPOSITIONS

In one aspect, disclosed are pharmaceutical compositions comprising: (a) an agent that modulates EGFR signaling, or a pharmaceutically acceptable salt thereof; (b) an agent that modulates Nrf2 signaling, or a pharmaceutically acceptable salt thereof; and (c) a pharmaceutically acceptable carrier, wherein at least one of the agent that modulates EGFR signaling and the agent that modulates Nrf2 signaling is present in an effective amount.

In various aspects, the compounds and compositions of the invention can be administered in pharmaceutical compositions, which are formulated according to the intended method of administration. The compounds and compositions described herein can be formulated in a conventional manner using one or more physiologically acceptable carriers or excipients. For example, a pharmaceutical composition can be formulated for local or systemic administration, intravenous, topical, or oral administration.

The nature of the pharmaceutical compositions for administration is dependent on the mode of administration and can readily be determined by one of ordinary skill in the art. In various aspects, the pharmaceutical composition is sterile or sterilizable. The therapeutic compositions featured in the invention can contain carriers or excipients, many of which are known to skilled artisans. Excipients that can be used include buffers (for example, citrate buffer, phosphate buffer, acetate buffer, and bicarbonate buffer), amino acids, urea, alcohols, ascorbic acid, phospholipids, polypeptides (for example, serum albumin), EDTA, sodium chloride, liposomes, mannitol, sorbitol, water, and glycerol. The nucleic acids, polypeptides, small molecules, and other modulatory compounds featured in the invention can be administered by any standard route of administration. For example, administration can be parenteral, intravenous, subcutaneous, or oral. A modulatory compound can be formulated in various ways, according to the corresponding route of administration. For example, liquid solutions can be made for administration by drops into the ear, for injection, or for ingestion; gels or powders can be made for ingestion or topical application. Methods for making such formulations are well known and can be found in, for example, Remington's Pharmaceutical Sciences, 18th Ed., Gennaro, ed., Mack Publishing Co., Easton, Pa. 1990.

In various aspects, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

In various aspects, the pharmaceutical compositions of this invention can include a pharmaceutically acceptable carrier and a compound or a pharmaceutically acceptable salt of the compounds of the invention. The compounds of the invention, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present invention comprise a compound of the invention (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

In various aspects, the agent that modulates EGFR signaling is an EGFR inhibitor. In a further aspect, the EGFR inhibitor is a tyrosine kinase inhibitor. Examples of tyrosine kinase inhibitors include, but are not limited to, erlotinib. In a still further aspect, the EGFR inhibitor is a monoclonal antibody.

In various aspects, the EGFR inhibitor is selected from erlotinib, afatinib, cetuximab, panitumumab, erlotinib HCl, gefitinib, lapatinib, neratinib, lifirafenib, HER2-inhibitor-1, nazartinib, naquotinib, canertinib, AG-490, CP-724714, Dacomitinib, WZ4002, Sapitinib, CUDC-101, AG-1478, PD153035 HCl, pelitinib, AC480, AEE788, AP261 13-analog, OSI-420, WZ3146, WZ8040, AST-1306, rociletinib, genisten, varlitinib, icotinib, TAK-285, WHI-P154, daphnetin, PD168393, tyrphostin9, CNX-2006, AG-18, AZ5104, osimertinib, CL-387785, olmutinib, AZD3759, poziotinib, vandetanib, and necitumumab.

In a further aspect, the agent that modulates Nrf2 signaling is a Nrf2 inhibitor. In a still further aspect, wherein the Nrf2 inhibitor is selected from isoniazid, ML385, and ethionamide. In yet a further aspect, the Nrf2 inhibitor is isoniazid.

In a further aspect, the agent that modulates Nrf2 signaling also modulates xCT/SLC7A11 signaling. In a still further aspect, the agent that modulates Nrf2 signaling also inhibits xCT/SLCA11 signaling. In yet a further aspect, the agent that modulates Nrf2 signaling and inhibits xCT/SLCA11 signaling is selected from sulfasalazine and erastin.

In a further aspect, the agent that modulates EGFR signaling is an EGFR inhibitor and wherein the agent that modulates Nrf2 signaling is an Nrf2 inhibitor. In a still further aspect, the agent that modulates EGFR signaling is erlotinib and wherein the agent that modulates Nrf2 signaling is isoniazid.

In a further aspect, the agent that modulates EGFR signaling and the agent that modulates Nrf2 signaling are co-formulated. In a still further aspect, the agent that modulates EGFR signaling and the agent that modulates Nrf2 signaling are co-packaged.

In a further aspect, the agent that modulates EGFR signaling and the agent that modulates Nrf2 signaling are administered concurrently. In a still further aspect, the agent that modulates EGFR signaling and the agent that modulates Nrf2 signaling are not administered concurrently.

In a further aspect, an effective amount is a therapeutically effective amount. In a still further aspect, an effective amount is a prophylactically effective amount.

In a further aspect, the effective amount is an individually effective amount of the agent that modulates EGFR signaling or the agent that modulates Nrf2 signaling. In a still further aspect, the effective amount is a combinatorically effective amount of the agent that modulates EGFR signaling and the agent that modulates Nrf2 signaling.

In a further aspect, the pharmaceutical composition is administered to a mammal. In a still further aspect, the mammal is a human. In an even further aspect, the human is a patient.

In a further aspect, the pharmaceutical composition is used to treat gliomas such as, for example, a malignant glioma.

In a further aspect, the glioma expresses EGFR wild type. In a still further aspect, the glioma expresses EGFR mutant. In yet a further aspect, the glioma is resistant to EGFR inhibition.

In various aspects, the composition is a solid dosage form. In a further aspect, the composition is an oral solid dosage form. In a still further aspect, the solid dosage form is a tablet. In yet a further aspect, the solid dosage form is a capsule.

In various aspects, the composition is an injectable dosage form.

It is understood that the disclosed compositions can be prepared from the disclosed compounds. It is also understood that the disclosed compositions can be employed in the disclosed methods of using.

### C. METHODS FOR MAKING A PHARMACEUTICAL COMPOSITION

In one aspect, disclosed are methods for making a pharmaceutical composition, the method comprising combining: (a) an agent that modulates EGFR signaling, or a pharmaceutically acceptable salt thereof; (b) an agent that modulates Nrf2 signaling, or a pharmaceutically acceptable salt thereof; and (c) a pharmaceutically acceptable carrier, wherein at least one of the agent that modulates EGFR signaling and the agent that modulates Nrf2 signaling is present in an effective amount.

In various aspects, the agent that modulates EGFR signaling is an EGFR inhibitor. In a further aspect, the EGFR inhibitor is a tyrosine kinase inhibitor. Examples of tyrosine kinase inhibitors include, but are not limited to, erlotinib. In a still further aspect, the EGFR inhibitor is a monoclonal antibody.

In various aspects, the EGFR inhibitor is selected from erlotinib, afatinib, cetuximab, panitumumab, erlotinib HCl, gefitinib, lapatinib, neratinib, lifirafenib, HER2-inhibitor-1, nazartinib, naquotinib, canertinib, AG-490, CP-724714, Dacomitinib, WZ4002, Sapitinib, CUDC-101, AG-1478, PD153035 HCl, pelitinib, AC480, AEE788, AP261 13-analog, OSI-420, WZ3146, WZ8040, AST-1306, rociletinib, genisten, varlitinib, icotinib, TAK-285, WHI-P154, daphnetin, PD168393, tyrphostin9, CNX-2006, AG-18, AZ5104, osimertinib, CL-387785, olmutinib, AZD3759, poziotinib, vandetanib, and necitumumab.

In a further aspect, the agent that modulates Nrf2 signaling is a Nrf2 inhibitor. In a still further aspect, wherein the Nrf2 inhibitor is selected from isoniazid, ML385, and ethionamide. In yet a further aspect, the Nrf2 inhibitor is isoniazid.

In a further aspect, the agent that modulates Nrf2 signaling also modulates xCT/SLC7A11 signaling. In a still further aspect, the agent that modulates Nrf2 signaling also inhibits xCT/SLCA11 signaling. In yet a further aspect, the agent that modulates Nrf2 signaling and inhibits xCT/SLCA11 signaling is selected from sulfasalazine and erastin.

In a further aspect, the agent that modulates EGFR signaling is an EGFR inhibitor and wherein the agent that modulates Nrf2 signaling is an Nrf2 inhibitor. In a still further aspect, the agent that modulates EGFR signaling is erlotinib and wherein the agent that modulates Nrf2 signaling is isoniazid.

In a further aspect, the agent that modulates EGFR signaling and the agent that modulates Nrf2 signaling are co-formulated. In a still further aspect, the agent that modulates EGFR signaling and the agent that modulates Nrf2 signaling are co-packaged.

In a further aspect, the agent that modulates EGFR signaling and the agent that modulates Nrf2 signaling are administered concurrently. In a still further aspect, the agent that modulates EGFR signaling and the agent that modulates Nrf2 signaling are not administered concurrently.

In a further aspect, an effective amount is a therapeutically effective amount. In a still further aspect, an effective amount is a prophylactically effective amount.

In various aspects, the effective amount is an individually effective amount of the agent that modulates EGFR signaling or the agent that modulates Nrf2 signaling. In a further aspect, the effective amount is an individually effective amount of the agent that modulates EGFR signaling. In a still further aspect, the effective amount is an individually effective amount of the agent that modulates Nrf2 signaling.

In various aspects, the effective amount is a combinatorically effective amount of the agent that modulates EGFR signaling and the agent that modulates Nrf2 signaling.

In various aspects, combining is co-formulating the agent that modulates EGFR signaling and the agent that modulates Nrf2 signaling with the pharmaceutically acceptable carrier. In a further aspect, co-formulating provides an oral solid dosage form comprising the agent that modulates EGFR signaling, the agent that modulates Nrf2 signaling, and the pharmaceutically acceptable carrier. In a still further aspect, the solid dosage form is a tablet. In yet a further aspect, the solid dosage form is a capsule.

In various aspects, co-formulating provides an injectable dosage form comprising the agent that modulates EGFR signaling, the agent that modulates Nrf2 signaling, and the pharmaceutically acceptable carrier.

### D. METHODS FOR TREATING GLIOMA

In one aspect, disclosed are methods for treating glioma in a subject, the method comprising administering to the subject an effective amount of an agent that modulates epidermal growth factor receptor (EGFR) signaling, or a pharmaceutically acceptable salt thereof, and an agent that modulates Nrf2 signaling, or a pharmaceutically acceptable salt thereof.

In one aspect, disclosed are methods for treating a malignant glioma in a patient in need thereof, said method comprising administering to said patient an effective amount of erlotinib and isoniazid.

In various aspects, the agent that modulates EGFR signaling is an EGFR inhibitor. In a further aspect, the EGFR inhibitor is a tyrosine kinase inhibitor. Examples of tyrosine kinase inhibitors include, but are not limited to, erlotinib. In a still further aspect, the EGFR inhibitor is a monoclonal antibody.

In various aspects, the EGFR inhibitor is selected from erlotinib, afatinib, cetuximab, panitumumab, erlotinib HCl, gefitinib, lapatinib, neratinib, lifirafenib, HER2-inhibitor-1, nazartinib, naquotinib, canertinib, AG-490, CP-724714, Dacomitinib, WZ4002, Sapitinib, CUDC-101, AG-1478, PD153035 HCl, pelitinib, AC480, AEE788, AP261 13-analog, OSI-420, WZ3146, WZ8040, AST-1306, rociletinib, genisten, varlitinib, icotinib, TAK-285, WHI-P154, daphnetin, PD168393, tyrphostin9, CNX-2006, AG-18, AZ5104, osimertinib, CL-387785, olmutinib, AZD3759, poziotinib, vandetanib, and necitumumab.

In a further aspect, the agent that modulates Nrf2 signaling is a Nrf2 inhibitor. In a still further aspect, wherein the Nrf2 inhibitor is selected from isoniazid, ML385, and ethionamide. In yet a further aspect, the Nrf2 inhibitor is isoniazid.

In a further aspect, the agent that modulates Nrf2 signaling also modulates xCT/SLC7A11 signaling. In a still further aspect, the agent that modulates Nrf2 signaling also inhibits xCT/SLCA11 signaling. In yet a further aspect, the agent that modulates Nrf2 signaling and inhibits xCT/SLCA11 signaling is selected from sulfasalazine and erastin.

In a further aspect, the agent that modulates EGFR signaling is an EGFR inhibitor and wherein the agent that modulates Nrf2 signaling is an Nrf2 inhibitor. In a still further aspect, the agent that modulates EGFR signaling is erlotinib and wherein the agent that modulates Nrf2 signaling is isoniazid.

In a further aspect, the agent that modulates EGFR signaling and the agent that modulates Nrf2 signaling are co-formulated. In a still further aspect, the agent that modulates EGFR signaling and the agent that modulates Nrf2 signaling are co-packaged.

In a further aspect, the agent that modulates EGFR signaling and the agent that modulates Nrf2 signaling are administered concurrently. In a still further aspect, the agent that modulates EGFR signaling and the agent that modulates Nrf2 signaling are not administered concurrently.

In various aspects, the effective amount is a therapeutically effective amount. In a further aspect, the effective amount is a prophylactically effective amount.

In various aspects, the effective amount is an individually effective amount of the agent that modulates EGFR signaling or the agent that modulates Nrf2 signaling. In a further aspect, the effective amount is an individually effective amount of the agent that modulates EGFR signaling. In a still further aspect, the effective amount is an individually effective amount of the agent that modulates Nrf2 signaling.

In various aspects, the effective amount is a combinatorically effective amount of the agent that modulates EGFR signaling and the agent that modulates Nrf2 signaling.

In a further aspect, the subject has been diagnosed with a need for treatment of glioma prior to the administering step. In a still further aspect, the subject is at risk for developing glioma prior to the administering step.

In a further aspect, the subject is a mammal. In a still further aspect, the mammal is a human.

In a further aspect, the method further comprises the step of identifying a subject in need of treatment of glioma.

In a further aspect, the glioma expresses EGFR wild type. In a still further aspect, the glioma expresses EGFR mutant. In yet a further aspect, the glioma is resistant to EGFR inhibition.

In a further aspect, the method further comprises the step of administering a therapeutically effective amount of at least one chemotherapeutic agent. In yet a further aspect, the chemotherapeutic agent is selected from an alkylating agent, an antimetabolite agent, an antineoplastic antibiotic agent, a mitotic inhibitor agent, and an mTor inhibitor agent.

In various aspects, the antineoplastic antibiotic agent is selected from doxorubicin, mitoxantrone, bleomycin, daunorubicin, dactinomycin, epirubicin, idarubicin, plicamycin, mitomycin, pentostatin, and valrubicin, or a pharmaceutically acceptable salt thereof.

In various aspects, the antimetabolite agent is selected from gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, and thioguanine, or a pharmaceutically acceptable salt thereof.

In various aspects, the alkylating agent is selected from carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, and streptozocin, or a pharmaceutically acceptable salt thereof.

In various aspects, the mitotic inhibitor agent is selected from irinotecan, topotecan, rubitecan, cabazitaxel, docetaxel, paclitaxel, etopside, vincristine, ixabepilone, vinorelbine, vinblastine, and teniposide, or a pharmaceutically acceptable salt thereof.

In various aspects, the mTor inhibitor agent is selected from everolimus, siroliumus, and temsirolimus, or a pharmaceutically acceptable salt, hydrate, solvate, or polymorph thereof.

### E. ADDITIONAL METHODS OF USING THE COMPOUNDS

The compounds and pharmaceutical compositions of the invention are useful in treating or controlling gliomas such as, for example, malignant gliomas.

To treat or control glioma, the compounds and pharmaceutical compositions comprising the compounds are administered to a subject in need thereof, such as a vertebrate, e.g., a mammal, a fish, a bird, a reptile, or an amphibian. The subject can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. The subject is preferably a mammal, such as a human. Prior to administering the compounds or compositions, the subject can be diagnosed with a need for treatment of glioma.

The compounds or compositions can be administered to the subject according to any method. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration and parenteral administration, including injectable such as intravenous administration, intra-arterial administration, intramuscular administration, and subcutaneous administration. Administration can be continuous or intermittent. A preparation can be administered therapeutically; that is, administered to treat an existing disease or condition. A preparation can also be administered prophylactically; that is, administered for prevention of glioma.

The therapeutically effective amount or dosage of the compound can vary within wide limits. Such a dosage is adjusted to the individual requirements in each particular case including the specific compound(s) being administered, the route of administration, the condition being treated, as well as the patient being treated. In general, in the case of oral or parenteral administration to adult humans weighing approximately 70 Kg or more, a daily dosage of about 10 mg to about 10,000 mg, preferably from about 200 mg to about 1,000 mg, should be appropriate, although the upper limit may be exceeded. The daily dosage can be administered as a single dose or in divided doses, or for parenteral administration, as a continuous infusion. Single dose compositions can contain such amounts or submultiples thereof of the compound or composition to make up the daily dose. The dosage can be adjusted by the individual physician in the event of any contraindications. Dosage can vary, and can be administered in one or more dose administrations daily, for one or several days.

1. Use of Agents and Compositions

In one aspect, the invention relates to the use of a disclosed agent, a disclosed pharmaceutical composition, or a product of a disclosed method. In a further aspect, a use relates to the manufacture of a medicament for the treatment of glioma in a subject.

Also provided are the uses of the disclosed agents, compositions, and products. In one aspect, the invention relates to use of at least one disclosed agent, or a pharmaceutically acceptable salt, hydrate, solvate, or polymorph thereof, or at least one disclosed composition. In a further aspect, the composition used is a product of a disclosed method of making.

In a further aspect, the use relates to a process for preparing a pharmaceutical composition comprising a therapeutically effective amount of a disclosed agent or a product of a disclosed method of making, or a pharmaceutically acceptable salt, solvate, or polymorph thereof, for use as a medicament.

In a further aspect, the use relates to a process for preparing a pharmaceutical composition comprising a therapeutically effective amount of a disclosed agent or a product of a disclosed method of making, or a pharmaceutically acceptable salt, solvate, or polymorph thereof, wherein a pharmaceutically acceptable carrier is intimately mixed with a therapeutically effective amount of the compound or the product of a disclosed method of making.

In various aspects, the use relates to a treatment of glioma in a subject. In one aspect, the use is characterized in that the subject is a human. In one aspect, the use is characterized in that the glioma is a malignant glioma.

In a further aspect, the use relates to the manufacture of a medicament for the treatment of glioma in a subject.

It is understood that the disclosed uses can be employed in connection with the disclosed agents, products of disclosed methods of making, methods, compositions, and kits. In a further aspect, the invention relates to the use of a disclosed agents or a disclosed product in the manufacture of a medicament for the treatment of glioma in a mammal. In a further aspect, the glioma is a malignant glioma.

2. Manufacture of a Medicament

In one aspect, the invention relates to a method for the manufacture of a medicament for treating glioma in a subject in need thereof, the method comprising combining a therapeutically effective amount of a disclosed agent, composition, or product of a disclosed method with a pharmaceutically acceptable carrier or diluent.

As regards these applications, the present method includes the administration to an animal, particularly a mammal, and more particularly a human, of a therapeutically effective amount of the agents effective in the treatment of glioma. The dose administered to an animal, particularly a human, in the context of the present invention should be sufficient to affect a therapeutic response in the animal over a reasonable timeframe. One skilled in the art will recognize that dosage will depend upon a variety of factors including the condition of the animal and the body weight of the animal.

The total amount of the agent of the present disclosure administered in a typical treatment is preferably between about 0.05 mg/kg and about 100 mg/kg of body weight for mice, and more preferably between 0.05 mg/kg and about 50 mg/kg of body weight for mice, and between about 100 mg/kg and about 500 mg/kg of body weight, and more preferably between 200 mg/kg and about 400 mg/kg of body weight for humans per daily dose. This total amount is typically, but not necessarily, administered as a series of smaller doses over a period of about one time per day to about three times per day for about 24 months, and preferably over a period of twice per day for about 12 months.

The size of the dose also will be determined by the route, timing and frequency of administration as well as the existence, nature and extent of any adverse side effects that might accompany the administration of the agent or composition and the desired physiological effect. It will be appreciated by one of skill in the art that various conditions or disease states, in particular chronic conditions or disease states, may require prolonged treatment involving multiple administrations.

Thus, in one aspect, the invention relates to the manufacture of a medicament comprising combining a disclosed agent, composition, or a product of a disclosed method of making, or a pharmaceutically acceptable salt, solvate, or polymorph thereof, with a pharmaceutically acceptable carrier or diluent.

3. Kits

In one aspect, disclosed are kits comprising an agent that modulates EGFR signaling, or a pharmaceutically acceptable salt thereof, and an agent that modulates Nrf2 signaling, or a pharmaceutically acceptable salt thereof, and one or more of: (a) an agent associated with the treatment of cancer; (b) instructions for administering the agent that modulates EGFR signaling and/or the agent that modulates Nrf2 signaling in connection with treating glioma; and (c) instructions for treating cancer.

In various aspects, the agent that modulates EGFR signaling is an EGFR inhibitor. In a further aspect, the EGFR inhibitor is a tyrosine kinase inhibitor. Examples of tyrosine kinase inhibitors include, but are not limited to, erlotinib. In a still further aspect, the EGFR inhibitor is a monoclonal antibody.

In various aspects, the EGFR inhibitor is selected from erlotinib, afatinib, cetuximab, panitumumab, erlotinib HCl, gefitinib, lapatinib, neratinib, lifirafenib, HER2-inhibitor-1, nazartinib, naquotinib, canertinib, AG-490, CP-724714, Dacomitinib, WZ4002, Sapitinib, CUDC-101, AG-1478, PD153035 HCl, pelitinib, AC480, AEE788, AP261 13-analog, OSI-420, WZ3146, WZ8040, AST-1306, rociletinib, genisten, varlitinib, icotinib, TAK-285, WHI-P154, daphnetin, PD168393, tyrphostin9, CNX-2006, AG-18, AZ5104, osimertinib, CL-387785, olmutinib, AZD3759, poziotinib, vandetanib, and necitumumab.

In a further aspect, the agent that modulates Nrf2 signaling is a Nrf2 inhibitor. In a still further aspect, wherein the Nrf2 inhibitor is selected from isoniazid, ML385, and ethionamide. In yet a further aspect, the Nrf2 inhibitor is isoniazid.

In a further aspect, the agent that modulates Nrf2 signaling also modulates xCT/SLC7A11 signaling. In a still further aspect, the agent that modulates Nrf2 signaling also inhibits xCT/SLCA11 signaling. In yet a further aspect, the agent that modulates Nrf2 signaling and inhibits xCT/SLCA11 signaling is selected from sulfasalazine and erastin.

In a further aspect, the agent that modulates EGFR signaling is an EGFR inhibitor and wherein the agent that modulates Nrf2 signaling is an Nrf2 inhibitor. In a still further aspect, the agent that modulates EGFR signaling is erlotinib and wherein the agent that modulates Nrf2 signaling is isoniazid.

In a further aspect, the agent that modulates EGFR signaling and the agent that modulates Nrf2 signaling are co-formulated. In a still further aspect, the agent that modulates EGFR signaling and the agent that modulates Nrf2 signaling are co-packaged.

In a further aspect, the agent that modulates EGFR signaling and the agent that modulates Nrf2 signaling are administered concurrently. In a still further aspect, the agent that modulates EGFR signaling and the agent that modulates Nrf2 signaling are not administered concurrently.

In a further aspect, the glioma is a malignant glioma.

In a further aspect, the agent is a chemotherapeutic agent. In yet a further aspect, the chemotherapeutic agent is selected from an alkylating agent, an antimetabolite agent, an antineoplastic antibiotic agent, a mitotic inhibitor agent, and a mTor inhibitor agent.

In various aspects, the antineoplastic antibiotic agent is selected from doxorubicin, mitoxantrone, bleomycin, daunorubicin, dactinomycin, epirubicin, idarubicin, plicamycin, mitomycin, pentostatin, and valrubicin, or a pharmaceutically acceptable salt thereof.

In various aspects, the antimetabolite agent is selected from gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, and thioguanine, or a pharmaceutically acceptable salt thereof.

In various aspects, the alkylating agent is selected from carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, and streptozocin, or a pharmaceutically acceptable salt thereof.

In various aspects, the mitotic inhibitor agent is selected from irinotecan, topotecan, rubitecan, cabazitaxel, docetaxel, paclitaxel, etopside, vincristine, ixabepilone, vinorelbine, vinblastine, and teniposide, or a pharmaceutically acceptable salt thereof.

In various aspects, the mTor inhibitor agent is selected from everolimus, siroliumus, and temsirolimus, or a pharmaceutically acceptable salt, hydrate, solvate, or polymorph thereof.

In various aspects, the agent that modulates EGFR signaling and the agent that modulates Nrf2 signaling are administered sequentially. In a further aspect, the agent that modulates EGFR signaling and the agent that modulates Nrf2 signaling are administered simultaneously.

In various aspects, the agent that modulates EGFR signaling and the chemotherapeutic agent are administered sequentially. In a further aspect, the agent that modulates EGFR signaling and the chemotherapeutic agent are administered simultaneously.

In various aspects, the agent that modulates Nrf2 signaling and the chemotherapeutic agent are administered sequentially. In a further aspect, the agent that modulates Nrf2 signaling and the chemotherapeutic agent are administered simultaneously.

In various aspects, the agent that modulates EGFR signaling, the agent that modulates Nrf2 signaling, and the chemotherapeutic agent are administered sequentially. In a further aspect, the agent that modulates EGFR signaling, the agent that modulates Nrf2 signaling, and the chemotherapeutic agent are administered simultaneously.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is understood that the disclosed kits can be prepared from the disclosed compounds, products, and pharmaceutical compositions. It is also understood that the disclosed kits can be employed in connection with the disclosed methods of using.

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

### F. REFERENCES

An, Z., Aksoy, O., Zheng, T., Fan, Q. W., and Weiss, W. A. (2018) Epidermal growth factor receptor and EGFRvIII in glioblastoma: signaling pathways and targeted therapies, Oncogene 37, 1561-1575.
Hatanpaa, K. J., Burma S., Zhao D., Habib, A. A. (2010) Epidermal growth factor receptor (EGFR) in glioma: Signal transduction, neuropathology, imaging and radioresistance Neoplasia 12, 675-684.
Huang, P. H., Xu, A. M., and White, F. M. (2009) Oncogenic EGFR signaling networks in glioma, Sci Signal 2, re6.
Acquaviva, J., Jun, H. J., Lessard, J., Ruiz, R., Zhu, H., Donovan, M., Woolfenden, S., Boskovitz, A., Raval, A., Bronson, R. T., Pfannl, R., Whittaker, C. A., Housman, D. E., and Charest, A. (2011) Chronic activation of wild-type epidermal growth factor receptor and loss of Cdkn2a cause mouse glioblastoma formation, Cancer Res 71, 7198-7206.
Furnari, F. B., Fenton, T., Bachoo, R. M., Mukasa, A., Stommel, J. M., Stegh, A., Hahn, W. C., Ligon, K. L., Louis, D. N., Brennan, C., Chin, L., DePinho, R. A., and Cavenee, W. K. (2007) Malignant astrocytic glioma: genetics, biology, and paths to treatment, Genes Dev 21, 2683-2710.
Li, L., Chakraborty, S., Yang, C. R., Hatanpaa, K. J., Cipher, D. J., Puliyappadamba, V. T., Rehman, A., Jiwani, A. J., Mickey, B., Madden, C., Raisanen, J., Burma, S., Saha, D., Wang, Z., Pingle, S. C., Kesari, S., Boothman, D. A., and Habib, A. A. (2014) An EGFR wild type-EGFRvIII-HB-EGF feed-forward loop regulates the activation of EGFRvIII, Oncogene 33, 4253-4264.
Fan, Q. W., Cheng, C. K., Gustafson, W. C., Charron, E., Zipper, P., Wong, R. A., Chen, J., Lau, J., Knobbe-Thomsen, C., Weller, M., Jura, N., Reifenberger, G., Shokat, K. M., and Weiss, W. A. (2013) EGFR Phosphorylates Tumor-Derived EGFRvIII Driving STAT3/5 and Progression in Glioblastoma, Cancer Cell 24, 438-449.
Weller, M., Butowski, N., Tran, D. D., Recht, L. D., Lim, M., Hirte, H., Ashby, L., Mechtler, L., Goldlust, S. A., Iwamoto, F., Drappatz, J., O'Rourke, D. M., Wong, M., Hamilton, M. G., Finocchiaro, G., Perry, J., Wick, W., Green, J., He, Y., Turner, C. D., Yellin, M. J., Keler, T., Davis, T. A., Stupp, R., Sampson, J. H., and investigators, A. I. t. (2017) Rindopepimut with temozolomide for patients with newly diagnosed, EGFRvIII-expressing glioblastoma (ACT IV): a randomised, double-blind, international phase 3 trial, The lancet oncology 18, 1373-1385.
Karpel-Massler, G., Schmidt, U., Unterberg, A., and Halatsch, M. E. (2009) Therapeutic inhibition of the epidermal growth factor receptor in high-grade gliomas: where do we stand?, Mol Cancer Res 7, 1000-1012.
Roth, P., and Weller, M. (2014) Challenges to targeting epidermal growth factor receptor in glioblastoma: escape mechanisms and combinatorial treatment strategies, Neuro Oncol 16 Suppl 8, viii 14-19.
Yu, H. A., Arcila, M. E., Rekhtman, N., Sima, C. S., Zakowski, M. F., Pao, W., Kris, M. G., Miller, V. A., Ladanyi, M., and Riely, G. J. (2013) Analysis of tumor specimens at the time of acquired resistance to EGFR-TKI therapy in 155 patients with EGFR-mutant lung cancers, Clin Cancer Res 19, 2240-2247.
Engelman, J. A., Zejnullahu, K., Mitsudomi, T., Song, Y., Hyland, C., Park, J. O., Lindeman, N., Gale, C. M., Zhao, X., Christensen, J., Kosaka, T., Holmes, A. J., Rogers, A. M., Cappuzzo, F., Mok, T., Lee, C., Johnson, B. E., Cantley, L. C., and Janne, P. A. (2007) MET amplification leads to gefitinib resistance in lung cancer by activating ERBB3 signaling, Science 316, 1039-1043.
Le, X., Puri, S., Negrao, M. V., Nilsson, M. B., Robichaux, J., Boyle, T., Hicks, J. K., Lovinger, K. L., Roarty, E., Rinsurongkawong, W., Tang, M., Sun, H., Elamin, Y., Lacerda, L. C., Lewis, J., Roth, J. A., Swisher, S. G., Lee, J. J., William, W. N., Jr., Glisson, B. S., Zhang, J., Papadimitrakopoulou, V. A., Gray, J. E., and Heymach, J. V. (2018) Landscape of EGFR-Dependent and -Independent Resistance Mechanisms to Osimertinib and Continuation Therapy Beyond Progression in EGFR-Mutant NSCLC, Clin Cancer Res 24, 6195-6203.
Sun, C., and Bernards, R. (2014) Feedback and redundancy in receptor tyrosine kinase signaling: relevance to cancer therapies, Trends in biochemical sciences 39,
Frederick, L., Wang, X. Y., Eley, G., and James, C. D. (2000) Diversity and frequency of epidermal growth factor receptor mutations in human glioblastomas, Cancer Res 60, 1383-1387.
Biernat, W., Huang, H., Yokoo, H., Kleihues, P., and Ohgaki, H. (2004) Predominant expression of mutant EGFR (EGFRvIII) is rare in primary glioblastomas, Brain pathology 14, 131-136.
Ramnarain, D. B., Park, S., Lee, D. Y., Hatanpaa, K. J., Scoggin, S. O., Otu, H., Libermann, T. A., Raisanen, J. M., Ashfaq, R., Wong, E. T., Wu, J., Elliott, R., and Habib, A. A. (2006) Differential gene expression analysis reveals generation of an autocrine loop by a mutant epidermal growth factor receptor in glioma cells, Cancer Res 66, 867-874.
Inda, M. D., Bonavia, R., Mukasa, A., Narita, Y., Sah, D. W., Vandenberg, S., Brennan, C., Johns, T. G., Bachoo, R., Hadwiger, P., Tan, P., Depinho, R. A., Cavenee, W., and Furnari, F. (2010) Tumor heterogeneity is an active process maintained by a mutant EGFR-induced cytokine circuit in glioblastoma, Genes Dev 24, 1731-1745.
Guo, G., Gong, K., Ali, S., Ali, N., Shallwani, S., Hatanpaa, K. J., Pan, E., Mickey, B., Burma, S., Wang, D. H., Kesari, S., Sarkaria, J. N., Zhao, D., and Habib, A. A. (2017) A TNF-JNK-Axl-ERK signaling axis mediates primary resistance to EGFR inhibition in glioblastoma, Nat Neurosci 20, 1074-1084.
Warta, R., and Herold-Mende, C. (2017) Helping EGFR inhibition to block cancer, Nat Neurosci 20, 1035-1037.
Mai, W. X., Gosa, L., Daniels, V. W., Ta, L., Tsang, J. E., Higgins, B., Gilmore, W. B., Bayley, N. A., Harati, M. D., Lee, J. T., Yong, W. H., Kornblum, H. I., Bensinger, S. J., Mischel, P. S., Rao, P. N., Clark, P. M., Cloughesy, T. F., Letai, A., and Nathanson, D. A. (2017) Cytoplasmic p53 couples oncogene-driven glucose metabolism to apoptosis and is a therapeutic target in glioblastoma, Nature medicine 23, 1342-1351.
Guo, G., Gong, K., Puliyapaddamba, V. T., Panchani, N., Pan, E., Mukherjee, B., Damanwalla, Z., Bharia, S., Hatanpaa, K. J., Gerber, D. E., Mickey, B. E., Patel, T. R., Sarkaria, J. N., Zhao, D., Burma, S., and Habib, A. A. (2019) Efficacy of EGFR plus TNF inhibition in a preclinical model of temozolomide-resistant glioblastoma, Neuro Oncol. 2019 Jul. 31. pii: noz127. doi: 10.1093/neuonc/noz127. [Epub ahead of print]
Johannessen, T. A., and Bjerkvig, R. (2019) A new chance for EGFR inhibition in glioblastoma?, Neuro Oncol. 2019 Oct. 19. pii: noz196. doi: 10.1093/neuonc/noz196. [Epub ahead of print]
Nathanson, D. A., Gini, B., Mottahedeh, J., Visnyei, K., Koga, T., Gomez, G., Eskin, A., Hwang, K., Wang, J., Masui, K., Paucar, A., Yang, H., Ohashi, M., Zhu, S., Wykosky, J., Reed, R., Nelson, S. F., Cloughesy, T. F., James, C. D., Rao, P. N., Kornblum, H. I., Heath, J. R., Cavenee, W. K., Furnari, F. B., and Mischel, P. S. (2014) Targeted therapy resistance mediated by dynamic regulation of extrachromosomal mutant EGFR DNA, Science 343, 72-76.
Wykosky, J., Hu, J., Gomez, G. G., Taylor, T., Villa, G. R., Pizzo, D., VandenBerg, S. R., Thorne, A. H., Chen, C. C., Mischel, P. S., Gonias, S. L., Cavenee, W. K., and Furnari, F. B. (2015) A urokinase receptor-Bim signaling axis emerges during EGFR inhibitor resistance in mutant EGFR glioblastoma, Cancer Res 75, 394-404.
Zanca, C., Villa, G. R., Benitez, J. A., Thorne, A. H., Koga, T., D'Antonio, M., Ikegami, S., Ma, J., Boyer, A. D., Banisadr, A., Jameson, N. M., Parisian, A. D., Eliseeva, 0. V., Barnabe, G. F., Liu, F., Wu, S., Yang, H., Wykosky, J., Frazer, K. A., Verkhusha, V. V., Isaguliants, M. G., Weiss, W. A., Gahman, T. C., Shiau, A. K., Chen, C. C., Mischel, P. S., Cavenee, W. K., and Furnari, F. B. (2017) Glioblastoma cellular cross-talk converges on NF-kappaB to attenuate EGFR inhibitor sensitivity, Genes Dev 31, 1212-1227.
Ahmad, F., Dixit, D., Sharma, V., Kumar, A., Joshi, S. D., Sarkar, C., and Sen, E. (2016) Nrf2-driven TERT regulates pentose phosphate pathway in glioblastoma, Cell Death Dis 7, e2213.
Shi, L., Li, H., and Than, Y. (2017) All-trans retinoic acid enhances temozolomide-induced autophagy in human glioma cells U251 via targeting Keap1/Nrf2/ARE signaling pathway, Oncol Lett 14, 2709-2714.
Zhang, L., and Wang, H. (2017) FTY720 inhibits the Nrf2/ARE pathway in human glioblastoma cell lines and sensitizes glioblastoma cells to temozolomide, Pharmacol Rep 69, 1186-1193.
Polonen, P., Jawahar Deen, A., Leinonen, H. M., Jyrkkanen, H. K., Kuosmanen, S., Mononen, M., Jain, A., Tuomainen, T., Pasonen-Seppanen, S., Hartikainen, J. M., Mannermaa, A., Nykter, M., Tavi, P., Johansen, T., Heinaniemi, M., and Levonen, A. L. (2019) Nrf2 and SQSTM1/p62 jointly contribute to mesenchymal transition and invasion in glioblastoma, Oncogene.
Zhu, J., Wang, H., Sun, Q., Ji, X., Zhu, L., Cong, Z., Zhou, Y., Liu, H., and Zhou, M. (2013) Nrf2 is required to maintain the self-renewal of glioma stem cells, BMC Cancer 13, 380.
Cong, Z. X., Wang, H. D., Wang, J. W., Zhou, Y., Pan, H., Zhang, D. D., and Zhu, L. (2013) ERK and PI3K signaling cascades induce Nrf2 activation and regulate cell viability partly through Nrf2 in human glioblastoma cells, Oncology reports 30, 715-722.
Kanamori, M., Higa, T., Sonoda, Y., Murakami, S., Dodo, M., Kitamura, H., Taguchi, K., Shibata, T., Watanabe, M., Suzuki, H., Shibahara, I., Saito, R., Yamashita, Y., Kumabe, T., Yamamoto, M., Motohashi, H., and Tominaga, T. (2015) Activation of the NRF2 pathway and its impact on the prognosis of anaplastic glioma patients, Neuro Oncol 17, 555-565.
Rojo de la Vega, M., Chapman, E., and Zhang, D. D. (2018) NRF2 and the Hallmarks of Cancer, Cancer Cell.
Galan-Cobo, A., Sitthideatphaiboon, P., Qu, X., Poteete, A., Pisegna, M. A., Tong, P., Chen, P. H., Boroughs, L. K., Rodriguez, M. L. M., Zhang, W., Parlati, F., Wang, J., Gandhi, V., Skoulidis, F., DeBerardinis, R. J., Minna, J. D., and Heymach, J. V. (2019) LKB1 and KEAP1/NRF2 Pathways Cooperatively Promote Metabolic Reprogramming with Enhanced Glutamine Dependence in KRAS-Mutant Lung Adenocarcinoma, Cancer Res 79, 3251-3267.
Krall, E. B., Wang, B., Munoz, D. M., Ilic, N., Raghavan, S., Niederst, M. J., Yu, K., Ruddy, D. A., Aguirre, A. J., Kim, J. W., Redig, A. J., Gainor, J. F., Williams, J. A., Asara, J. M., Doench, J. G., Janne, P. A., Shaw, A. T., McDonald Iii, R. E., Engelman, J. A., Stegmeier, F., Schlabach, M. R., and Hahn, W. C. (2017) KEAP1 loss modulates sensitivity to kinase targeted therapy in lung cancer, Elife 6.
Park, S. H., Kim, J. H., Ko, E., Kim, J. Y., Park, M. J., Kim, M. J., Seo, H., Li, S., and Lee, J. Y. (2018) Resistance to gefitinib and cross-resistance to irreversible EGFR-TKIs mediated by disruption of the Keap1-Nrf2 pathway in human lung cancer cells, FASEB J, fj201800011R.
Lim, M., Xia, Y., Bettegowda, C., and Weller, M. (2018) Current state of immunotherapy for glioblastoma, Nat Rev Clin Oncol 15, 422-442.
Jackson, C. M., Choi, J., and Lim, M. (2019) Mechanisms of immunotherapy resistance: lessons from glioblastoma, Nat Immunol 20, 1100-1109.
Chen, Y., Xue, P., Hou, Y., Zhang, H., Zheng, H., Zhou, T., Qu, W., Teng, W., Zhang, Q., Andersen, M. E., and Pi, J. (2013) Isoniazid suppresses antioxidant response element activities and impairs adipogenesis in mouse and human preadipocytes, Toxicology and applied pharmacology 273, 435-441.
Verma, A. K., Yadav, A., Dewangan, J., Singh, S. V., Mishra, M., Singh, P. K., and Rath, S. K. (2015) Isoniazid prevents Nrf2 translocation by inhibiting ERK1 phosphorylation and induces oxidative stress and apoptosis, Redox Biol 6, 80-92.
Peng, H., Wang, H., Xue, P., Hou, Y., Dong, J., Zhou, T., Qu, W., Peng, S., Li, J., Carmichael, P. L., Nelson, B., Clewell, R., Zhang, Q., Andersen, M. E., and Pi, J. (2016) Suppression of NRF2-ARE activity sensitizes chemotherapeutic agent-induced cytotoxicity in human acute monocytic leukemia cells, Toxicology and applied pharmacology 292, 1-7.
Nau, R., Sorgel, F., and Eiffert, H. (2010) Penetration of drugs through the blood-cerebrospinal fluid/blood-brain barrier for treatment of central nervous system infections, Clin Microbiol Rev 23, 858-883.
Sullins, A. K., and Abdel-Rahman, S. M. (2013) Pharmacokinetics of antibacterial agents in the CSF of children and adolescents, Paediatr Drugs 15, 93-117.
Hammond, M. E., Hayes, D. F., Wolff, A. C., Mangu, P. B., and Temin, S. (2010) American society of clinical oncology/college of american pathologists guideline recommendations for immunohistochemical testing of estrogen and progesterone receptors in breast cancer, J Oncol Pract 6, 195-197.
Wolff, A. C., Hammond, M. E., Schwartz, J. N., Hagerty, K. L., Allred, D. C., Cote, R. J., Dowsett, M., Fitzgibbons, P. L., Hanna, W. M., Langer, A., McShane, L. M., Paik, S., Pegram, M. D., Perez, E. A., Press, M. F., Rhodes, A., Sturgeon, C., Taube, S. E., Tubbs, R., Vance, G. H., van de Vijver, M., Wheeler, T. M., Hayes, D. F., American Society of Clinical, O., and College of American, P. (2007) American Society of Clinical Oncology/College of American Pathologists guideline recommendations for human epidermal growth factor receptor 2 testing in breast cancer, J Clin Oncol 25, 118-145.
Fruh, M., and Pless, M. (2012) EGFR IHC score for selection of cetuximab treatment: Ready for clinical practice?, Transl Lung Cancer Res 1, 145-146.
Hutchinson, R. A., Adams, R. A., McArt, D. G., Salto-Tellez, M., Jasani, B., and Hamilton, P. W. (2015) Epidermal growth factor receptor immunohistochemistry: new opportunities in metastatic colorectal cancer, J Transl Med 13, 217.
(2008) Comprehensive genomic characterization defines human glioblastoma genes and core pathways, Nature 455, 1061-1068.
Batra, S. K., Castelino-Prabhu, S., Wikstrand, C. J., Zhu, X., Humphrey, P. A., Friedman, H. S., and Bigner, D. D. (1995) Epidermal growth factor ligand-independent, unregulated, cell-transforming potential of a naturally occurring human mutant EGFRvIII gene, Cell Growth Differ 6, 1251-1259.
Di Fiore, P. P., Pierce, J. H., Fleming, T. P., Hazan, R., Ullrich, A., King, C. R., Schlessinger, J., and Aaronson, S. A. (1987) Overexpression of the human EGF receptor confers an EGF-dependent transformed phenotype to NIH 3T3 cells, Cell 51, 1063-1070.
Velu, T. J., Beguinot, L., Vass, W. C., Willingham, M. C., Merlino, G. T., Pastan, I., and Lowy, D. R. (1987) Epidermal-growth-factor-dependent transformation by a human EGF receptor proto-oncogene, Science 238, 1408-1410.
Tang, P., Steck, P. A., and Yung, W. K. (1997) The autocrine loop of TGF-alpha/EGFR and brain tumors, J Neurooncol 35, 303-314.
Ekstrand, A. J., James, C. D., Cavenee, W. K., Seliger, B., Pettersson, R. F., and Collins, V. P. (1991) Genes for epidermal growth factor receptor, transforming growth factor alpha, and epidermal growth factor and their expression in human gliomas in vivo, Cancer Res 51, 2164-2172.
Chakraborty, S., Li, L., Puliyappadamba, V. T., Guo, G., Hatanpaa, K. J., Mickey, B., Souza, R. F., Vo, P., Herz, J., Chen, M. R., Boothman, D. A., Pandita, T. K., Wang, D. H., Sen, G. C., and Habib, A. A. (2014) Constitutive and ligand-induced EGFR signalling triggers distinct and mutually exclusive downstream signalling networks, Nat Commun 5, 5811.
Mishima, K., Higashiyama, S., Asai, A., Yamaoka, K., Nagashima, Y., Taniguchi, N., Kitanaka, C., Kirino, T., and Kuchino, Y. (1998) Heparin-binding epidermal growth factor-like growth factor stimulates mitogenic signaling and is highly expressed in human malignant gliomas, Acta Neuropathol (Berl) 96, 322-328.
Nishikawa, R., Ji, X. D., Harmon, R. C., Lazar, C. S., Gill, G. N., Cavenee, W. K., and Huang, H. J. (1994) A mutant epidermal growth factor receptor common in human glioma confers enhanced tumorigenicity, Proc Natl Acad Sci USA 91, 7727-7731.
Wong, A. J., Ruppert, J. M., Bigner, S. H., Grzeschik, C. H., Humphrey, P. A., Bigner, D. S., and Vogelstein, B. (1992) Structural alterations of the epidermal growth factor receptor gene in human gliomas, Proc Natl Acad Sci USA 89, 2965-2969.
Guo, G., Gong, K., Wohlfeld, B., Hatanpaa, K. J., Zhao, D., and Habib, A. A. (2015) Ligand-Independent EGFR Signaling, Cancer Res 75, 3436-3441.
Endres, N. F., Das, R., Smith, A. W., Arkhipov, A., Kovacs, E., Huang, Y., Pelton, J. G., Shan, Y., Shaw, D. E., Wemmer, D. E., Groves, J. T., and Kuriyan, J. (2013) Conformational coupling across the plasma membrane in activation of the EGF receptor, Cell 152, 543-556.
Nishikawa, R., Sugiyama, T., Narita, Y., Furnari, F., Cavenee, W. K., and Matsutani, M. (2004) Immunohistochemical analysis of the mutant epidermal growth factor, deltaEGFR, in glioblastoma, Brain Tumor Pathol 21, 53-56.
Verhaak, R. G., Hoadley, K. A., Purdom, E., Wang, V., Qi, Y., Wilkerson, M. D., Miller, C. R., Ding, L., Golub, T., Mesirov, J. P., Alexe, G., Lawrence, M., O'Kelly, M., Tamayo, P., Weir, B. A., Gabriel, S., Winckler, W., Gupta, S., Jakkula, L., Feiler, H. S., Hodgson, J. G., James, C. D., Sarkaria, J. N., Brennan, C., Kahn, A., Spellman, P. T., Wilson, R. K., Speed, T. P., Gray, J. W., Meyerson, M., Getz, G., Perou, C. M., and Hayes, D. N. (2010) Integrated genomic analysis identifies clinically relevant subtypes of glioblastoma characterized by abnormalities in PDGFRA, IDH1, EGFR, and NF1, Cancer Cell 17, 98-110.
Rich, J. N., Reardon, D. A., Peery, T., Dowell, J. M., Quinn, J. A., Penne, K. L., Wikstrand, C. J., Van Duyn, L. B., Dancey, J. E., McLendon, R. E., Kao, J. C., Stenzel, T. T., Ahmed Rasheed, B. K., Tourt-Uhlig, S. E., Herndon, J. E., 2nd, Vredenburgh, J. J., Sampson, J. H., Friedman, A. H., Bigner, D. D., and Friedman, H. S. (2004) Phase II trial of gefitinib in recurrent glioblastoma, J Clin Oncol 22, 133-142.
Reardon, D. A., Desjardins, A., Vredenburgh, J. J., Gururangan, S., Friedman, A. H., Herndon, J. E., 2nd, Marcello, J., Norfleet, J. A., McLendon, R. E., Sampson, J. H., and Friedman, H. S. (2010) Phase 2 trial of erlotinib plus sirolimus in adults with recurrent glioblastoma, J Neurooncol 96, 219-230.
Sathornsumetee, S., Desjardins, A., Vredenburgh, J. J., McLendon, R. E., Marcello, J., Herndon, J. E., Mathe, A., Hamilton, M., Rich, J. N., Norfleet, J. A., Gururangan, S., Friedman, H. S., and Reardon, D. A. (2010) Phase II trial of bevacizumab and erlotinib in patients with recurrent malignant glioma, Neuro Oncol 12, 1300-1310.
Brown, P. D., Krishnan, S., Sarkaria, J. N., Wu, W., Jaeckle, K. A., Uhm, J. H., Geoffroy, F. J., Arusell, R., Kitange, G., Jenkins, R. B., Kugler, J. W., Morton, R. F., Rowland, K. M., Jr., Mischel, P., Yong, W. H., Scheithauer, B. W., Schiff, D., Giannini, C., Buckner, J. C., and North Central Cancer Treatment Group Study, N. (2008) Phase I/II trial of erlotinib and temozolomide with radiation therapy in the treatment of newly diagnosed glioblastoma multiforme: North Central Cancer Treatment Group Study N0177, J Clin Oncol 26, 5603-5609.
Peereboom, D. M., Shepard, D. R., Ahluwalia, M. S., Brewer, C. J., Agarwal, N., Stevens, G. H., Suh, J. H., Toms, S. A., Vogelbaum, M. A., Weil, R. J., Elson, P., and Barnett, G. H. (2010) Phase II trial of erlotinib with temozolomide and radiation in patients with newly diagnosed glioblastoma multiforme, J Neurooncol 98, 93-99.
Gong, K., Guo, G., Gerber, D. E., Gao, B., Peyton, M., Huang, C., Minna, J. D., Hatanpaa, K. J., Kernstine, K., Cai, L., Xie, Y., Zhu, H., Fattah, F. J., Zhang, S., Takahashi, M., Mukherjee, B., Burma, S., Dowell, J., Dao, K., Papadimitrakopoulou, V. A., Olivas, V., Bivona, T. G., Zhao, D., and Habib, A. A. (2018) TNF-driven adaptive response mediates resistance to EGFR inhibition in lung cancer, J Clin Invest 128, 2500-2518.
Best, S. A., and Sutherland, K. D. (2018) “Keaping” a lid on lung cancer: the Keap1-Nrf2 pathway, Cell Cycle 17, 1696-1707.
Jeong, Y., Hoang, N. T., Lovejoy, A., Stehr, H., Newman, A. M., Gentles, A. J., Kong, W., Truong, D., Martin, S., Chaudhuri, A., Heiser, D., Zhou, L., Say, C., Carter, J. N., Hiniker, S. M., Loo, B. W., Jr., West, R. B., Beachy, P., Alizadeh, A. A., and Diehn, M. (2017) Role of KEAP1/NRF2 and TP53 Mutations in Lung Squamous Cell Carcinoma Development and Radiation Resistance, Cancer Discov 7, 86-101.
Bai, X., Chen, Y., Hou, X., Huang, M., and Jin, J. (2016) Emerging role of NRF2 in chemoresistance by regulating drug-metabolizing enzymes and efflux transporters, Drug Metab Rev 48, 541-567.
McDonald, J. T., Kim, K., Norris, A. J., Vlashi, E., Phillips, T. M., Lagadec, C., Della Donna, L., Ratikan, J., Szelag, H., Hlatky, L., and McBride, W. H. (2010) Ionizing radiation activates the Nrf2 antioxidant response, Cancer Res 70, 8886-8895.
Polewski, M. D., Reveron-Thornton, R. F., Cherryholmes, G. A., Marinov, G. K., and Aboody, K. S. (2017) SLC7A11 Overexpression in Glioblastoma Is Associated with Increased Cancer Stem Cell-Like Properties, Stem Cells Dev 26, 1236-1246.
Polewski, M. D., Reveron-Thornton, R. F., Cherryholmes, G. A., Marinov, G. K., Cassady, K., and Aboody, K. S. (2016) Increased Expression of System xc- in Glioblastoma Confers an Altered Metabolic State and Temozolomide Resistance, Mol Cancer Res 14, 1229-1242.
Santosh, V., and Sravya, P. (2017) Glioma, glutamate (SLC7A11) and seizures—a commentary, Ann Transl Med 5, 214.
Rocha, C. R., Kajitani, G. S., Quinet, A., Fortunato, R. S., and Menck, C. F. (2016) NRF2 and glutathione are key resistance mediators to temozolomide in glioma and melanoma cells, Oncotarget 7, 48081-48092.
Sorensen, M. F., Heimisdottir, S. B., Sorensen, M. D., Mellegaard, C. S., Wohlleben, H., Kristensen, B. W., and Beier, C. P. (2018) High expression of cystine-glutamate antiporter xCT (SLC7A11) is an independent biomarker for epileptic seizures at diagnosis in glioma, J Neurooncol 138, 49-53.
Robert, S. M., Buckingham, S. C., Campbell, S. L., Robel, S., Holt, K. T., Ogunrinu-Babarinde, T., Warren, P. P., White, D. M., Reid, M. A., Eschbacher, J. M., Berens, M. E., Lahti, A. C., Nabors, L. B., and Sontheimer, H. (2015) SLC7A11 expression is associated with seizures and predicts poor survival in patients with malignant glioma, Sci Transl Med 7, 289ra286.
Silva-Islas, C. A., and Maldonado, P. D. (2018) Canonical and non-canonical mechanisms of Nrf2 activation, Pharmacol Res 134, 92-99.
Chandarlapaty, S., Sawai, A., Scaltriti, M., Rodrik-Outmezguine, V., Grbovic-Huezo, O., Serra, V., Majumder, P. K., Baselga, J., and Rosen, N. (2011) AKT inhibition relieves feedback suppression of receptor tyrosine kinase expression and activity, Cancer Cell 19, 58-71.
Prahallad, A., Sun, C., Huang, S., Di Nicolantonio, F., Salazar, R., Zecchin, D., Beijersbergen, R. L., Bardelli, A., and Bernards, R. (2012) Unresponsiveness of colon cancer to BRAF(V600E) inhibition through feedback activation of EGFR, Nature 483, 100-103.
Duncan, J. S., Whittle, M. C., Nakamura, K., Abell, A. N., Midland, A. A., Zawistowski, J. S., Johnson, N. L., Granger, D. A., Jordan, N. V., Dan, D. B., Usary, J., Kuan, P. F., Smalley, D. M., Major, B., He, X., Hoadley, K. A., Zhou, B., Sharpless, N. E., Perou, C. M., Kim, W. Y., Gomez, S. M., Chen, X., Jin, J., Frye, S. V., Earp, H. S., Graves, L. M., and Johnson, G. L. (2012) Dynamic reprogramming of the kinome in response to targeted MEK inhibition in triple-negative breast cancer, Cell 149, 307-321.
Corcoran, R. B., Ebi, H., Turke, A. B., Coffee, E. M., Nishino, M., Cogdill, A. P., Brown, R. D., Della Pelle, P., Dias-Santagata, D., Hung, K. E., Flaherty, K. T., Pins, A., Wargo, J. A., Settleman, J., Mino-Kenudson, M., and Engelman, J. A. (2012) EGFR-mediated re-activation of MAPK signaling contributes to insensitivity of BRAF mutant colorectal cancers to RAF inhibition with vemurafenib, Cancer Discov 2, 227-235.
Sun, C., Hobor, S., Bertotti, A., Zecchin, D., Huang, S., Galimi, F., Cottino, F., Prahallad, A., Grernrum, W., Tzani, A., Schlicker, A., Wessels, L. F., Smit, E. F., Thunnissen, E., Halonen, P., Lieftink, C., Beijersbergen, R. L., Di Nicolantonio, F., Bardelli, A., Trusolino, L., and Bernards, R. (2014) Intrinsic resistance to MEK inhibition in KRAS mutant lung and colon cancer through transcriptional induction of ERBB3, Cell Rep 7, 86-93.
Akhavan, D., Pourzia, A. L., Nourian, A. A., Williams, K. J., Nathanson, D., Babic, I., Villa, G. R., Tanaka, K., Nael, A., Yang, H., Dang, J., Vinters, H. V., Yong, W. H., Flagg, M., Tamanoi, F., Sasayama, T., James, C. D., Kornblum, H. I., Cloughesy, T. F., Cavenee, W. K., Bensinger, S. J., and Mischel, P. S. (2013) De-repression of PDGFRbeta transcription promotes acquired resistance to EGFR tyrosine kinase inhibitors in glioblastoma patients, Cancer Discov 3, 534-547.
Jahangiri, A., Nguyen, A., Chandra, A., Sidorov, M. K., Yagnik, G., Rick, J., Han, S. W., Chen, W., Flanigan, P. M., Schneidman-Duhovny, D., Mascharak, S., De Lay, M., Imber, B., Park, C. C., Matsumoto, K., Lu, K., Bergers, G., Sali, A., Weiss, W. A., and Aghi, M. K. (2017) Cross-activating c-Met/beta1 integrin complex drives metastasis and invasive resistance in cancer, Proc Natl Acad Sci USA 114, E8685-E8694.
Lu, K. V., and Bergers, G. (2013) Mechanisms of evasive resistance to anti-VEGF therapy in glioblastoma, CNS Oncol 2, 49-65.
Tamura, R., Tanaka, T., Miyake, K., Yoshida, K., and Sasaki, H. (2017) Bevacizumab for malignant gliomas: current indications, mechanisms of action and resistance, and markers of response, Brain Tumor Pathol 34, 62-77.
Hundsberger, T., Reardon, D. A., and Wen, P. Y. (2017) Angiogenesis inhibitors in tackling recurrent glioblastoma, Expert Rev Anticancer Ther 17, 507-515.
Wainwright, D. A., Chang, A. L., Dey, M., Balyasnikova, I. V., Kim, C., Tobias, A. L., Cheng, Y., Kim, J., Zhang, L., Qiao, J., Han, Y., and Lesniak, M. S. (2014) Durable therapeutic efficacy utilizing combinatorial blockade against IDO, CTLA-4 and PD-L1 in mice with brain tumors, Clin Cancer Res.
Majd, N., Kamiya-Matsuoka, C., and de Groot, J. (2019) The path forward for anti-programmed death-1 therapy in gliomas, Curr Opin Neurol.
Russell, L., Swanner, J., Jaime-Ramirez, A. C., Wang, Y., Sprague, A., Banasavadi-Siddegowda, Y., Yoo, J. Y., Sizemore, G. M., Kladney, R., Zhang, J., Lehman, N. L., Ostrowski, M. C., Hong, B., Caligiuri, M., Yu, J., and Kaur, B. (2018) PTEN expression by an oncolytic herpesvirus directs T-cell mediated tumor clearance, Nat Commun 9, 5006.
Talasila, K. M., Soentgerath, A., Euskirchen, P., Rosland, G. V., Wang, J., Huszthy, P. C., Prestegarden, L., Skaftnesmo, K. O., Sakariassen, P. O., Eskilsson, E., Stieber, D., Keunen, O., Brekka, N., Moen, I., Nigro, J. M., Vintermyr, O. K., Lund-Johansen, M., Niclou, S., Mork, S. J., Enger, P. O., Bjerkvig, R., and Miletic, H. (2013) EGFR wild-type amplification and activation promote invasion and development of glioblastoma independent of angiogenesis, Acta neuropathologica 125, 683-698.
Lynch, T. J., Bell, D. W., Sordella, R., Gurubhagavatula, S., Okimoto, R. A., Brannigan, B. W., Harris, P. L., Haserlat, S. M., Supko, J. G., Haluska, F. G., Louis, D. N., Christiani, D. C., Settleman, J., and Haber, D. A. (2004) Activating mutations in the epidermal growth factor receptor underlying responsiveness of non-small-cell lung cancer to gefitinib, N Engl J Med 350, 2129-2139.
Paez, J. G., Janne, P. A., Lee, J. C., Tracy, S., Greulich, H., Gabriel, S., Herman, P., Kaye, F. J., Lindeman, N., Boggon, T. J., Naoki, K., Sasaki, H., Fujii, Y., Eck, M. J., Sellers, W. R., Johnson, B. E., and Meyerson, M. (2004) EGFR mutations in lung cancer: correlation with clinical response to gefitinib therapy, Science 304, 1497-1500.
Luo, J., Solimini, N. L., and Elledge, S. J. (2009) Principles of cancer therapy: oncogene and non-oncogene addiction, Cell 136, 823-837.
Sharma, S. V., Bell, D. W., Settleman, J., and Haber, D. A. (2007) Epidermal growth factor receptor mutations in lung cancer, Nat Rev Cancer 7, 169-181.
Chong, C. R., and Janne, P. A. (2013) The quest to overcome resistance to EGFR-targeted therapies in cancer, Nature medicine 19, 1389-1400.
Zhang, Z., Lee, J. C., Lin, L., Olivas, V., Au, V., LaFramboise, T., Abdel-Rahman, M., Wang, X., Levine, A. D., Rho, J. K., Choi, Y. J., Choi, C. M., Kim, S. W., Jang, S. J., Park, Y. S., Kim, W. S., Lee, D. H., Lee, J. S., Miller, V. A., Arcila, M., Ladanyi, M., Moonsamy, P., Sawyers, C., Boggon, T. J., Ma, P. C., Costa, C., Taron, M., Rosell, R., Halmos, B., and Bivona, T. G. (2012) Activation of the AXL kinase causes resistance to EGFR-targeted therapy in lung cancer, Nat Genet 44, 852-860.
Lee, H. J., Zhuang, G., Cao, Y., Du, P., Kim, H. J., and Settleman, J. (2014) Drug resistance via feedback activation of Stat3 in oncogene-addicted cancer cells, Cancer Cell 26, 207-221.
Ma, L., She, C., Shi, Q., Yin, Q., Ji, X., Wang, Y., Fan, Y., Kong, X., Li, P., Sun, Z., Zhang, X., Zhang, Z., Wang, J., Wang, T., Xu, Y., and Li, W. (2019) TNFalpha inhibitor C87 sensitizes EGFRvIII transfected glioblastoma cells to gefitinib by a concurrent blockade of TNFalpha signaling, Cancer Biol Med 16, 606-617.
Fallahi-Sichani, M., Moerke, N. J., Niepel, M., Zhang, T., Gray, N. S., and Sorger, P. K. (2015) Systematic analysis of BRAF(V600E) melanomas reveals a role for JNK/c-Jun pathway in adaptive resistance to drug-induced apoptosis, Mol Syst Biol 11, 797.
Fan, Z., Wirth, A. K., Chen, D., Wruck, C. J., Rauh, M., Buchfelder, M., and Savaskan, N. (2017) Nrf2-Keap1 pathway promotes cell proliferation and diminishes ferroptosis, Oncogenesis 6, e371.
Tsuchihashi, K., Okazaki, S., Ohmura, M., Ishikawa, M., Sampetrean, O., Onishi, N., Wakimoto, H., Yoshikawa, M., Seishima, R., Iwasaki, Y., Morikawa, T., Abe, S., Takao, A., Shimizu, M., Masuko, T., Nagane, M., Furnari, F. B., Akiyama, T., Suematsu, M., Baba, E., Akashi, K., Saya, H., and Nagano, O. (2016) The EGF Receptor Promotes the Malignant Potential of Glioma by Regulating Amino Acid Transport System xc(−), Cancer Res 76, 2954-2963.
Guo, D., Prins, R. M., Dang, J., Kuga, D., Iwanami, A., Soto, H., Lin, K. Y., Huang, T. T., Akhavan, D., Hock, M. B., Zhu, S., Kofman, A. A., Bensinger, S. J., Yong, W. H., Vinters, H. V., Horvath, S., Watson, A. D., Kuhn, J. G., Robins, H. I., Mehta, M. P., Wen, P. Y., DeAngelis, L. M., Prados, M. D., Mellinghoff, I. K., Cloughesy, T. F., and Mischel, P. S. (2009) EGFR signaling through an Akt-SREBP-1-dependent, rapamycin-resistant pathway sensitizes glioblastomas to antilipogenic therapy, Sci Signal 2, ra82.
Lu, K. V., Zhu, S., Cvrljevic, A., Huang, T. T., Sarkaria, S., Ahkavan, D., Dang, J., Dinca, E. B., Plaisier, S. B., Oderberg, I., Lee, Y., Chen, Z., Caldwell, J. S., Xie, Y., Loo, J. A., Seligson, D., Chakravari, A., Lee, F. Y., Weinmann, R., Cloughesy, T. F., Nelson, S. F., Bergers, G., Graeber, T., Furnari, F. B., James, C. D., Cavenee, W. K., Johns, T. G., and Mischel, P. S. (2009) Fyn and SRC are effectors of oncogenic epidermal growth factor receptor signaling in glioblastoma patients, Cancer Res 69, 6889-6898.
Puliyappadamba, V. T., Chakraborty, S., Chauncey, S. S., Li, L., Hatanpaa, K. J., Mickey, B., Noorani, S., Shu, H. K., Burma, S., Boothman, D. A., and Habib, A. A. (2013) Opposing Effect of EGFRWT on EGFRvIII-Mediated NF-kappaB Activation with RIP1 as a Cell Death Switch, Cell Rep 4, 764-775.
Sarkaria, J. N., Carlson, B. L., Schroeder, M. A., Grogan, P., Brown, P. D., Giannini, C., Ballman, K. V., Kitange, G. J., Guha, A., Pandita, A., and James, C. D. (2006) Use of an orthotopic xenograft model for assessing the effect of epidermal growth factor receptor amplification on glioblastoma radiation response, Clin Cancer Res 12, 2264-2271.
Estrada-Bernal, A., Lawler, S. E., Nowicki, M. O., Ray Chaudhury, A., and Van Brocklyn, J. R. (2011) The role of sphingosine kinase-1 in EGFRvIII-regulated growth and survival of glioblastoma cells, J Neurooncol 102, 353-366.
Carlson, B. L., Pokorny, J. L., Schroeder, M. A., and Sarkaria, J. N. (2011) Establishment, maintenance and in vitro and in vivo applications of primary human glioblastoma multiforme (GBM) xenograft models for translational biology studies and drug discovery, Curr Protoc Pharmacol Chapter 14, Unit 14 16.
McNeill, R. S., Vitucci, M., Wu, J., and Miller, C. R. (2015) Contemporary murine models in preclinical astrocytoma drug development, Neuro Oncol 17, 12-28.
Jaramillo, M. C., and Zhang, D. D. (2013) The emerging role of the Nrf2-Keap1 signaling pathway in cancer, Genes Dev 27, 2179-2191.
Romero, R., Sayin, V. I., Davidson, S. M., Bauer, M. R., Singh, S. X., LeBoeuf, S. E., Karakousi, T. R., Ellis, D. C., Bhutkar, A., Sanchez-Rivera, F. J., Subbaraj, L., Martinez, B., Bronson, R. T., Prigge, J. R., Schmidt, E. E., Thomas, C. J., Goparaju, C., Davies, A., Dolgalev, I., Heguy, A., Allaj, V., Poirier, J. T., Moreira, A. L., Rudin, C. M., Pass, H. I., Vander Heiden, M. G., Jacks, T., and Papagiannakopoulos, T. (2017) Keap1 loss promotes Kras-driven lung cancer and results in dependence on glutaminolysis, Nature medicine 23, 1362-1368.
Kerins, M. J., and Ooi, A. (2018) A catalogue of somatic NRF2 gain-of-function mutations in cancer, Sci Rep 8, 12846.
Ooi, A., Dykema, K., Ansari, A., Petillo, D., Snider, J., Kahnoski, R., Anema, J., Craig, D., Carpten, J., Teh, B. T., and Furge, K. A. (2013) CUL3 and NRF2 mutations confer an NRF2 activation phenotype in a sporadic form of papillary renal cell carcinoma, Cancer Res 73, 2044-2051.
Hayes, J. D., and McMahon, M. (2009) NRF2 and KEAP1 mutations: permanent activation of an adaptive response in cancer, Trends in biochemical sciences 34, 176-188.
Kim, Y. R., Oh, J. E., Kim, M. S., Kang, M. R., Park, S. W., Han, J. Y., Eom, H. S., Yoo, N. J., and Lee, S. H. (2010) Oncogenic NRF2 mutations in squamous cell carcinomas of oesophagus and skin, The Journal of pathology 220, 446-451.
Shibata, T., Ohta, T., Tong, K. I., Kokubu, A., Odogawa, R., Tsuta, K., Asamura, H., Yamamoto, M., and Hirohashi, S. (2008) Cancer related mutations in NRF2 impair its recognition by Keap1-Cul3 E3 ligase and promote malignancy, Proc Natl Acad Sci USA 105, 13568-13573.
Padmanabhan, B., Tong, K. I., Ohta, T., Nakamura, Y., Scharlock, M., Ohtsuji, M., Kang, M. I., Kobayashi, A., Yokoyama, S., and Yamamoto, M. (2006) Structural basis for defects of Keap1 activity provoked by its point mutations in lung cancer, Mol Cell 21, 689-700
Singh, A., Misra, V., Thimmulappa, R. K., Lee, H., Ames, S., Hoque, M. O., Herman, J. G., Baylin, S. B., Sidransky, D., Gabrielson, E., Brock, M. V., and Biswal, S. (2006) Dysfunctional KEAP1-NRF2 interaction in non-small-cell lung cancer, PLoS Med 3, e420.
Li, Q. K., Singh, A., Biswal, S., Askin, F., and Gabrielson, E. (2011) KEAP1 gene mutations and NRF2 activation are common in pulmonary papillary adenocarcinoma, J Hum Genet 56, 230-234.
Konstantinopoulos, P. A., Spentzos, D., Fountzilas, E., Francoeur, N., Sanisetty, S., Grammatikos, A. P., Hecht, J. L., and Cannistra, S. A. (2011) Keap1 mutations and Nrf2 pathway activation in epithelial ovarian cancer, Cancer Res 71, 5081-5089.
Solis, L. M., Behrens, C., Dong, W., Suraokar, M., Ozbum, N. C., Moran, C. A., Corvalan, A. H., Biswal, S., Swisher, S. G., Bekele, B. N., Minna, J. D., Stewart, D. J., and Wistuba, I I. (2010) Nrf2 and Keap1 abnormalities in non-small cell lung carcinoma and association with clinicopathologic features, Clin Cancer Res 16, 3743-3753.
Zhu, M., and Fahl, W. E. (2001) Functional characterization of transcription regulators that interact with the electrophile response element, Biochem Biophys Res Commun 289, 212-219.
Cui, Q., Wang, J. Q., Assaraf, Y. G., Ren, L., Gupta, P., Wei, L., Ashby, C. R., Jr., Yang, D. H., and Chen, Z. S. (2018) Modulating ROS to overcome multidrug resistance in cancer, Drug Resist Updat 41, 1-25.
Zhang, J., Wang, X., Vikash, V., Ye, Q., Wu, D., Liu, Y., and Dong, W. (2016) ROS and ROS-Mediated Cellular Signaling, Oxid Med Cell Longev 2016, 4350965.
Sabharwal, S. S., and Schumacker, P. T. (2014) Mitochondrial ROS in cancer: initiators, amplifiers or an Achilles' heel?, Nat Rev Cancer 14, 709-721.
Liou, G. Y., and Storz, P. (2010) Reactive oxygen species in cancer, Free Radic Res 44, 479-496.
Stupp, R., Mason, W. P., van den Bent, M. J., Weller, M., Fisher, B., Taphoom, M. J., Belanger, K., Brandes, A. A., Marosi, C., Bogdahn, U., Curschmann, J., Janzer, R. C., Ludwin, S. K., Gorlia, T., Allgeier, A., Lacombe, D., Cairncross, J. G., Eisenhauer, E., and Mirimanoff, R. O. (2005) Radiotherapy plus concomitant and adjuvant temozolomide for glioblastoma, N Engl J Med 352, 987-996.
Hegi, M. E., Diserens, A. C., Gorlia, T., Hamou, M. F., de Tribolet, N., Weller, M., Kros, J. M., Hainfellner, J. A., Mason, W., Mariani, L., Bromberg, J. E., Hau, P., Mirimanoff, R. O., Cairncross, J. G., Janzer, R. C., and Stupp, R. (2005) MGMT gene silencing and benefit from temozolomide in glioblastoma, N Engl J Med 352, 997-1003.
Hirst, T. C., Vesterinen, H. M., Sena, E. S., Egan, K. J., Macleod, M. R., and Whittle, I. R. (2013) Systematic review and meta-analysis of temozolomide in animal models of glioma: was clinical efficacy predicted?, Br J Cancer 108, 64-71.
Bansal, A., and Simon, M. C. (2018) Glutathione metabolism in cancer progression and treatment resistance, J Cell Biol 217, 2291-2298.
Chen, H. H., and Kuo, M. T. (2010) Role of glutathione in the regulation of Cisplatin resistance in cancer chemotherapy, Met Based Drugs 2010.
Frank, O., Brors, B., Fabarius, A., Li, L., Haak, M., Merk, S., Schwindel, U., Zheng, C., Muller, M. C., Gretz, N., Hehlmann, R., Hochhaus, A., and Seifarth, W. (2006) Gene expression signature of primary imatinib-resistant chronic myeloid leukemia patients, Leukemia 20, 1400-1407.
Jia, Y., Zhang, W., Liu, H., Peng, L., Yang, Z., and Lou, J. (2012) Inhibition of glutathione synthesis reverses Kruppel-like factor 4-mediated cisplatin resistance, Cancer Chemother Pharmacol 69, 377-385.
Jia, Y., Zhang, C., Zhou, L., Xu, H., Shi, Y., and Tong, Z. (2015) Micheliolide overcomes KLF4-mediated cisplatin resistance in breast cancer cells by downregulating glutathione, Onco Targets Ther 8, 2319-2327.
Starheim, K. K., Holien, T., Misund, K., Johansson, I., Baranowska, K. A., Sponaas, A. M., Hella, H., Buene, G., Waage, A., Sundan, A., and Bjorkoy, G. (2016) Intracellular glutathione determines bortezomib cytotoxicity in multiple myeloma cells, Blood Cancer J 6, e446.
Wang, Z., Liang, S., Lian, X., Liu, L., Zhao, S., Xuan, Q., Guo, L., Liu, H., Yang, Y., Dong, T., Liu, Y., Liu, Z., and Zhang, Q. (2015) Identification of proteins responsible for adriamycin resistance in breast cancer cells using proteomics analysis, Sci Rep 5, 9301.
Zhu, Z., Du, S., Du, Y., Ren, J., Ying, G., and Yan, Z. (2018) Glutathione reductase mediates drug resistance in glioblastoma cells by regulating redox homeostasis, J Neurochem 144, 93-104.
Yuan, M., Breitkopf, S. B., Yang, X., and Asara, J. M. (2012) A positive/negative ion-switching, targeted mass spectrometry-based metabolomics platform for bodily fluids, cells, and fresh and fixed tissue, Nat Protoc 7, 872-881.
Zitka, O., Skalickova, S., Gumulec, J., Masarik, M., Adam, V., Hubalek, J., Trnkova, L., Kruseova, J., Eckschlager, T., and Kizek, R. (2012) Redox status expressed as GSH:GSSG ratio as a marker for oxidative stress in paediatric tumour patients, Oncol Lett 4, 1247-1253.
Mohell, N., Alfredsson, J., Fransson, A., Uustalu, M., Bystrom, S., Gullbo, J., Hallberg, A., Bykov, V. J., Bjorklund, U., and Wiman, K. G. (2015) APR-246 overcomes resistance to cisplatin and doxorubicin in ovarian cancer cells, Cell Death Dis 6, e1794.
Liu, D. S., Read, M., Cullinane, C., Azar, W. J., Fennell, C. M., Montgomery, K. G., Haupt, S., Haupt, Y., Wiman, K. G., Duong, C. P., Clemons, N. J., and Phillips, W. A. (2015) APR-246 potently inhibits tumour growth and overcomes chemoresistance in preclinical models of oesophageal adenocarcinoma, Gut 64, 1506-1516.
Pelosof, L., Yerram, S., Armstrong, T., Chu, N., Danilova, L., Yanagisawa, B., Hidalgo, M., Azad, N., and Herman, J. G. (2017) GPX3 promoter methylation predicts platinum sensitivity in colorectal cancer, Epigenetics 12, 540-550.
Worley, B. L., Kim, Y. S., Mardini, J., Zaman, R., Leon, K. E., Vallur, P. G., Nduwumwami, A., Warrick, J. I., Timmins, P. F., Kesterson, J. P., Phaeton, R., Lee, N. Y., Walter, V., Endres, L., Mythreye, K., Aird, K. M., and Hempel, N. (2018) GPx3 supports ovarian cancer progression by manipulating the extracellular redox environment, Redox Biol.
Hall, M. D., Marshall, T. S., Kwit, A. D., Miller Jenkins, L. M., Dulcey, A. E., Madigan, J. P., Pluchino, K. M., Goldsborough, A. S., Brimacombe, K. R., Griffiths, G. L., and Gottesman, M. M. (2014) Inhibition of glutathione peroxidase mediates the collateral sensitivity of multidrug-resistant cells to tiopronin, J Biol Chem 289, 21473-21489.
Chaudiere, J., Wilhelmsen, E. C., and Tappel, A. L. (1984) Mechanism of selenium-glutathione peroxidase and its inhibition by mercaptocarboxylic acids and other mercaptans, J Biol Chem 259, 1043-1050.
Pathania, S., Bhatia, R., Baldi, A., Singh, R., and Rawal, R. K. (2018) Drug metabolizing enzymes and their inhibitors' role in cancer resistance, Biomed Pharmacother 105, 53-65.
Chatterjee, A., and Gupta, S. (2018) The multifaceted role of glutathione S-transferases in cancer, Cancer Lett 433, 33-42.
Chen, Z., Shi, T., Zhang, L., Zhu, P., Deng, M., Huang, C., Hu, T., Jiang, L., and Li, J. (2016) Mammalian drug efflux transporters of the ATP binding cassette (ABC) family in multidrug resistance: A review of the past decade, Cancer Lett 370, 153-164.
Fletcher, J. I., Williams, R. T., Henderson, M. J., Norris, M. D., and Haber, M. (2016) ABC transporters as mediators of drug resistance and contributors to cancer cell biology, Drug Resist Updat 26, 1-9.
Hedditch, E. L., Gao, B., Russell, A. J., Lu, Y., Emmanuel, C., Beesley, J., Johnatty, S. E., Chen, X., Harnett, P., George, J., Australian Ovarian Cancer Study, G., Williams, R. T., Flemming, C., Lambrechts, D., Despierre, E., Lambrechts, S., Vergote, I., Karlan, B., Lester, J., Orsulic, S., Walsh, C., Fasching, P., Beckmann, M. W., Ekici, A. B., Hein, A., Matsuo, K., Hosono, S., Nakanishi, T., Yatabe, Y., Pejovic, T., Bean, Y., Heitz, F., Harter, P., du Bois, A., Schwaab, I., Hogdall, E., Kjaer, S. K., Jensen, A., Hogdall, C., Lundvall, L., Engelholm, S. A., Brown, B., Flanagan, J., Metcalf, M. D., Siddiqui, N., Sellers, T., Fridley, B., Cunningham, J., Schildkraut, J., Iversen, E., Weber, R. P., Berchuck, A., Goode, E., Bowtell, D. D., Chenevix-Trench, G., deFazio, A., Norris, M. D., MacGregor, S., Haber, M., and Henderson, M. J. (2014) ABCA transporter gene expression and poor outcome in epithelial ovarian cancer, Journal of the National Cancer Institute 106.
Yamada, A., Ishikawa, T., Ota, I., Kimura, M., Shimizu, D., Tanabe, M., Chishima, T., Sasaki, T., Ichikawa, Y., Morita, S., Yoshiura, K., Takabe, K., and Endo, I. (2013) High expression of ATP-binding cassette transporter ABCC11 in breast tumors is associated with aggressive subtypes and low disease-free survival, Breast cancer research and treatment 137, 773-782.
Bronger, H., Konig, J., Kopplow, K., Steiner, H. H., Ahmadi, R., Herold-Mende, C., Keppler, D., and Nies, A. T. (2005) ABCC drug efflux pumps and organic anion uptake transporters in human gliomas and the blood-tumor barrier, Cancer Res 65, 11419-11428.
Sleire, L., Skeie, B. S., Netland, I. A., Forde, H. E., Dodoo, E., Selheim, F., Leiss, L., Heggdal, J. I., Pedersen, P. H., Wang, J., and Enger, P. O. (2015) Drug repurposing: sulfasalazine sensitizes gliomas to gamma knife radiosurgery by blocking cystine uptake through system Xc-, leading to glutathione depletion, Oncogene 34, 5951-5959.
Chen, L., Li, X., Liu, L., Yu, B., Xue, Y., and Liu, Y. (2015) Erastin sensitizes glioblastoma cells to temozolomide by restraining xCT and cystathionine-gamma-lyase function, Oncology reports 33, 1465-1474.
Dinkova-Kostova, A. T., and Talalay, P. (2010) NAD(P)H:quinone acceptor oxidoreductase 1 (NQO1), a multifunctional antioxidant enzyme and exceptionally versatile cytoprotector, Arch Biochem Biophys 501, 116-123.
Dinkova-Kostova, A. T., Holtzclaw, W. D., Cole, R. N., Itoh, K., Wakabayashi, N., Katoh, Y., Yamamoto, M., and Talalay, P. (2002) Direct evidence that sulfhydryl groups of Keap1 are the sensors regulating induction of phase 2 enzymes that protect against carcinogens and oxidants, Proc Natl Acad Sci USA 99, 11908-11913.
Puchalski, R. B., Shah, N., Miller, J., Dalley, R., Nomura, S. R., Yoon, J. G., Smith, K. A., Lankerovich, M., Bertagnolli, D., Bickley, K., Boe, A. F., Brouner, K., Butler, S., Caldejon, S., Chapin, M., Datta, S., Dee, N., Desta, T., Dolbeare, T., Dotson, N., Ebbert, A., Feng, D., Feng, X., Fisher, M., Gee, G., Goldy, J., Gourley, L., Gregor, B. W., Gu, G., Hejazinia, N., Hohmann, J., Hothi, P., Howard, R., Joines, K., Kriedberg, A., Kuan, L., Lau, C., Lee, F., Lee, H., Lemon, T., Long, F., Mastan, N., Mott, E., Murthy, C., Ngo, K., Olson, E., Reding, M., Riley, Z., Rosen, D., Sandman, D., Shapovalova, N., Slaughterbeck, C. R., Sodt, A., Stockdale, G., Szafer, A., Wakeman, W., Wohnoutka, P. E., White, S. J., Marsh, D., Rostomily, R. C., Ng, L., Dang, C., Jones, A., Keogh, B., Gittleman, H. R., Barnholtz-Sloan, J. S., Cimino, P. J., Uppin, M. S., Keene, C. D., Farrokhi, F. R., Lathia, J. D., Berens, M. E., Iavarone, A., Bernard, A., Lein, E., Phillips, J. W., Rostad, S. W., Cobbs, C., Hawrylycz, M. J., and Foltz, G. D. (2018) An anatomic transcriptional atlas of human glioblastoma, Science 360, 660-663.
Chakraborty, S., Li, L., Tang, H., Xie, Y., Puliyappadamba, V. T., Raisanen, J., Burma, S., Boothman, D. A., Cochran, B., Wu, J., and Habib, A. A. (2013) Cytoplasmic TRADD Confers a Worse Prognosis in Glioblastoma, Neoplasia 15, 888-897.
Park, S., Hatanpaa, K. J., Xie, Y., Mickey, B. E., Madden, C. J., Raisanen, J. M., Ramnarain, D. B., Xiao, G., Saha, D., Boothman, D. A., Zhao, D., Bachoo, R. M., Pieper, R. O., and Habib, A. A. (2009) The receptor interacting protein 1 inhibits p53 induction through NF-kappaB activation and confers a worse prognosis in glioblastoma, Cancer Res 69, 2809-2816.
Fan, Q. W., Specht, K. M., Zhang, C., Goldenberg, D. D., Shokat, K. M., and Weiss, W. A. (2003) Combinatorial efficacy achieved through two-point blockade within a signaling pathway—a chemical genetic approach, Cancer Res 63, 8930-8938.
Fan, Q. W., Knight, Z. A., Goldenberg, D. D., Yu, W., Mostov, K. E., Stokoe, D., Shokat, K. M., and Weiss, W. A. (2006) A dual PI3 kinase/mTOR inhibitor reveals emergent efficacy in glioma, Cancer Cell 9, 341-349.
Kansanen, E., Kuosmanen, S. M., Leinonen, H., and Levonen, A. L. (2013) The Keap1-Nrf2 pathway: Mechanisms of activation and dysregulation in cancer, Redox Biol 1, 45-49
Orcutt, K. P., Parsons, A. D., Sibenaller, Z. A., Scarbrough, P. M., Zhu, Y., Sobhakumari, A., Wilke, W. W., Kalen, A. L., Goswami, P., Miller, F. J., Jr., Spitz, D. R., and Simons, A. L. (2011) Erlotinib-mediated inhibition of EGFR signaling induces metabolic oxidative stress through NOX4, Cancer Res 71, 3932-3940.
Qian, X., Li, J., Ding, J., Wang, Z., Zhang, W., and Hu, G. (2009) Erlotinib activates mitochondrial death pathways related to the production of reactive oxygen species in the human non-small cell lung cancer cell line A549, Clin Exp Pharmacol Physiol 36, 487-494.
Liou, G. Y., and Storz, P. (2015) Detecting reactive oxygen species by immunohistochemistry, Methods Mol Biol 1292, 97-104.
Bryan, H. K., Olayanju, A., Goldring, C. E., and Park, B. K. (2013) The Nrf2 cell defence pathway: Keap1-dependent and -independent mechanisms of regulation, Biochem Pharmacol 85, 705-717.
Fourquet, S., Guerois, R., Biard, D., and Toledano, M. B. (2010) Activation of NRF2 by nitrosative agents and H₂O₂ involves KEAP1 disulfide formation, J Biol Chem 285, 8463-8471.
Komatsu, M., Kurokawa, H., Waguri, S., Taguchi, K., Kobayashi, A., Ichimura, Y., Sou, Y. S., Ueno, I., Sakamoto, A., Tong, K. I., Kim, M., Nishito, Y., Iemura, S., Natsume, T., Ueno, T., Kominami, E., Motohashi, H., Tanaka, K., and Yamamoto, M. (2010) The selective autophagy substrate p62 activates the stress responsive transcription factor Nrf2 through inactivation of Keap1, Nat Cell Biol 12, 213-223.
Bae, S. H., Sung, S. H., Oh, S. Y., Lim, J. M., Lee, S. K., Park, Y. N., Lee, H. E., Kang, D., and Rhee, S. G. (2013) Sestrins activate Nrf2 by promoting p62-dependent autophagic degradation of Keap1 and prevent oxidative liver damage, Cell Metab 17, 73-84.
Ausubel, F. M. (1987) Current protocols in molecular biology, Published by Greene Pub. Associates and Wiley-Interscience: J. Wiley, New York.
Ichimura, Y., Waguri, S., Sou, Y. S., Kageyama, S., Hasegawa, J., Ishimura, R., Saito, T., Yang, Y., Kouno, T., Fukutomi, T., Hoshii, T., Hirao, A., Takagi, K., Mizushima, T., Motohashi, H., Lee, M. S., Yoshimori, T., Tanaka, K., Yamamoto, M., and Komatsu, M. (2013) Phosphorylation of p62 activates the Keap1-Nrf2 pathway during selective autophagy, Mol Cell 51, 618-631.
Hast, B. E., Goldfarb, D., Mulvaney, K. M., Hast, M. A., Siesser, P. F., Yan, F., Hayes, D. N., and Major, M. B. (2013) Proteomic analysis of ubiquitin ligase KEAP1 reveals associated proteins that inhibit NRF2 ubiquitination, Cancer Res 73, 2199-2210.
Lu, K., Alcivar, A. L., Ma, J., Foo, T. K., Zywea, S., Mandi, A., Huo, Y., Kensler, T. W., Gatza, M. L., and Xia, B. (2017) NRF2 Induction Supporting Breast Cancer Cell Survival Is Enabled by Oxidative Stress-Induced DPP3-KEAP1 Interaction, Cancer Res 77, 2881-2892.
Gorrini, C., Baniasadi, P. S., Harris, I. S., Silvester, J., Inoue, S., Snow, B., Joshi, P. A., Wakeham, A., Molyneux, S. D., Martin, B., Bouwman, P., Cescon, D. W., Elia, A. J., Winterton-Perks, Z., Cruickshank, J., Brenner, D., Tseng, A., Musgrave, M., Berman, H. K., Khokha, R., Jonkers, J., Mak, T. W., and Gauthier, M. L. (2013) BRCA1 interacts with Nrf2 to regulate antioxidant signaling and cell survival, J Exp Med 210, 1529-1544.
Rasmussen, R. D., Gajjar, M. K., Tuckova, L., Jensen, K. E., Maya-Mendoza, A., Holst, C. B., Mollgaard, K., Rasmussen, J. S., Brennum, J., Bartek, J., Jr., Syrucek, M., Sedlakova, E., Andersen, K. K., Frederiksen, M. H., Bartek, J., and Hamerlik, P. (2016) BRCA1-regulated RRM2 expression protects glioblastoma cells from endogenous replication stress and promotes tumorigenicity, Nat Commun 7, 13398.
Camp, N. D., James, R. G., Dawson, D. W., Yan, F., Davison, J. M., Houck, S. A., Tang, X., Zheng, N., Major, M. B., and Moon, R. T. (2012) Wilms tumor gene on X chromosome (WTX) inhibits degradation of NRF2 protein through competitive binding to KEAP1 protein, J Biol Chem 287, 6539-6550.
Karapetian, R. N., Evstafieva, A. G., Abaeva, I. S., Chichkova, N. V., Filonov, G. S., Rubtsov, Y. P., Sukhacheva, E. A., Melnikov, S. V., Schneider, U., Wanker, E. E., and Vartapetian, A. B. (2005) Nuclear oncoprotein prothymosin alpha is a partner of Keap1: implications for expression of oxidative stress-protecting genes, Mol Cell Biol 25, 1089-1099.
Padmanabhan, B., Nakamura, Y., and Yokoyama, S. (2008) Structural analysis of the complex of Keap1 with a prothymosin alpha peptide, Acta Crystallogr Sect F Struct Biol Cryst Commun 64, 233-238.
Ma, J., Cai, H., Wu, T., Sobhian, B., Huo, Y., Alcivar, A., Mehta, M., Cheung, K. L., Ganesan, S., Kong, A. N., Zhang, D. D., and Xia, B. (2012) PALB2 interacts with KEAP1 to promote NRF2 nuclear accumulation and function, Mol Cell Biol 32, 1506-1517.
O'Reilly, M. A. (2005) Redox activation of p21Cip1/WAF1/Sdi1: a multifunctional regulator of cell survival and death, Antioxid Redox Signal 7, 108-118.
Chen, W., Sun, Z., Wang, X. J., Jiang, T., Huang, Z., Fang, D., and Zhang, D. D. (2009) Direct interaction between Nrf2 and p21(Cip1/WAF1) upregulates the Nrf2-mediated antioxidant response, Mol Cell 34, 663-673.
Raizer, J. J., Abrey, L. E., Lassman, A. B., Chang, S. M., Lamborn, K. R., Kuhn, J. G., Yung, W. K., Gilbert, M. R., Aldape, K. A., Wen, P. Y., Fine, H. A., Mehta, M., Deangelis, L. M., Lieberman, F., Cloughesy, T. F., Robins, H. I., Dancey, J., Prados, M. D., and North American Brain Tumor, C. (2010) A phase II trial of erlotinib in patients with recurrent malignant gliomas and nonprogressive glioblastoma multiforme postradiation therapy, Neuro Oncol 12, 95-103.
Sivanand, S., Pena-Llopis, S., Zhao, H., Kucejova, B., Spence, P., Pavia-Jimenez, A., Yamasaki, T., McBride, D. J., Gillen, J., Wolff, N. C., Morlock, L., Lotan, Y., Raj, G. V., Sagalowsky, A., Margulis, V., Cadeddu, J. A., Ross, M. T., Bentley, D. R., Kabbani, W., Xie, X. J., Kapur, P., Williams, N. S., and Brugarolas, J. (2012) A validated tumorgraft model reveals activity of dovitinib against renal cell carcinoma, Sci Transl Med 4, 137ra175.
Schnell, D., Buschke, S., Fuchs, H., Gansser, D., Goeldner, R. G., Uttenreuther-Fischer, M., Stopfer, P., Wind, S., Petersen-Sylla, M., Halabi, A., and Koenen, R. (2014) Pharmacokinetics of afatinib in subjects with mild or moderate hepatic impairment, Cancer Chemother Pharmacol 74, 267-275.
Wind, S., Schnell, D., Ebner, T., Freiwald, M., and Stopfer, P. (2017) Clinical Pharmacokinetics and Pharmacodynamics of Afatinib, Clin Pharmacokinet 56, 235-250.
Zhang, S. R., Zhu, L. C., Jiang, Y. P., Zhang, J., Xu, R. J., Xu, Y. S., Xia, B., and Ma, S. L. (2017) Efficacy of afatinib, an irreversible ErbB family blocker, in the treatment of intracerebral metastases of non-small cell lung cancer in mice, Acta Pharmacol Sin 38, 233-240.
Xie, Y., Zhu, S., Song, X., Sun, X., Fan, Y., Liu, J., Zhong, M., Yuan, H., Zhang, L., Billiar, T. R., Lotze, M. T., Zeh, H. J., 3rd, Kang, R., Kroemer, G., and Tang, D. (2017) The Tumor Suppressor p53 Limits Ferroptosis by Blocking DPP4 Activity, Cell Rep 20, 1692-1704.
Huo, H., Zhou, Z., Qin, J., Liu, W., Wang, B., and Gu, Y. (2016) Erastin Disrupts Mitochondrial Permeability Transition Pore (mPTP) and Induces Apoptotic Death of Colorectal Cancer Cells, PLoS One 11, e0154605.
Chung, W. J., and Sontheimer, H. (2009) Sulfasalazine inhibits the growth of primary brain tumors independent of nuclear factor-kappaB, J Neurochem 110, 182-193.
Gu, G. Z., Xia, H. M., Pang, Z. Q., Liu, Z. Y., Jiang, X. G., and Chen, J. (2011) Determination of sulphasalazine and its main metabolite sulphapyridine and 5-aminosalicylic acid in human plasma by liquid chromatography/tandem mass spectrometry and its application to a pharmacokinetic study, Journal of chromatography. B, Analytical technologies in the biomedical and life sciences 879, 449-456.
Zheng, W., Winter, S. M., Mayersohn, M., Bishop, J. B., and Sipes, I. G. (1993) Toxicokinetics of sulfasalazine (salicylazosulfapyridine) and its metabolites in B6C3F1 mice, Drug Metab Dispos 21, 1091-1097.
Bhatt, N. B., Barau, C., Amin, A., Baudin, E., Meggi, B., Silva, C., Furlan, V., Grinsztejn, B., Barrail-Tran, A., Bonnet, M., Taburet, A. M., and Group, A. C. S. (2014) Pharmacokinetics of rifampin and isoniazid in tuberculosis-HIV-coinfected patients receiving nevirapine- or efavirenz-based antiretroviral treatment, Antimicrob Agents Chemother 58, 3182-3190.
Almeida, D., Nuermberger, E., Tasneen, R., Rosenthal, I., Tyagi, S., Williams, K., Peloquin, C., and Grosset, J. (2009) Paradoxical effect of isoniazid on the activity of rifampin-pyrazinamide combination in a mouse model of tuberculosis, Antimicrob Agents Chemother 53, 4178-4184.
Singh, A., Venkannagari, S., Oh, K. H., Zhang, Y. Q., Rohde, J. M., Liu, L., Nimmagadda, S., Sudini, K., Brimacombe, K. R., Gajghate, S., Ma, J., Wang, A., Xu, X., Shahane, S. A., Xia, M., Woo, J., Mensah, G. A., Wang, Z., Ferrer, M., Gabrielson, E., Li, Z., Rastinejad, F., Shen, M., Boxer, M. B., and Biswal, S. (2016) Small Molecule Inhibitor of NRF2 Selectively Intervenes Therapeutic Resistance in KEAP1-Deficient NSCLC Tumors, ACS chemical biology 11, 3214-3225.
Bardella, C., Olivero, M., Lorenzato, A., Geuna, M., Adam, J., O'Flaherty, L., Rustin, P., Tomlinson, I., Pollard, P. J., and Di Renzo, M. F. (2012) Cells lacking the fumarase tumor suppressor are protected from apoptosis through a hypoxia-inducible factor-independent, AMPK-dependent mechanism, Mol Cell Biol 32, 3081-3094.
Bansal, H., Seifert, T., Bachier, C., Rao, M., Tomlinson, G., Iyer, S. P., and Bansal, S. (2012) The transcription factor Wilms tumor 1 confers resistance in myeloid leukemia cells against the proapoptotic therapeutic agent TRAIL (tumor necrosis factor alpha-related apoptosis-inducing ligand) by regulating the antiapoptotic protein Bcl-xL, J Biol Chem 287, 32875-32880.
Tang, J., Ip, J. P., Ye, T., Ng, Y. P., Yung, W. H., Wu, Z., Fang, W., Fu, A. K., and Ip, N. Y. (2014) Cdk5-dependent Mst3 phosphorylation and activity regulate neuronal migration through RhoA inhibition, J Neurosci 34, 7425-7436.
Lopez-Bertoni, H., Lal, B., Michelson, N., Guerrero-Cazares, H., Quinones-Hinojosa, A., Li, Y., and Laterra, J. (2016) Epigenetic modulation of a miR-296-5p:HMGA1 axis regulates Sox2 expression and glioblastoma stem cells, Oncogene 35, 4903-4913.
Gonzalez-Juarrero, M., Woolhiser, L. K., Brooks, E., DeGroote, M. A., and Lenaerts, A. J. (2012) Mouse model for efficacy testing of antituberculosis agents via intrapulmonary delivery, Antimicrob Agents Chemother 56, 3957-3959.
Dickinson, J., Guy, A., and Mitchison, D. A. (1992) Bioavailability of rifampin in experimental murine tuberculosis, Antimicrob Agents Chemother 36, 2066-2067.
Gupta, S. K., Kizilbash, S. H., Carlson, B. L., Mladek, A. C., Boakye-Agyeman, F., Bakken, K. K., Pokorny, J. L., Schroeder, M. A., Decker, P. A., Cen, L., Eckel-Passow, J. E., Sarkar, G., Ballman, K. V., Reid, J. M., Jenkins, R. B., Verhaak, R. G., Sulman, E. P., Kitange, G. J., and Sarkaria, J. N. (2016) Delineation of MGMT Hypermethylation as a Biomarker for Veliparib-Mediated Temozolomide-Sensitizing Therapy of Glioblastoma, Journal of the National Cancer Institute 108.
Shah, B. K., Bista, A., and Sharma, S. (2016) Survival Trends in Elderly Patients with Glioblastoma in the United States: a Population-based Study, Anticancer Res 36, 4883-4886.
Rusthoven, C. G., Koshy, M., Sher, D. J., Ney, D. E., Gaspar, L. E., Jones, B. L., Karam, S. D., Amini, A., Ormond, D. R., Youssef, A. S., and Kavanagh, B. D. (2016) Combined-Modality Therapy With Radiation and Chemotherapy for Elderly Patients With Glioblastoma in the Temozolomide Era: A National Cancer Database Analysis, JAMA Neurol 73, 821-828.
Russell, L., Bolyard, C., Banasavadi-Siddegowda, Y., Weiss, A., Zhang, J., Shakya, R., Powell, K., and Kaur, B. (2017) Sex as a biological variable in response to temozolomide, Neuro Oncol.
Zhou, H., Luby-Phelps, K., Mickey, B. E., Habib, A. A., Mason, R. P., and Zhao, D. (2009) Dynamic near-infrared optical imaging of 2-deoxyglucose uptake by intracranial glioma of athymic mice, PLoS One 4, e8051.
Gao Guo, K. G., Sonia Ali, Neha Ali, Shahzad Shallwani, Kimmo J. Hatanpaa, Edward Pan, Bruce Mickey, Sandeep Burma, David Wang, Santosh Kesari, Jann Sarkaria, Dawen Zhao and Amyn A. Habib. (2017) A TNF-JNK-Axl-ERK signaling axis mediates primary resistance to EGFR inhibition in glioblastoma, Nature Neuroscience In press.
Ji, X., Qian, J., Rahman, S. M. J., Siska, P. J., Zou, Y., Harris, B. K., Hoeksema, M. D., Trenary, I. A., Heidi, C., Eisenberg, R., Rathmell, J. C., Young, J. D., and Massion, P. P. (2018) xCT (SLC7A11)-mediated metabolic reprogramming promotes non-small cell lung cancer progression, Oncogene 37, 5007-5019.
Shukla, K., Thomas, A. G., Ferraris, D. V., Hin, N., Sattler, R., Alt, J., Rojas, C., Slusher, B. S., and Tsukamoto, T. (2011) Inhibition of xc(−) transporter-mediated cystine uptake by sulfasalazine analogs, Bioorg Med Chem Lett 21, 6184-6187.
Chung, W. J., Lyons, S. A., Nelson, G. M., Hamza, H., Gladson, C. L., Gillespie, G. Y., and Sontheimer, H. (2005) Inhibition of cystine uptake disrupts the growth of primary brain tumors, J Neurosci 25, 7101-7110.
Sontheimer, H., and Bridges, R. J. (2012) Sulfasalazine for brain cancer fits, Expert Opin Investig Drugs 21, 575-578.
Sato, M., Kusumi, R., Hamashima, S., Kobayashi, S., Sasaki, S., Komiyama, Y., Izumikawa, T., Conrad, M., Bannai, S., and Sato, H. (2018) The ferroptosis inducer erastin irreversibly inhibits system xc- and synergizes with cisplatin to increase cisplatin's cytotoxicity in cancer cells, Sci Rep 8, 968.
Li, X., Lin, Z., Zhang, B., Guo, L., Liu, S., Li, H., Zhang, J., and Ye, Q. (2016) beta-elemene sensitizes hepatocellular carcinoma cells to oxaliplatin by preventing oxaliplatin-induced degradation of copper transporter 1, Sci Rep 6, 21010.
Nair, A. B., and Jacob, S. (2016) A simple practice guide for dose conversion between animals and human, J Basic Clin Pharm 7, 27-31.

### G. EXAMPLES

Here, it is proposed that EGFR inhibition may not work in GBM because an adaptive survival mechanism triggered by EGFR inhibition negates its effect. A combined inhibition of EGFR+adaptive response either unmasks a requirement for EGFR signaling for survival and/or sets up synthetic lethal conditions. It was previously reported that a TNF-driven adaptive response mediates primary resistance to EGFR inhibition in GBM and lung cancer. The new preliminary data derived from transcriptome analysis indicate that the adaptive response to EGFR inhibition is multifaceted. Nrf2 signaling is herein identified as a TNF-independent pathway of central importance in the adaptive response to EGFR inhibition in EGFRwt and/or EGFRvIII expressing GBMs. Cancers with high Nrf2 levels are associated with chemotherapy and radiation therapy resistance and with a poor prognosis. The Nrf2 network activation drives multiple facets of gliomagenesis and treatment resistance and confers a worse prognosis. Without wishing to be bound by theory, the central hypothesis is that primary resistance to EGFR inhibition in GBM is mediated by an adaptive survival mechanism triggered by TKI exposure that involves activation of Nrf2 and that a combined inhibition of EGFR and Nrf2 is an effective treatment for the majority of GBMs. To examine this hypothesis, the following specific aims are proposed: (1) To elucidate the effector mechanisms downstream of Nrf2 activation that mediate resistance to EGFR inhibition. It is hypothesized that EGFR-inhibition induced Nrf2 activation leads to transcription of specific genes that mediate resistance to EGFR inhibition. Activation of these pathways is examined in experimental models as well as data from tumor tissue. (2) To elucidate the mechanism of EGFR TKI-induced Nrf2 activation. It is hypothesized that EGFR inhibition in GBM results in activation of Nrf2, a master regulator of cellular stress homeostasis. It is proposed that EGFR inhibition induced Nrf2 activation results from both canonical and non-canonical mechanisms. (3) To examine the biological effect of a combined inhibition of EGFR and Nrf2 or Nrf2 downstream targets in preclinical mouse orthotopic models. It is hypothesized that interruption of the adaptive homeostatic pro-survival Nrf2 signaling triggered by EGFR inhibition will transform GBMs with primary resistance into cancers that can be effectively treated by EGFR inhibition.

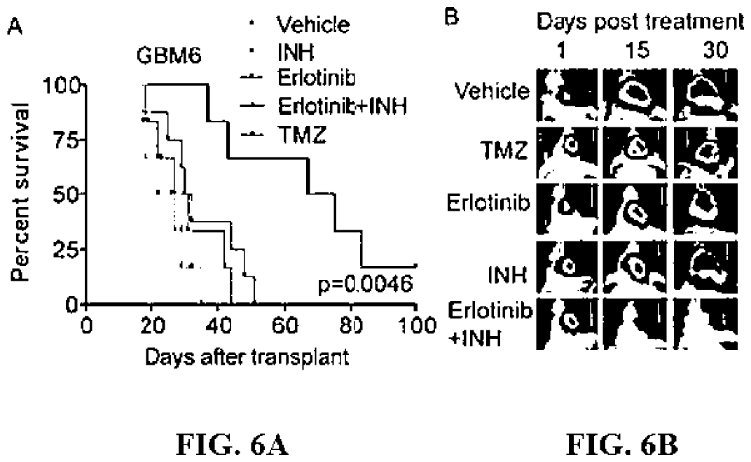

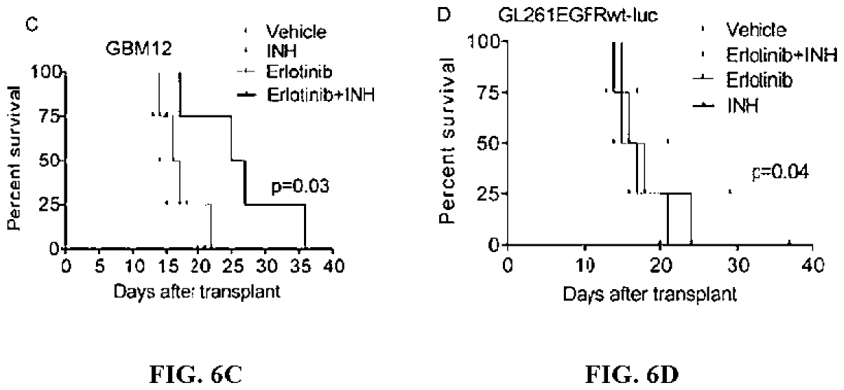

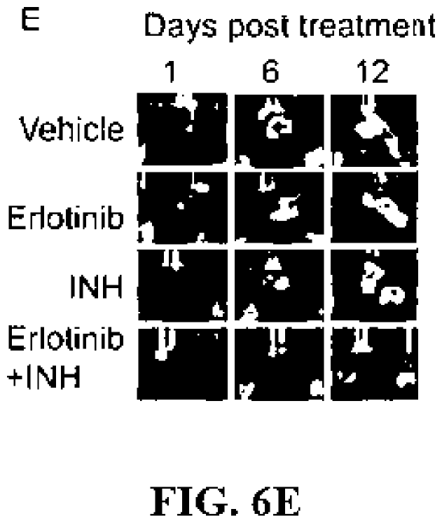

In addition, preliminary data showing that a combination of EGFR TKI plus isoniazid (INH) is highly effective in multiple preclinical GBM models (FIG. 6A-E). INH is a known Nrf2 inhibitor, is highly CNS penetrable, and has been widely used for tuberculous meningitis. Thus, the work proposed herein has a clear path to translation in GBM.

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

The Examples are provided herein to illustrate the invention, and should not be construed as limiting the invention in any way. Examples are provided herein to illustrate the invention and should not be construed as limiting the invention in any way.

1. Combined Inhibition of EGFR and Nrf2 in the Treatment of Malignant Glioma

Inhibition of specific signals in cancer cells leads to a feedback mediated escape from pathway inhibition by reprogramming of signaling pathways that lead to a resumption of previously suppressed signals or activation of alternative functionally similar signals. Resistance to targeted treatment is a formidable obstacle in GBM and includes resistance to EGFR inhibition and VEGF-A inhibition using bevacizumab. Immunotherapy has thus far failed, although interesting experimental approaches are under investigation. EGFR gene amplification is found in the classical subtype of GBM, and is detected in 40-50% of GBMs. EGFR can be detected in 81% of GBM, with strong expression (>75% of tumor cells express EGFR) in about 60% of GBM. Furthermore, a number of experimental studies have demonstrated a role for EGFR signaling as an oncogenic driver in experimental models suggesting that the EGFR is an attractive target in GBM.

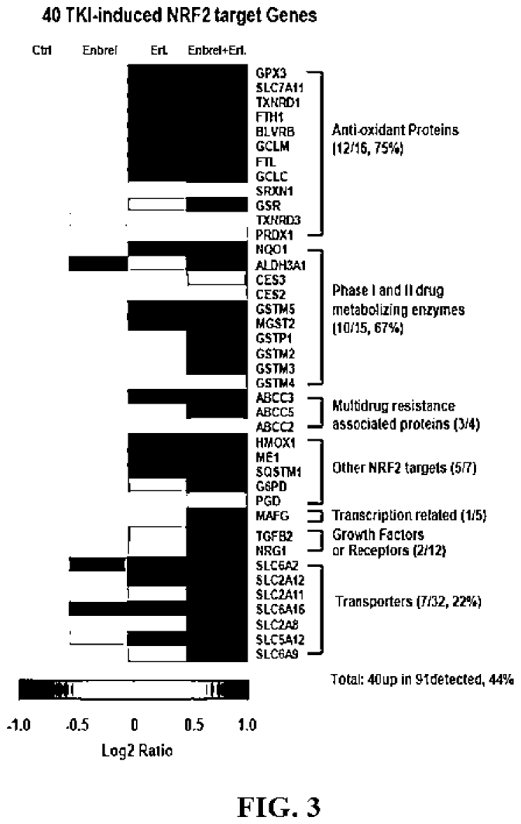

Although the major signaling pathways that drive the malignant phenotype in GBM are known, targeted treatment has not been successful. Thus, resistance to targeted inhibition has emerged as perhaps the most important barrier to effective treatment. In GBM, numerous clinical trials have indicated that EGFR inhibition is not effective in GBM. Lung cancers with activating EGFR mutations exhibit a dramatic initial clinical response, but this is followed by the inevitable development of secondary resistance to EGFR inhibition by mechanisms that include the T790M secondary EGFR mutation and activation of Met or Axl, or rapid feedback loops resulting in STAT3 or TNF activation. Studies of EGFR inhibition in GBM have focused primarily on delayed mechanisms of resistance in EGFRvIII expressing cells. For example, a dynamic downregulation of EGFRvIII in response to prolonged erlotinib treatment has been reported, as has derepression of PDGFRβ signaling, activation of a Urokinase-Bim signaling axis, and a glucose metabolism linked p53 pathway. However, not much is known about the early adaptive response that is triggered when the EGFR is inhibited in GBM. It has been reported that EGFR inhibition in GBM or lung cancer triggers a TNF-driven survival pathway that protects cancer cells from cell death resulting from EGFR inhibition. This work was repeated by another group with similar results. However, new preliminary data from an unbiased RNA-seq approach indicates that the response to EGFR inhibition is multifaceted and that a large component of the EGFR inhibition triggered adaptive response is TNF-independent (FIG. 3). This is consistent with previous reports suggesting that the adaptive response to targeted inhibition of RTK signaling pathways is broad and involve multiple signals. For example, a study of RAF inhibition using vemurafenib in melanoma revealed activation of at least 6 signaling pathways, with the JNK signaling pathway playing a key role in the adaptive response. Similarly, although EGFR inhibition in GBM leads to altered expression of a large number of genes as discussed below, targeted inhibition of one or a small number of pathways may cripple the adaptive response. Here, it is proposed to study the Nrf2 signaling pathway that is activated in response to EGFR inhibition in GBMs expressing either EGFRwt or EGFRvIII and is likely to be a key component of the resistance to EGFR inhibition in GBM. The rationale for prioritizing Nrf2 and specific downstream targets such as xCT-SLC7A11 is discussed in the preliminary data section. Since the EGFR is expressed in a majority of GBMs, EGFR inhibition could be an effective treatment for GBM if the accompanying Nrf2 mediated adaptive response is blunted.

GBM heterogeneity is a particular concern for treatments that target EGFRvIII, since expression of EGFRvIII may be focal in GBM. However, EGFRwt is expressed diffusely in the majority of tumor cells within a GBM. Also, a number of studies have shown that EGFRvIII is usually co-expressed with EGFRwt in GBM, and the two receptors activate each other. Thus, the treatment approach of combining EGFR+adaptive response inhibition is effective in the presence of EGFRwt and/or EGFRvIII and is not limited by the focal expression pattern/heterogeneity of EGFRvIII expression, and is effective in all EGFRwt and EGFRvIII expressing GBMs we have tested.

Therapeutic resistance to targeted inhibition remains a central problem in GBM. Identification of the Nrf2 signaling pathway as a key mediator of resistance to EGFR inhibition in GBM provides a critical new insight into mechanisms of therapeutic resistance in GBM. Because drugs are available that target Nrf2 or downstream signaling effectors, this could quickly lead to desperately needed new treatment for GBM patients. Furthermore, since the EGFR is widely expressed in GBM, a therapeutic approach combining inhibition of the EGFR and Nrf2 signaling pathways could be broadly useful in large subsets of GBM patients. Preliminary data indicate that activation of the Nrf2 signaling pathway is a universal response to EGFR inhibition in GBMs. It is important to note that the Nrf2 pathway drives multiple facets of gliomagenesis including cancer stem cells, resistance to conventional treatment, and a worse prognosis, suggesting that it could be a critically important pathway that mediates resistance to EGFR inhibition in GBM. Also, this study aims to discover biomarkers such as Nrf2 or downstream signals that may help to predict responsiveness to EGFR inhibition. Preliminary data indicate that a combined inhibition of EGFR and Nrf2 is highly effective in multiple preclinical orthotopic models of GBM (FIG. 6A-E).

2. Experimental Model and Preliminary Data

As an experimental model, GBMs from the widely used Mayo panel will be used. These PDX GBMs replicate features of the original GBM including invasiveness and are maintained as an NIH supported National PDX resource and obtained through collaboration with Dr. Jann Sarkaria. In this model, patient-derived xenografts are maintained by continuous passage in mice. The Mayo explant cultures are generated at Mayo Clinic from mouse tumors, shipped weekly, and generally used without passaging in the laboratory. Table 1 below shows a partial list of GBMs with molecular classification in the Mayo Xenograft panel.

TABLE 1

| Mayo | EGFR | Molecular |
|---|---|---|
| PDX | status | subtype |
| GBM6 | wt + vIII | C |
| GBM10 | Wt | M |
| GBM12 | Wt | M |
| GBM15 | Wt | NA |
| GBM22 | Wt | C |
| GBM26 | Wt | NA |
| GBM28 | Wt | NA |
| GBM36 | wt | P |
| GBM39 | wt + vIII | M |
| GBM44 | Wt | M |
| GBM46 | wt + vIII | M |
| GBM61 | Wt | P |
| GBM76 | wt + vIII | c |

Xenotransplant studies using human cells is the preferred model when testing a new therapeutic approach using drugs in mice. The advantages of using xenotransplants include high penetrance, short latency, and rapid uniform growth kinetics in vivo. While using immunocompromised mice imposes limitations, the nature of this study with its focus on identifying subsets of human GBMs that will respond to EGFR+Nrf2 inhibition requires the use of human samples. An immunocompetent model using murine glioma GL261 cells expressing EGFR has also been incorporated. See FIG. 6D-E.

Although the initial work focused on the role of a TNF-driven pathway in mediating resistance to EGFR inhibition in GBM, it is quite likely that additional pathways play a role. Thus, an unbiased investigation of the early adaptive response to EGFR inhibition in GBM was undertaken by conducting transcriptome analysis in explant cultures from Mayo PDX GBM12 (EGFRwt) after treatment with erlotinib. Without wishing to be bound by theory, these data indicate that EGFR inhibition leads to a broad transcriptional response. 3635 erlotinib-upregulated genes have been identified by RNAseq.

TABLE 2

| Erl-induced | | |
|---|---|---|
| Pathways | Genes | q-values |
| NRF2 pathway | 40 | 0.00124 |
| Validated targets of C-MYC | 23 | 0.0183 |
| transcriptional repression | | |
| Validated transcriptional | 19 | 0.0183 |
| targets of Tap63 isoforms | | |
| Axon guidance | 42 | 0.0221 |
| Ectoderm Differentiation | 35 | 0.0299 |
| Glutathione conjugation | 14 | 0.0375 |

Referring to Table 2, pathway analysis was performed on unregulated genes revealing six pathways to be enriched in these erlotinib-induced genes, with q-values less than 0.05. These q-values are corrected from p-values of the hypergeometric test, for multiple testing using the false discovery rate (FDR) method.

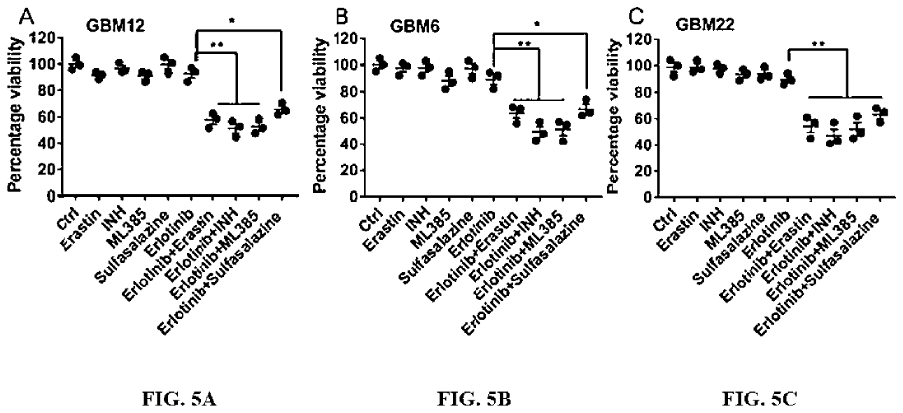

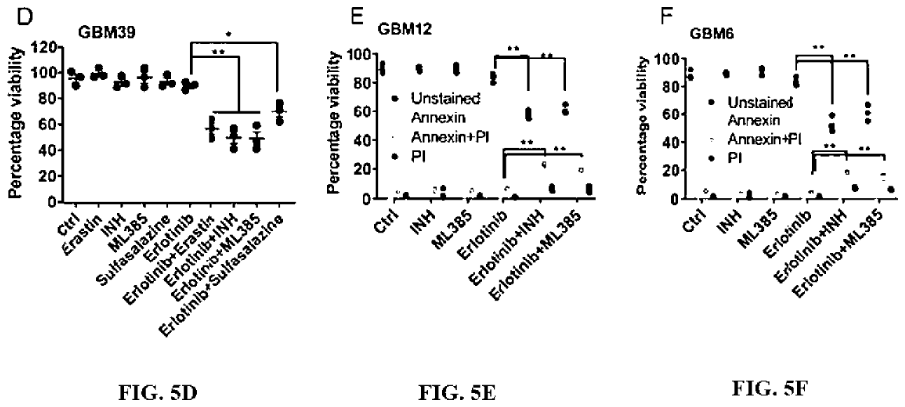

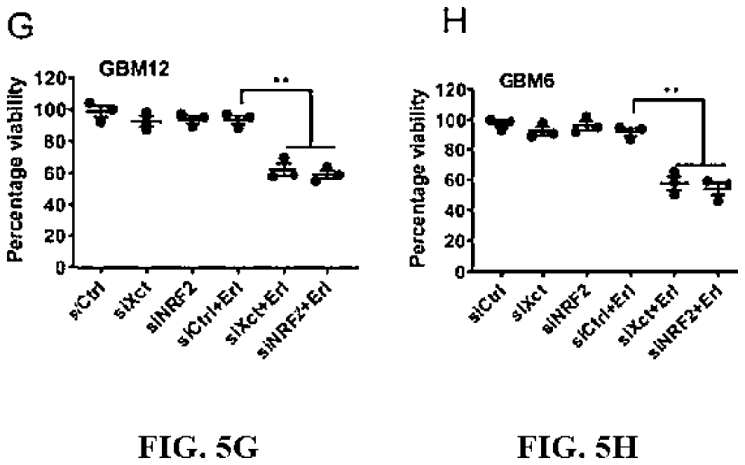

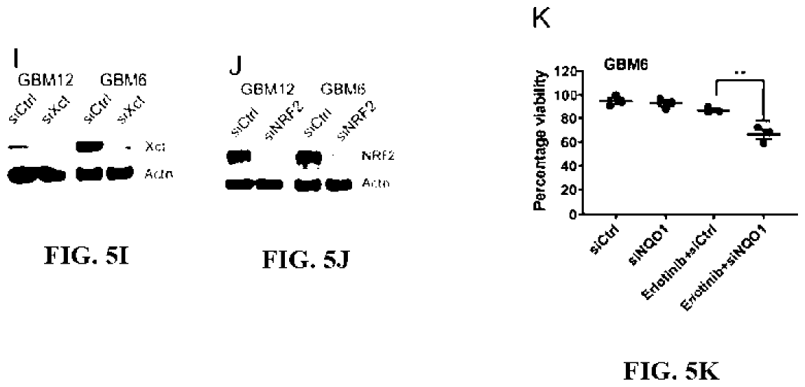

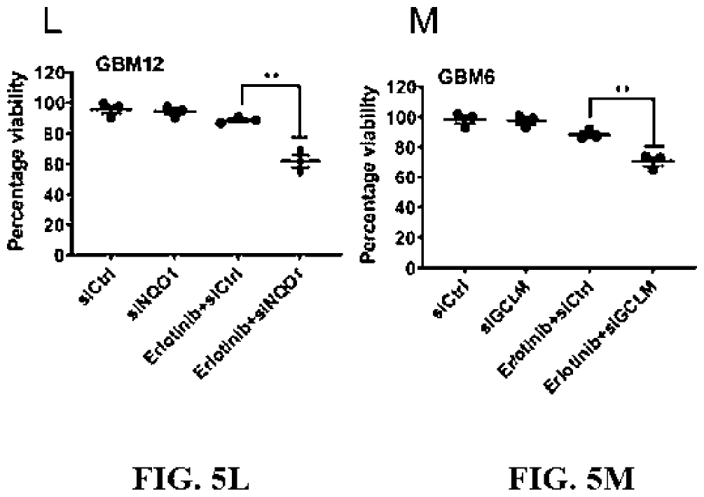

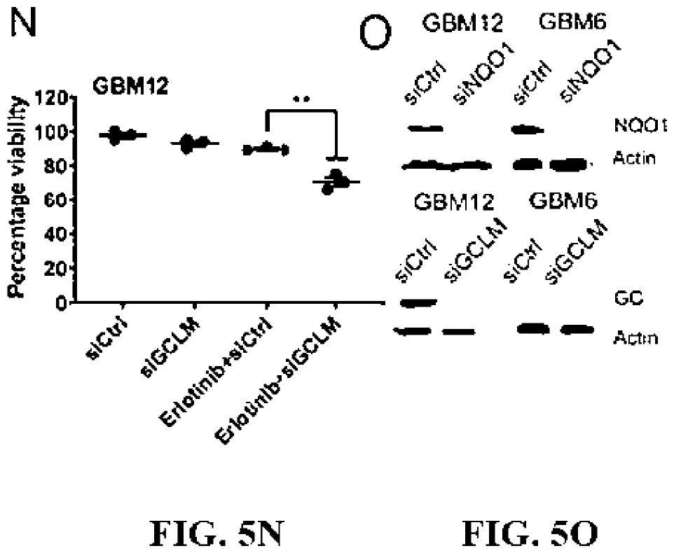

The Nrf2 network was prioritized based on the following criteria: (1) Statistical Significance and broad activation: Pathway analysis of 3635 genes revealed upregulation of 40 Nrf2 pathway/target genes with a q value of 0.00124 (Table 2 and FIG. 3). (2) Nrf2 drives multiple hallmarks of cancer including a role in gliomagenesis. NRF2 mutations occur in multiple cancers and confer constitutive activation and a worse prognosis. Keap-1, the major inhibitor of Nrf2 is frequently mutated in cancer, leading to increased Nrf2 activity and a worse prognosis. Nrf2 function in GBM is relatively unexplored. Although Nrf2/KEAP1 mutations are not common in glioma, upregulation of Nrf2 target genes was detected in 13.7% of anaplastic gliomas and 32.7% of GBM, suggesting a role in tumor progression. Nrf2 has a role in maintenance of glioma stem cells and in glioma cell survival and invasion. The Nrf2 target gene xCT-SLC7A11 promotes the malignant phenotype and seizures in GBM. (3) Relevance to therapeutic resistance: Nrf2 activation induces therapeutic resistance to chemotherapy, radiation and targeted treatment. (4) Independent of TNF signaling: Importantly, Nrf2 activation in response to EGFR inhibition is independent of the previously reported TNF-driven adaptive response and Nrf2 target genes cannot be blocked by TNF inhibition (FIG. 3). (5) Nrf2 activation in response to EGFR inhibition in GBM is a novel finding, and strong data showing that Nrf2 plays a key role in mediating therapeutic resistance to EGFR inhibition in preclinical models of GBM is disclosed herein (FIG. 5A-O and FIG. 6A-E).

3. Activation of the Nrf2 Signaling Network in Response to EGFR Inhibition.

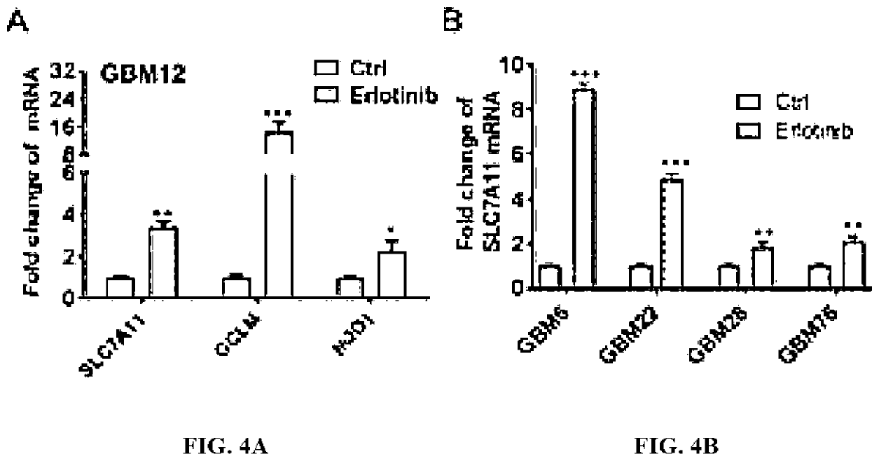

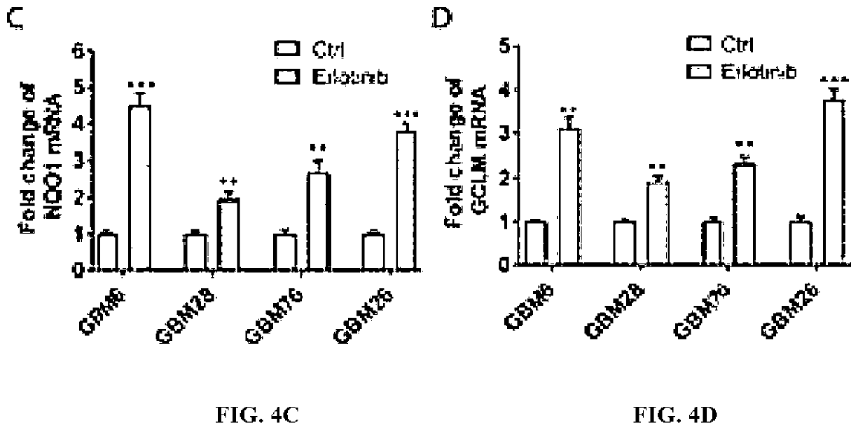

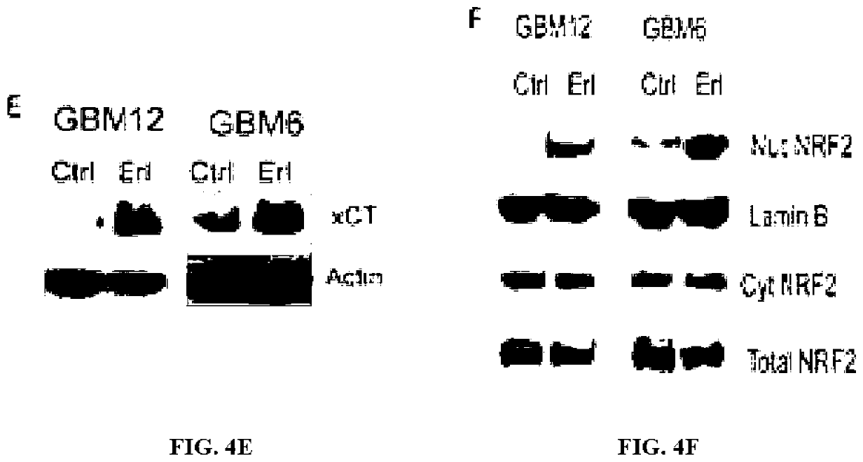

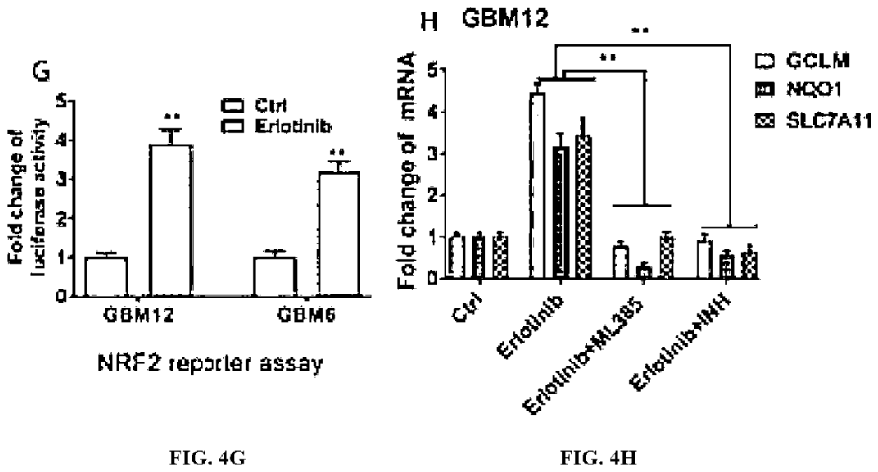

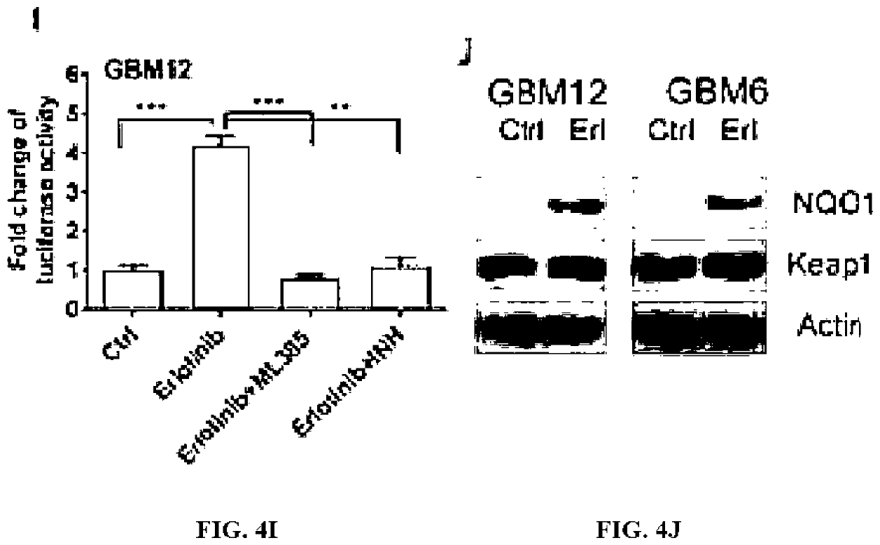

Nrf2 (nuclear factor erythroid 2-related factor 2) is a transcription factor that plays a central role in the cellular antioxidant response. There is substantial evidence that ROS facilitate malignant progression and resistance to therapy. It was found that Nrf2 regulated genes are unregulated in multiple Mayo PDX explant cultures in response to EGFR inhibition (FIG. 4A-E and FIG. 4J). While there is no increase in Nrf2 levels in response to EGFR inhibition, there is increased nuclear localization of Nrf2 (FIG. 4F) and increased transcriptional activity of an Nrf2 reporter (FIG. 4G). It was also confirmed that the erlotinib induced upregulation of putative Nrf2 target genes and increased Nrf2 transcriptional activity could be blocked by Nrf2 inhibitors, INH, and ML-385 (FIG. 4H-I).

Importantly, chemical or biological inhibition of Nrf2 or downstream genes synergized with EGFR inhibition to suppress glioma cell viability (FIG. 5A-0). However, Nrf2 inhibition alone does not affect viability of glioma cells. The preliminary data thus strongly support Nrf2 activation as a major component of the adaptive response to EGFR inhibition in GBM. The role of the Nrf2 target gene xCT/SLC7A11, NQO1, and GCLM were also examined and a significant synergistic effect with EGFR inhibition was found (FIG. 5A-O).

Referring to FIG. 5A, selective inhibition of Nrf2 or downstream targets sensitizes GBM12 cells to erlotinib. Cells were exposed to erlotinib with or without other inhibitors followed by Alamarblue cell viability assay after 72 hours. ML385 (1 μm) and isoniazid (INH 1 μm) are Nrf2 inhibitors while Erastin and sulfasalazine (5 μm) are xCT inhibitors. Erl. vs. Erl.+Erastin: P=0.0070; Erl. vs Erl.+INH.: P=0.0045; Erl. vs. Erl.+ML358: P=0.0038; Erl. vs Erl.+Sulf.: P=0.014.

Referring to FIG. 5B-D, similar experiments were conducted in GBM6, GBM22, and GBM39 cells. GBM6: Erl. vs. Erl.+Erastin: P=0.0089; Erl. vs Erl.+INH: P=0.0054; Erl. vs. Erl.+ML358: P=0.0049; Erl. vs. Erl.+Sulf.: P=0.026; GBM22: Erl. vs. Erl.+Erastin: P=0.0076; Erl. vs Erl.+INH: P=0.0060; Erl. vs. Erl.+ML358: P=0.0087; Erl. vs Erl.+Sulf.: P=0.0090; GBM39: Erl. vs. Erl.+Erastin: P=0.0083; Erl. vs. Erl.+INH: P=0.0050; Erl. vs. Erl.+ML358: P=0.0064; Erl. vs. Erl.+Sulf.: P=0.018.

Referring to FIG. 5E, GBM12 cells were indicated with drugs for 72 hours followed by Annexin-FACS assay. Unstained cells represent viable cells. Annexin positive cells are undergoing apoptosis. PI (Propidium iodide) positive cells are undergoing late apoptosis. Unstained: Erl. vs Erl.+INH: P=0.0047; Erl. vs. Erl.+ML358: P=0.0060; Annexin+PI: Erl. vs Erl.+INH: P=0.0022; Erl. vs. Erl.+ML358: P=0.0039.

Referring to FIG. 5F, a similar experiment was performed in GBM6. Unstained: Erl. vs Erl.+INH: P=0.0077; Erl. vs. Erl.+ML358: P=0.0093; Annexin+PI: Erl. vs Erl.+INH: P=0.0030; Erl. vs. Erl.+ML358: P=0.0042.

Referring to FIG. 5G and FIG. 5H, Nrf2 or xCT knockdown sensitize cells to erlotinib treatment. GBM12: siCtrl+Erl. vs. siNRF2+Erl.: P=0.0082; siCtrl+Erl. vs. siXct+Erl.: P=0.0059; GBM6: siCtrl+Erl. vs. siNRF2+Erl.: P=0.0087; siCtrl+Erl. vs. siXct+Erl.: P=0.0070. Data are presented as mean±SEM of at least 3 independent experiments. *P<0.05, ** P<0.01.

Referring to FIG. 5I and FIG. 5J, Western blots showing silencing of Nrf2 or Xct are shown.

Referring to FIG. 5K and FIG. 5L, NQO1 knockdown sensitize cells to erlotinib treatment. GBM12: siCtrl+Erl. vs. siNQOi+Erl.: P=0.0030. GBM6: siCtrl+Erl. vs. siNQO1+Erl.: P=0.0075.

Referring to FIG. 5M and FIG. 5N, GCLM knockdown sensitize cells to erlotinib treatment. GBM12: siCtrl+Erl. vs. siGCLM+Erl.: P=0.0020. GBM6: siCtrl+Erl. vs. siGCLM+Erl.: P=0.0065.

Referring to FIG. 5O, Western blots showing silencing of NQO1 and GCLM are shown.

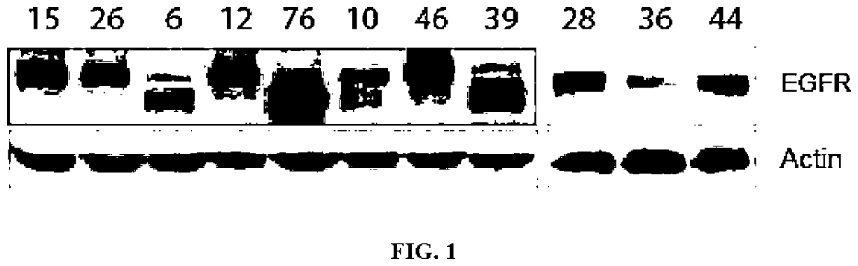

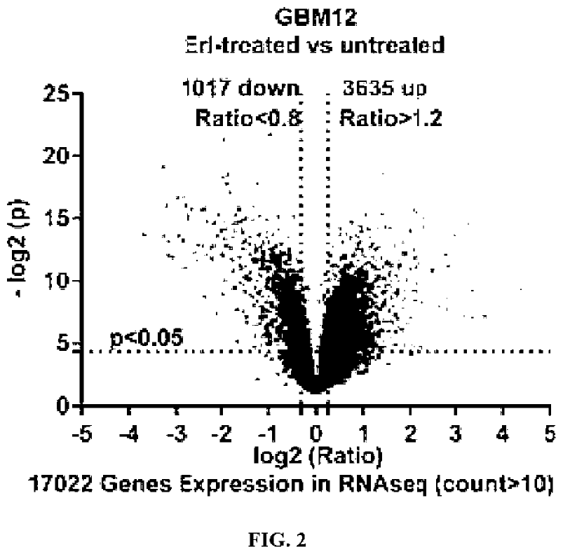

Importantly, the cell survival assay demonstrating that (A) the GBM lines studied are all resistant to EGFR alone and (B) this resistance can be overcome with a Nrf2 pathway inhibition are also validated in preliminary animal experiments. Data with two Mayo PDX lines and also an immunocompetent model is shown. GBM6 is a Mayo PDX line that expresses EGFRvIII while GBM12 expresses EGFRwt (FIG. 1 and Table 1). Temozolomide (TMZ) is the standard of care in the treatment of GBM, even though its effect on overall survival remains modest. Previous studies have shown that TMZ is most effective in the 45% of GBMs in which MGMT expression is suppressed (Hegi, et al. (2005) MGMT gene silencing and benefit from temozolomide in glioblastoma, N Engl J Med 352, 997-1003). Also, TMZ is, by far, the most effective drug in animal models of GBM. In FIG. 6A, it is shown that for GBM6, a GBM PDX with unmethylated MGMT, TMZ is ineffective while erlotinib plus INH is highly effective in preventing tumor growth.

4. Elucidation of the Mechanisms Downstream of Nrf2 Activation that Mediate Resistance to EGFR Inhibition.

Here, the role of Nrf2 activated signaling networks in mediating resistance to EGFR inhibition in GBM is examined.

GSH. EGFR inhibition induced Nrf2 activation results in increased expression of key proteins involved in glutathione (GSH) biosynthesis (FIG. 3). These include glutamate-cysteine ligase catalytic subunit (GCLC) and glutamate-cysteine ligase modifier subunit (GCLM) and GSR (Glutathione Reductase). GSR (Glutathione Reductase) is also upregulated in response to EGFR inhibition and is a key enzyme involved in GSH maintenance. GSH is a tripeptide composed of glutamate, cysteine and glycine. GSH is an important antioxidant that attenuates oxidative damage induced by multiple ROS species. Multiple studies have shown that GSH may play an important role in resistance to chemotherapy and targeted treatment in cancer including GBM.

Determination of GSH levels will be undertaken at a Biochemistry Core Facility at UT Southwestern using metabolite profiling as previously described (Krall, et al. (2017) KEAP1 loss modulates sensitivity to kinase targeted therapy in lung cancer, Elife 6; Yuan, et al. (2012) Nat Protoc 7, 872-881). Samples will be analyzed by liquid chromatography tandem mass spectrometry (LC-MS/MS) using positive ion/negative ion polarity switching via selected reaction monitoring (SRM) on a 5500 QTRAP hybrid triple quadrupole mass spectrometer. Peaks will be integrated using MultiQuant 2.1 data analysis software (AB/SCIEX). The ratio of reduced intracellular glutathione to oxidized glutathione is a measure of oxidative stress. A preliminary metabolite profiling experiment is shown in Table 3, demonstrating that the ratio of reduced to oxidized glutathione is increased in erlotinib induced cells, indicating that EGFR inhibition leads to increased oxidative stress.

TABLE 3

| Treatment | GSH/GSSG |
|---|---|
| Vehicle | 31.9 ± 7.7 |
| Erlotinib | 68.5 ± 15.2 |

Referring to Table 3, the GSH/GSSG ratio is significantly increased in erlotinib compared to vehicle treated cells. The experiment was conducted in Mayo PDX GBM12 cells after a 24 hour exposure with erlotinib or vehicle control. Data are presented as means±SD of four experiments. P<0.05. GSH, reduced glutathione; GSSG, oxidized glutathione. This result was also confirmed by a second experiment using a using GSH/GSSG-Glo Glutathione Assay (Promega, Madison Wis.) following the manufacturer's instructions.

TABLE 4

| Cell lines | Ctrl | Erlotinib |
|---|---|---|
| GBM12 | 23.7 ± 3.4 | 50.6 ± 6.6** |
| GBM6 | 12.1 ± 1.6 | 32.4 ± 4.2** |

**P < 0.01

Referring to Table 4, GSH/GSSG ratio in PDX cells treated with erlotinib is shown. GBM 12 and GBM6 cells were treated with 1 μM erlotinib for 24 h. Cells were lysed and analyzed with the Promega kit. Both GBM12 and GBM6 cells showed a statistically significantly increase in GSH/GSSG ratios comparing to control (DMSO) treated cells. Data are presented as means±SD of three experiments.

Biological role of GSH. First, it will be confirmed that Nrf2 activation is essential for the erlotinib induced increase in GSH/GSSG ratio by chemical or biological inhibition of Nrf2. Then, the biological function of GSH in mediating resistance to EGFR inhibition will be examined by a loss of function approach by using siRNA knockdown of GCLC, GCLM GSR, and cell viability assays using methods described previously (Guo, et al. (2017) A TNF-JNK-Axl-ERK signaling axis mediates primary resistance to EGFR inhibition in glioblastoma, Nat Neurosci 20, 1074-1084; Gong, et al. (2018) TNF-driven adaptive response mediates resistance to EGFR inhibition in lung cancer, J Clin Invest 128, 2500-2518). In addition, APR-246, a drug that that binds to GSH and depletes its intracellular levels, will be used. APR-246 synergizes with chemotherapeutic drugs, is effective in PDX models, and is currently being tested in multiple clinical trials. If cell viability assays are promising, animal studies will be undertaken.

GPX3 (Glutathione Peroxidase). GPX3 is upregulated in GBM in response to erlotinib (FIG. 3). GPX3 is involved in ROS homeostasis, acts in cooperation with GSR and has been implicated in the progression of cancer. Upregulation of GPX3 will be confirmed, followed by examination of the biological role of GPX3 by using siRNA knockdown or chemical inhibition using Tiopronin, which inhibits multiple GPX isoforms.

Multidrug resistance associated proteins and Drug metabolizing enzymes. These proteins play an important role in therapeutic resistance. The ABC transporters are responsible for the movement of substrates through the cells membrane, while drug metabolizing enzymes help in the detoxification of substrates including drugs. Drug metabolizing enzymes include glutathione S-transferases.

Multi drug resistance associated proteins. The adenosine triphosphate (ATP)-binding cassette (ABC) superfamily efflux both cytotoxic agents and targeted anticancer drugs using ATP driven energy resulting in resistance to treatment. ABC transporters are expressed in cancer including glioma and may correlate with worse prognosis. Without wishing to be bound by theory, the data indicate that ABCC2, ABCC3, and ABCC5 are upregulated in response to EGFR inhibition in glioma cells (FIG. 3). ABCC2 (MRP2) substrates comprise a broad range of drugs that include chemotherapy drugs. Thus, ABC transporters could mediate resistance to EGFR inhibition in GBM by increasing efflux of erlotinib out of tumor cells and possibly out of the blood brain barrier. The upregulation of ABC transporter proteins will be confirmed by Western blot. The effect of siRNA knockdown of ABCC2, ABCC3, and ABCC5, alone or in combination in erlotinib treated cells, will be examined using cell viability assays. If cell viability assays are promising, whether ABC transporters affect cellular/CNS levels of erlotinib in glioma cells will be examined using methods discussed herein.

Glutathione S-transferases isoenzymes. Glutathione S-transferase isoenzymes detoxify drugs and are phase II detoxifying enzymes protecting from oxidative stress and promoting therapeutic resistance. Multiple GST isoenzymes are upregulated in response to EGFR inhibition (including GSTP1, GSTM2, GSTM3, GSTM4, and GSTM5). GSTs function via their catalytic activities that include the conjugation of byproducts of oxidative stress and xenobiotics to glutathione (GSH), and, thus, protect cells. The effect of siRNA knockdown of GSTP1, GSTM2, GSTM3, GSTM4 or GSTM5, alone or in combination, will be examined in erlotinib treated cells using cell viability assays and animal experiments.

xCT/SLC7A11. The NRF2 target gene xCT/SLC7A11 promotes the malignant phenotype and seizures in GBM. xCT is a membrane transporter that couples the influx of extracellular cysteine to the efflux of glutamate. Preliminary data for xCT/SLC7A11 are presented in FIG. 4A, FIG. 4B, and FIG. 4E. xCT/SLC7A11 experiments were prioritized and it was shown that inhibition of xCT/SLC7A11 using chemical inhibitors sulfasalazine or Erastin synergizes with EGFR inhibition in cell survival assays (FIG. 5A and FIG. 5B). A similar result was obtained with siRNA knockdown of xCT/SLC7A11 (FIG. 5G-I).

NQO1. NAD(P)H: quinone acceptor oxidoreductase 1 (NQO1) is a multifunctional antioxidant enzyme, regulated by Nrf2 and is cytoprotective. Preliminary data indicates that NQO1 is upregulated by EGFR inhibitors (FIG. 3, FIG. 4A-J, and FIG. 5A-O). Thus, the role of NQO1 will be examined using a loss of function approach with siRNA knockdown and cell survival assays and animal experiments.

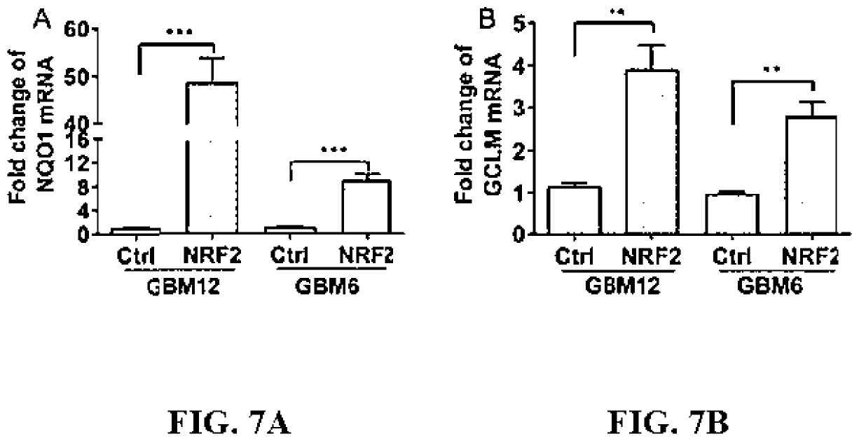

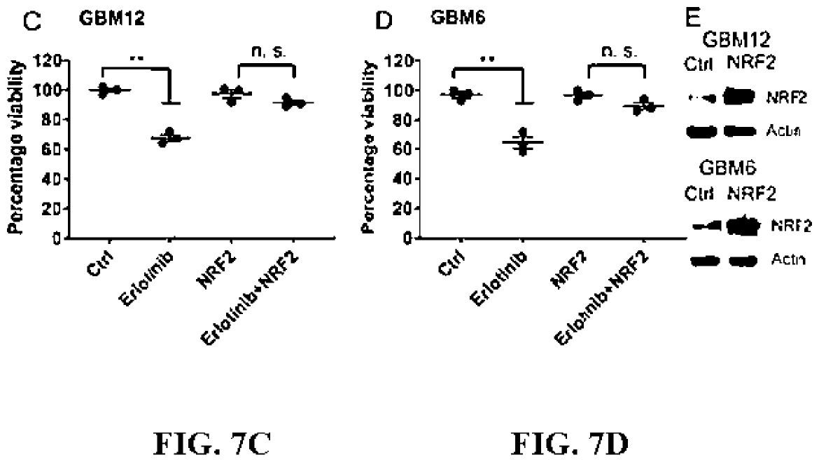

Direct modulation of Nrf2. Direct modulation will be undertaken with the above experiments to confirm the role of Nrf2 in activation of these pathways by undertaking siRNA knockdown of Nrf2 (as shown in FIG. 5A-O) and also overexpression of Nrf2 using a wild type Nrf2 (FIG. 7A-D), or a Keap-1 resistant constitutively active mutant (delNEH2).

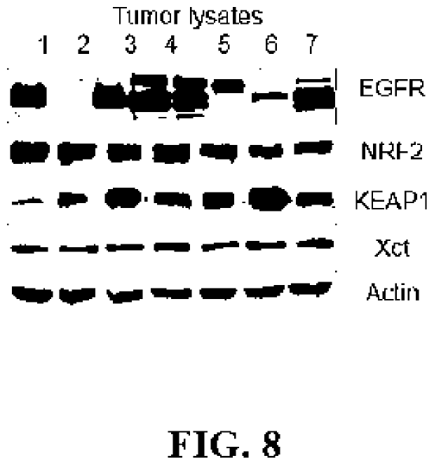

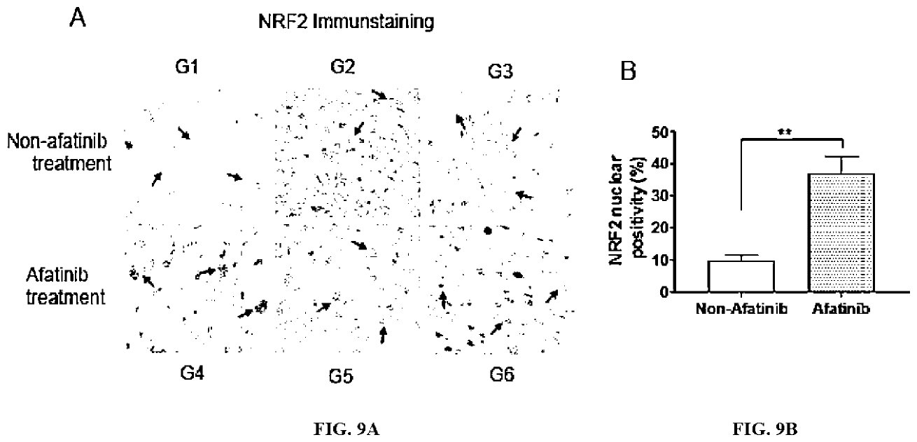

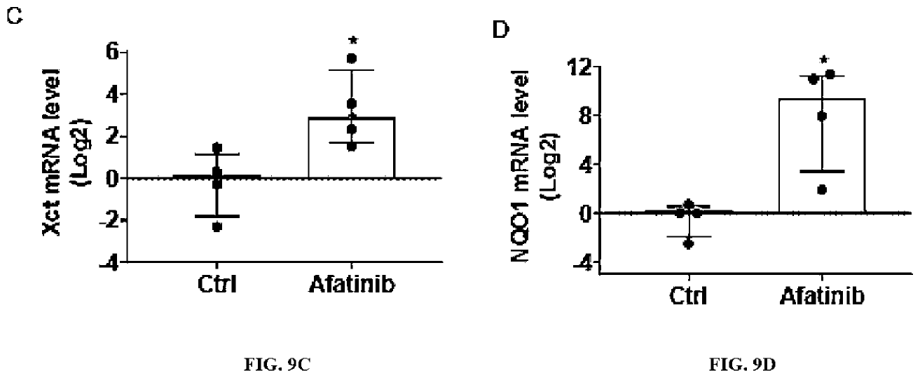

Analysis in tumor tissue. A preliminary analysis in seven resected GBM samples indicates that key Nrf2 components are broadly expressed in GBMs (FIG. 8). Resected tumors were frozen at −80 degrees followed by extraction of protein and Western blot.

Analysis in post EGFR TKIs treated GBMs. Post treatment GBM tissue was obtained from 4 patients (provided by Dr. Kesari) and compared to TKI naive GBM patient tissue. Whether treatment with EGFR TKI leads to activation of Nrf2 in human GBM was examined by studying nuclear localization of Nrf2 and expression of Nrf2 target genes. It was found that Nrf2 is indeed activated in GBM tissue from EGFR TKI treated, and could be a biomarker to predict treatment responses or prognosis in GBM.

It was found that the biological effects of EGFR inhibition using erlotinib and afatinib (first and second generation EGFR TKIs) in GBM models are quite similar. Dr. Kesari has indicated that based on the enrollment in his afatinib clinical trial, he expects to be able to provide post-afatinib treatment tissue from at least 10 more patients. The samples provided by Dr. Kesari will be relabeled to a random, non-linked code.

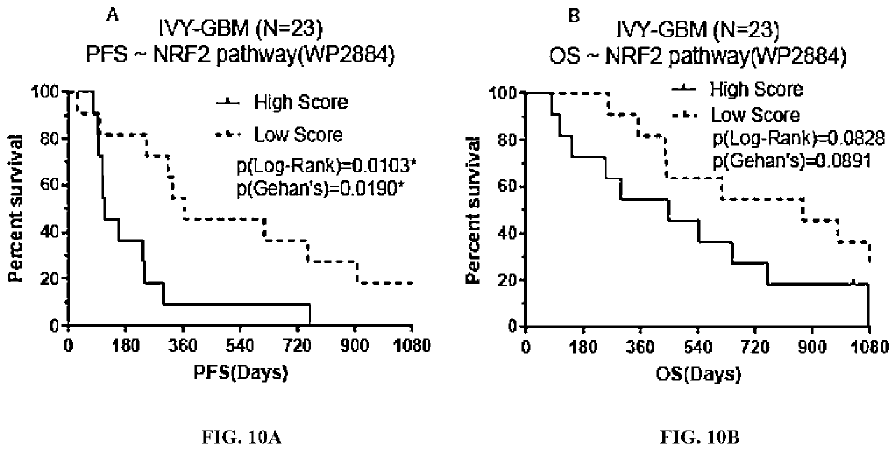

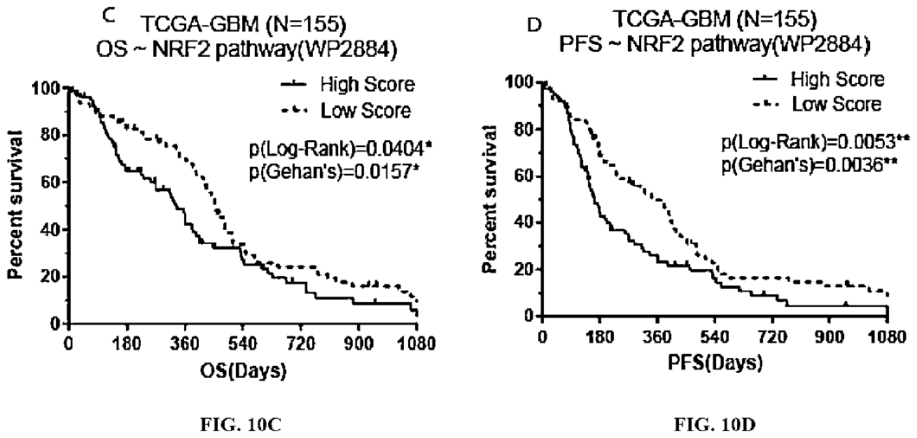

Next, whether an Nrf2 target gene signature predicts prognosis in patients with GBM was examined. First, the Ivy GBM data was evaluated. The Ivy Glioblastoma Atlas Project collected 23 GBM patient's data. KM-plot shows patients with higher NRF2 scores have significant shorter PFS, with p-values=0.01-0.02 in log-rank and Gehan's test. Thus, a high level of Nrf2 target genes confers a statistically significant adverse effect on the PFS in this dataset (FIG. 10A). However, effect on the overall survival was not significant (FIG. 10B). Then, an analysis of the much larger TCGA GBM collection was undertaken, which demonstrates that a higher level of Nrf2 target genes has a significant adverse effect on overall and progression free survival (FIG. 10C and FIG. 10D.

Referring to FIG. 10A and FIG. 10B, IVY-GBM PFS/OS are shown. The Ivy Glioblastoma Atlas Project performed RNAseq on 23 GBM patients' bulk tumors and collected their survival data. For individual cases, all 62474 gene expression profiles were scored with the signature of NRF2 pathway (wikipathway: WP2884, 142 genes), using the R package ssGSEA2.0 (single sample Gene Set Enrichment Analysis). Higher scores represent patients with more overall-activated NRF2 pathway. Kaplan-Meier survival analysis was conducted between patients with high and low 50% scores. The log-rank and Gehan's tests were used to calculate the significance, using Graphpad7.0. C-D. The TCGA database collected 155 GBM patients' tumor RNAseq data and associated survival data. The single sample Gene Set Enrichment Analysis (ssGSEA) by R was performed on these 155 cases' total 60483 gene expression profiles, and then divided by the high and low 50% of signature scores of NRF2 pathway (wikipathway: WP2884, containing 142 genes). KM-plot shows patients with higher NRF2 scores will have significant shorter PFS (p<0.01) and OS (p<0.05) in the log-rank and the Gehan's tests. An analysis of key Nrf2 target genes (for example xCT/SLC7A11, GCLC, GCLM) will be undertaken using data from the IVY GBM and TCGA data to determine the effect on prognosis. Studies on the effects of biomarkers such as RIP1 and TRADD on prognosis in GBM have been previously published (Chakraborty, et al. (2013) Cytoplasmic TRADD Confers a Worse Prognosis in Glioblastoma, Neoplasia 15, 888-897; Park, et al. (2009) The receptor interacting protein 1 inhibits p53 induction through NF-kappaB activation and confers a worse prognosis in glioblastoma, Cancer Res 69, 2809-2816).

Although Nrf2 can be targeted directly in combination with the EGFR, it may be advantageous to target multiple components of the signaling pathway. Without wishing to be bound by theory, these studies will help to identify additional targets such as GCLM (FIG. 5A-O) that will be tested in an animal model. Nrf2 induces additional target genes that are not prioritized in the initial experiments. For example, a group of Nrf2 induced transporter proteins (FIG. 3) that includes SLC6A2 (sodium, norepinephrine), SLC2A11, SLC2A12 (transport of sugars) SLC6A16 (Na and Cl transport), SLC5A12, sodium coupled transporter SLC6A9 (sodium and chloride dependent glycine transporter), and a group of genes related to heme metabolism (FTH1, ferritin heavy chain 1, BLVRD, biliverdin reductase B and FTL, ferritin light chain, FIG. 3) are upregulated in response to EGFR inhibition and could be examined as an alternative strategy or in future experiments. In addition, non-Nrf2 targets will also be considered if Nrf2 fails to validate as a critical mediator of EGFR resistance. An example would be induction of Myc target genes (Table 2).

5. Elucidation of the Mechanism of EGFR TKI-Induced Nrf2 Activation.

Nrf2 is negatively regulated by Kelch-like ECG associated protein 1 (KEAP1), an adaptor protein linked to the CUL3 E3 ubiquitin ligase that targets Nrf2 for proteasomal degradation. Oxidative stress leads to the oxidation of Keap1 at key cysteine residues causing a conformational change in Keap1 leading to release of Nrf2 and nuclear translocation where it binds to ARE (antioxidant responsive element) sequences to activate transcription of target genes. Activation of Nrf2 can also occur through Keap1 independent or non-canonical mechanisms. The experiments will be undertaken in explant cultures of multiple Mayo PDX lines using exposure to erlotinib for various time points (ranging from 15 minutes to 24 h). Similar experiments will be undertaken in tumor tissue from intracranial tumors (detected by BLI) in mice treated with erlotinib for 12 h, 24 h, 48 h and 72 h.

Oxidative stress. Without wishing to be bound by theory, it is hypothesized that a sudden inhibition of EGFR signaling may increase oxidative stress and thus activate Nrf2. Previous studies have reported that EGFR inhibition leads to increased oxidative stress in lung and head and neck cancers (Orcutt, et al. (2011) Erlotinib-mediated inhibition of EGFR signaling induces metabolic oxidative stress through NOX4, Cancer Res 71, 3932-3940; Qian, et al. (2009) Clin Exp Pharmacol Physiol 36, 487-494). Here, whether EGFR inhibition results in increased oxidative stress and ROS production, and whether blocking ROS production results in inhibition of erlotinib induced Nrf2 activation will be examined. MitoTracker® Red CM-H2XRos (Fisher), a reduced nonfluorescent dye which is taken up passively by living cells, will be used. In the mitochondria, the dye is oxidized by superoxide, resulting in the emission of red fluorescence.

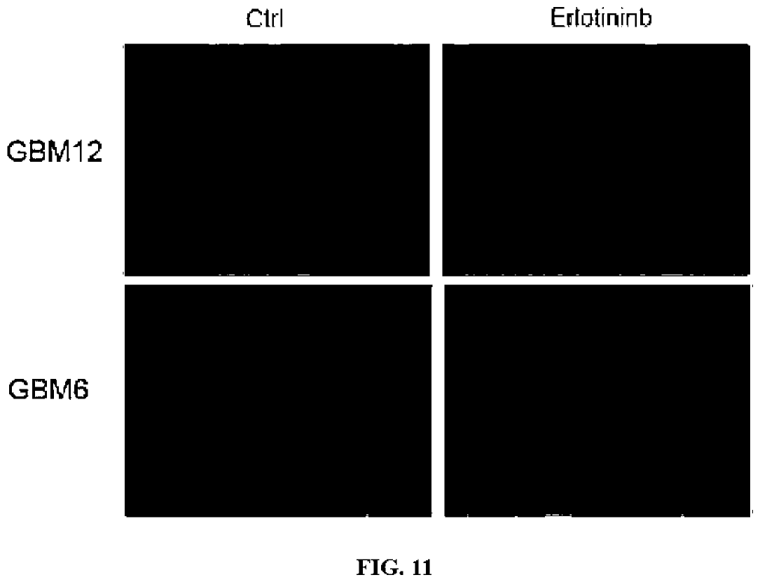

Referring to FIG. 11, GBM 12 or GBM6 cells were treated with erlotinib (1 uM) or vehicle for 24 hours. MitoTracker® Red CM-H2XRos (Fisher) was added to culture media at a concentration of 100 nM and incubated at 37° C. in a humidified incubator containing 5% CO₂ for 30 minutes, the cells were then fixed in ice-cold methanol for 15 min at −20° C. followed by fluorescence microscopy.

These initial experiments demonstrate that there is an increase in ROS following EGFR inhibition in glioma cells using Mitotracker dye (FIG. 11). Additionally, flow cytometry using a fluorescent dye based free radical sensor Carboxy-H2DCFDA will be used. ROS scavengers such as N-acetyl-L-cysteine (NAC), 4,5-dihydroxybenzene-1,3-disulfonate (Tiron), and catalase will be used to pretreat GBM cells before erlotinib exposure. Then, the intracellular ROS levels will be monitored by flow cytometry, and Nrf2 activity will be assessed by nuclear localization, reporter activity and transcription of downstream genes. ROS will be detected in animal tumors by using immunohistochemistry with antibody to 4HNE (4-hydroxy-2-noneal), which detects lipid peroxidation.

Canonical Pathway. Keap1 is the protein primarily responsible for regulation of Nrf2. Keap1 binds to Nrf2 and sequesters it in the cytosol rendering it inactive. When the cell encounters an oxidative change, multiple cysteine residues in Keap1 become oxidized and release Nrf2. Preliminary data indicate that the cellular level of total Keap1 is not changed by erlotinib (data not shown). The oxidation of Keap1 in GBM cells and animal tumors exposed to erlotinib will be examined as described previously (Fourquet, et al. (2010) Activation of NRF2 by nitrosative agents and H₂O₂ involves KEAP1 disulfide formation, J Biol Chem 285, 8463-8471). Briefly, Keap1 oxidation will be tested using protein electrophoresis to detect altered mobility in non-reduced samples.

Non-canonical pathways. Nrf2 can also be activated by a number of non-canonical pathways that will be explored as outlined below.

p62. p62/SQSTM1, a scaffold protein, is known to activate Nrf2 and may play a role in gliomagenesis. p62 interacts with Keap1 and induces an autophagy dependent degradation of Keap1 with resultant stabilization and activation of Nrf2. A loss of function approach will be used to examine the role of p62 in EGFR inhibition induced activation of Nrf2.

Dipeptidyl peptidase III (DPP3). DPP3 is a zinc aminopeptidase that also participates in the regulation of oxidative stress by decreasing the Nrf2-Keap1 interaction and increasing activation of Nrf2. DPP3 is overexpressed in breast cancer, correlates with activation of Nrf2 and confers a worse prognosis.

BRCA1. Breast cancer type 1 susceptibility protein (BRCA1) a tumor suppressor that protects cells against oxidative stress by increasing Nrf2 transcriptional activity. BRCA1 interacts with Nrf2 and prevents Keap1 dependent ubiquitination of Nrf2, and also regulates Nrf2 at a transcriptional level. Although, BRCA1 is a known tumor suppressor, it may have an oncogenic role in glioblastoma. A loss of function approach will be used to examine the role of BRCA1 in EGFR inhibition induced activation of Nrf2 as outlined above.

Other proteins. Other proteins that have been implicated in non-canonical activation of Nrf2 include WTX encoded by the Wilm's tumor gene that binds to Keap-1 to prevent the Nrf2-Keap1 interaction. Prothymosin α is a nuclear protein that interacts with Keap1 leading to increased Nrf2 activation. The protein partner and localizer of BRCA2 (PALB2) also interacts with Keap1 and activates Nrf2. The role of these proteins in GBM is unknown. A loss of function approach will be used to examine the role of these proteins in EGFR inhibition induced activation of Nrf2 as outlined above.

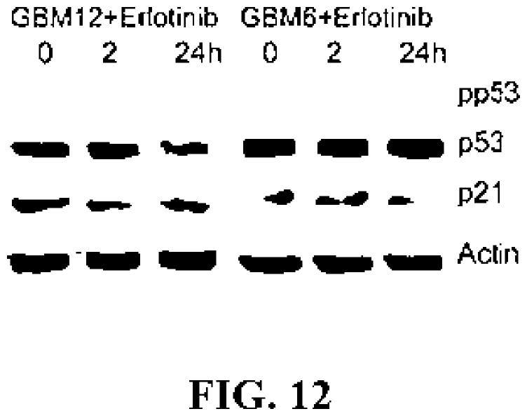

p21. p21Cip1/WAF1 is a cyclin-dependent kinase inhibitor that regulates the cell cycle and acts as an antioxidant. The antioxidant function of p21 is mediated through stabilization of Nrf2. Thus, p21 is an interesting target for study. However, initial data indicate that p21 is not upregulated in response to erlotinib (FIG. 12). Similarly, p53 is neither induced nor phosphorylated in response to erlotinib (FIG. 12, right).

In these studies, the major goal is to identify the mechanisms that lead to Nrf2 activation when the EGFR is inhibited. Preliminary data indicate that EGFR inhibition leads to increased ROS. Thus, it is likely that EGFR inhibition leads to Nrf2 activation, at least in part, via the canonical pathway of Keap1 oxidation. However, it is possible that non-canonical mechanisms play a role in Nrf2 activation, and thus, these studies may uncover additional targets in the Nrf2 signaling network for optimal inhibition of the Nrf2 pathway in combination with EGFR inhibition. Off-target effects of shRNA will be addressed by re-expressing the gene using shRNA resistant cDNA generated by introducing one or more synonymous mutations to the region of the cDNA that is targeted by the shRNA.

TABLE 5

| | | | AUC, | Cmax, | | |
|---|---|---|---|---|---|---|
| Compound | Applied | Dose | ng * hr/ml | ng/mL | T½, hr | Ref. |
| Erlotinib | Human | 150 mg | 11,860 ± | 872 ± | n.a. | Raizer, et al. |
| | | QD | 5,010 | 399 | | (2010) Neuro |
| | | | | | | Oncol 12, 95- |
| | | | | | | 103. |
| | Mouse | 12.5 mg/kg | 27,042 ± | 3,513 ± | 3.1 | Sivanand, et al., |
| | | QD | 2,569 | 271 | | (2012) Sci |
| | | | | | | Transl Med 4, |
| | | | | | | 137ra175. |
| Afatinib | Human | 50 mg QD | 459-956 | 30-40 | 60.3 | Schnell, et al. |
| | | | (~1-2 | (60-80 | | (2014) Cancer |
| | | | μM * hr) | nM) | | Chemother |
| | | | | | | Pharmacol 74, |
| | | | | | | 267-275; Wind, |
| | | | | | | et al. (2017) Clin |
| | | | | | | Pharmacokinet |
| | | | | | | 56, 235-250. |
| | Mouse | 30 mg/kg | (2375 | (417 nM) | 5 | Zhang, et al. |
| | | QD | nM * hr) | | | (2017) Acta |
| | | | | | | Pharmacol Sin |
| | | | | | | 38, 233-240. |
| Erastin | Human | No data | | | | |
| (xCT) | Mouse | 10-30 | No PK data published | | | Huo, et al. |
| | | mg/kg QD | | | | (2016) PLoS |
| | | for 4 wks | | | | One 11, |
| | | | | | | e0154605. |
| Sulfazaline | Human | 250 mg | SASP: | SASP: | SASP: | Gu, et al. (2011) |
| | | | 48,500 | 5351 | 9.8 | Journal of |
| | | | (122 | SP: 829 | SP: 8.5 | chromatography. |
| | | | μM * hr) | 5-ASA: | 5-ASA: | B, Analytical |
| | | | SP: 7297 | 259 | 11 | technologies in |
| | | | (20 | | | the biomedical |
| | | | μM*hr) 5- | | | and life sciences |
| | | | ASA: | | | 879, 449-456. |
| | | | 3001 | | | |
| | Mouse | 67.5 mg/kg | SASP: 21 | | SASP: | Zheng, et al. |
| | | QD for 3 | μM * hr | | 0.9 | (1993) Drug |
| | | wks | SP:430 | | SP: 2.2 | Metab Dispos |
| | | | μM * hr | | | 21, 1091-1097. |
| | | | AcSP: 44 | | | |
| | | | μM * hr | | | |
| INH | Human | 150 mg | 19.7 mg * hr/l | 4.83 mg/l | n.a. | Bhatt, et al. |
| (Isoniazid) | | QD for 6 | | | | (2014), |
| | | months | | | | Antimicrob |
| | | | | | | Agents |
| | | | | | | Chemother 58, |
| | | | | | | 3182-3190. |
| | Mouse | 25 mg/kg | 27.3 mg-h/L | 22 mcg/ml | n.a. | Almeida, et al. |
| | | QD for 4 | | | | (2009) |
| | | wks | | | | Antimicrob |
| | | | | | | Agents |
| | | | | | | Chemother 53, |
| | | | | | | 4178-4184. |

| ML385 | Human | No data | |
|---|---|---|---|
| (Nrf2) | Mouse | 30 mg/kr | Singh, et al. |
| | | i.p. QD for | (2016) ACS |
| | | 4 wks | chemical biology |
| | | | 11, 3214-3225. |

6. Examination of the Biological Effect of a Combined Inhibition of EGFR and Nrf2 or Nrf2 Downstream Targets in a Preclinical Mouse Orthotopic Model.

Multiple Mayo PDX lines starting with GBM12 (EGFRwt) and GBM6 (EGFRvIII) expressing luciferase will be used for the initial set of experiments. An intracranial model will primarily be used, and subcutaneous tumors will be used in limited experiments where CNS penetration of a particular drug is uncertain. Preliminary data are shown in FIG. 5A-O and FIG. 6A-E.

A dosing regimen for mice that will mimic drug exposures observed in humans will be defined, and then the studies will be repeated using these conditions. Preliminary evaluation of published pharmacokinetics is presented in Table 5. PK/PD studies will be undertaken on all of these drugs and confirmed, before further refining the doses used in mice to match human and mouse exposures and to determine whether combination therapy affects the pharmacokinetics of any of the drugs. To align these studies with the post-afatinib clinical samples provided by Dr. Kesari, and as an additional validation, afatinib will be used in animal experiments (http://www.bccancer.bc.ca/drug-database-site/Drug %20Index/Afatinib monograph.pdf).

Subaim 3A. Next, the role of Nrf2 in mediating primary resistance to erlotinib will be examined in an orthotopic xenotransplant and an immunocompetent model. Here, whether inhibition of Nrf2 will confer sensitivity to EGFR inhibition and reverse or inhibit the growth of established tumors will be evaluated.

Biological inhibition of Nrf2 will be done using the SMARTvector Inducible Lentiviral shRNA vectors from GE Dharmacon (Cat #: V3SH11255-01EG4780) according to the manufacturer's instructions. The effectiveness of doxycycline inducible systems has been previously demonstrated in orthotopic GBM models (Puliyappadamba, et al. (2013) Opposing Effect of EGFRWT on EGFRvIII-Mediated NF-kappaB Activation with RIP1 as a Cell Death Switch, Cell Rep 4, 764-775; Lopez-Bertoni, et al. (2016) Epigenetic modulation of a miR-296-5p:HMGA1 axis regulates Sox2 expression and glioblastoma stem cells, Oncogene 35, 4903-4913). Nrf2 will be silenced in Mayo PDX GBM12 and GBM6 or in GL261 EGFR transfected cells and stable populations will be derived using puromycin selection. To confirm silencing we will do Western blot for Nrf2. As a control, shRNA expressing cells will be used. Doxycycline will be used to induce shRNA after detection of tumor on BLI imaging. The groups tested will be (1) control shRNA; (2) Nrf2 shRNA; (3) Nrf2 shRNA+doxycycline; (4) control shRNA+erlotinib; (5) Nrf2 shRNA+erlotinib+doxycycline; and (6) control shRNA+erlotinib+doxycycline. Erlotinib will be administered by oral gavage (50 mg/kg) daily for 4 weeks. A similar experiment will be conducted with afatinib (50 mg/kg) by oral gavage daily for 4 weeks.

Isoniazid (INH), a drug widely used in tuberculosis is a known and potent inhibitor of Nrf2 and is highly CNS penetrable. It was also confirmed that INH blocks erlotinib induced Nrf2 activation (FIG. 4H-I), synergizes with EGFR inhibition in cell survival assays (FIG. 5A and FIG. 5B), and that a combination of erlotinib plus Isoniazid blocks tumor growth in preliminary orthotopic experiments (FIG. 6). INH alone has no effect. INH will be administered by oral gavage at a dose of 25 mg/kg/day for four weeks. The groups will be as follows: (1) Control (DMSO); (2) Erlotinib; (3) INH; and (4) INH+erlotinib. Temozolomide (TMZ) will be used to compare the efficacy compared to EGFR inhibition+INH or other Nrf2 inhibitor. A dose dense TMZ regimen will be used with 25 mg/kg daily for 3 weeks by oral gavage. While INH is a potent inhibitor Nrf2, it is not a specific inhibitor. Thus, ML385, a specific small molecule inhibitor of Nrf2, will also be used. ML385 binds to the Neh1 domain of Nrf2 and has been used in animal studies. Thus, ML385 will be examined, 30 mg/kg, i.p, for 4 weeks. The groups will be as follows: (1) Control (DMSO); (2) ML385; (3) Erlotinib; and (4) ML385+erlotinib. In these experiments an intracranial as well as a subcutaneous group will be examined, since CNS penetration of ML385 is not established. A similar experiment will be conducted with afatinib.

Eight mice per group will be used, based on power analysis. Given the possible, but unresolved, role sex may play in responsiveness to GBM treatment, male and female mice will be randomly allocated to experimental groups. This sample size calculation is based upon tumor volume measured at 4 weeks after drug administration. The comparisons will be made between the control group and each of the experimental groups, respectively. The multiple comparisons will be not be adjusted for this study. Specifications and assumptions for this samples size calculation are: (1) a tumor volume reduction of 50% for the treated group as compared with the control group; (2) a standard deviation of 30% for tumor volume in each of the comparison groups; (3) power of 85% and two-sided type I error rate of 5% and (4) use of two-sample t-test. GBM12 cells will be injected intracranially (1×10⁵) in athymic mice. Once tumors become visible on BLI for intracranial or visible subcutaneously, treatment will be initiated for various groups. Similar experiments will be conducted with Mayo PDX GBM6 and with GL261EGFR cells implanted into C57BL/6 mice as recently described. Total mice: 552.

Injection and Work up of mice. For the orthotopic model mice will be injected with Mayo PDX GBM12 or GBM6 explant cultures or GL261EGFR cells using a stereotactic frame described. All experimental and control cohorts will be examined daily for development of clinical neurological signs including weight loss, hunched posture, lethargy, seizures, etc., in conjunction with periodic BLI imaging. Mice will be sacrificed when clinical examination reveals them to develop neurological symptoms or 12 weeks after treatment. Longitudinal BLI studies will be used to monitor tumor growth using mean light intensity as described. To enhance scientific rigor, BLI will be done by a blinded postdoc who did not undertake the surgery. Histopathological and Kaplan Meier survival analysis analysis will be done as described. Histopathological analysis will be performed with neuropathologist Kimmo Hatanpaa (15 co-author papers). These findings will be evaluated in archival tissue from human GBMs, obtained through collaboration with Dr. Hatanpaa and Dr. Bruce Mickey (Neurosurgeon, 10 co-author studies) and Dr. Kesari (2 co-author papers).

SA3B. Here, the role of xCT/SCL7A11 as a primary executor of the survival effect of Nrf2 activation following EGFR inhibition is examined. xCT/SLC7A11 is a Nrf2 regulated membrane transporter that couples the influx of extracellular cysteine to the efflux of glutamate. xCT/SLC7A11 is prioritized because a number of previous studies have reported that xCT plays an important role in malignant progression and adverse outcome in glioma. xCT is a membrane transporter that couples the influx of extracellular cysteine to the efflux of glutamate. Importantly, the preliminary data indicate that xCT/SLC7A11 is upregulated following EGFR inhibition (FIG. 4A, FIG. 4B, and FIG. 4E) and chemical or biological inhibition of xCT/SLC7A11 can overcome the primary resistance to EGFR inhibition in GBM cells (FIG. 5A-D and FIG. 5F)

Biological inhibition of xCT/SLC7A11 using the SMARTvector Inducible Lentiviral shRNA vectors from GE Dharmacon will be studied as described above in Mayo PDX GBM12 and GBM6. Western blot with xCT/SLC7A11 antibody will be done to confirm silencing. An orthotopic model will be used. Groups will be tested as described above for the shRNA experiment for Nrf2. 8 mice for the experimental and the control groups will be used based on the power calculation for SA3A.

Chemical Inhibition of xCT/SLC7A11. Sulfasalazine inhibits xCT's function by decreasing the supply of Cystine® and has been used successfully in an mouse intracranial model of glioma or seizures. Once intracranial tumors become visible on BLI, the mice will be divided into control, erlotinib alone, Sulfasalazine, or erlotinib+Sulfasalazine. The dose of Sulfasalazine will be 67.5 mg/kg i.p. daily for 3 weeks. These studies will be undertaken with Erastin, a drug shown to specifically inhibit xCT/SLC7A11 in glioma cells. The dose of Erastin will be 20 mg/kg i.p. every day for 4 weeks. Since CNS penetration of Erastin is not established, an intracranial and subcutaneous experiment will be done. For the sc group, tumor size will be measured with calipers twice a week. A similar experiment will be conducted with afatinib. Total mice: 480.

SA3C. Candidates will be prioritized based on the results from the studies above. As an example, a number of GSH related proteins are upregulated. Thus, if a biological role of GSH in mediating resistance to EGFR inhibition is observed, animal experiments will be undertaken with APR-246, a drug that that binds to GSH and depletes its intracellular levels. APR-246 produces synergistic effects with several chemotherapeutic drugs, is effective in PDX models in mice, and is currently being tested in multiple clinical trials. Initial projection of total number of mice (assuming two additional targets): 576.

SA3D. The TCGA and Ivy data indicate that the NRF2 pathway plays an oncogenic role in GBM. However, data is not available for EGFR TKI patients regarding the role of Nrf2 in response to therapy. Without wishing to be bound by theory, it is hypothesized that the basal and/or the TKI-induced Nrf2 levels can predict GBM patient survival when combination therapy with EGFR and Nrf2 inhibition is used. An initial experiment demonstrates that the level of the Nrf2 target gene xCT/SLC7A11 can be used to predict responsiveness to EGFR+Nrf2 inhibition in cell survival assays.

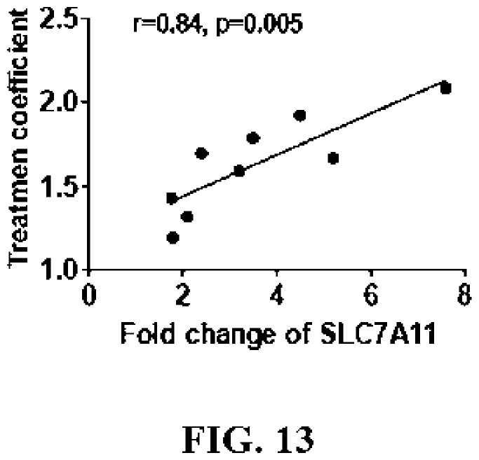

Referring to FIG. 13, the correlation between synergistic effect of EGFR plus Nrf2 inhibition and fold change of xCT/SLC7A11 mRNA levels upon erlotinib treatment. Nine PDX lines were tested. Erlotinib and ML-385 were added to cells concurrently and AlamarBlue cell viability assay was done after 72 h. In a parallel experiment, cells were exposed to erlotinib, for 24 h, and qPCR was performed to measure SLC7A11 mRNA levels. Treatment coefficient=1/Coefficient of Drug Interaction (CDI). CDI was calculated with the equation: CDI=AB/(A×B) (AB, relative cell viability of the combination; A or B, relative cell viability of the single agent groups. Treatment coefficient >1 indicates a synergistic effect. Changes in SLC7A11 mRNA levels show a strong correlation with the combined treatment of erlotinib and Nrf2 inhibitor (ML-385).

To simulate this situation in a mouse experiment, a total of 7 Mayo PDX GBMs will be used in an orthotopic mouse model. The size of 7 was based on a power calculation with effect size of r=0.84, power of 80%, and alpha error of 0.05, under the two-tailed point biserial correlation test. Two experiments will be undertaken: a short-term Erlotinib treatment to measure the basal and the induced NRF2 expression levels; and a long-term study to observe the in vivo combination effects of EGFR and Nrf2 inhibition. In the short-term study, 50 mg/kg/d erlotinib or afatinib by oral gavage will be used to treat mice with intracranial tumors for 0, 2, and 7 days (n=3 per group). GBM tumors will be collected and detected for NRF2 target genes (NQO2, SLC7A11, GSH pathway) by qPCR. The relative gene expression and the induced fold changes will be calculated. Then in the long-term study, 32 nude mice implanted with one strain of PDX will be divided into 4 groups, and daily treated with vehicle, Erlotinib alone, anti-Nrf2 drug alone, and combination therapy. KM plots will be drawn and calculated with median survival days, Hazard-Ratios, and p-values in the Log-Rank test, comparing the combination group with the other 3 groups. Tumor growth will be monitored with MRI imaging as described previously (Gong, et al. (2018) TNF-driven adaptive response mediates resistance to EGFR inhibition in lung cancer, J Clin Invest 128, 2500-2518). Finally, the in vivo relationship of basal/induced NRF2 levels and survival benefits of the combination treatment on these PDXs will be studied by correlation and regression analysis. Total mice: 287. The same type of analysis will be undertaken for post-afatinib treatment GBM samples provided by Dr. Kesari.

Drug toxicities: EGFR and Nrf2/xCT inhibitors do not have overlapping toxicities. Common side effects of EGFR TKIs in humans include skin rash, Gl, and musculoskeletal symptoms. We have given erlotinib or afatinib to mice at 50-100 mg/kg by gavage without significant toxicity. This dose is consistent with previous studies and guidelines for dose conversion between animals and humans. No drug interactions between erlotinib and Isoniazid or erlotinib and sulfasalazine are listed in Micromedex. INH may cause side effects, such as hepatotoxicity, neuropathy, rash or hematologic side effects.

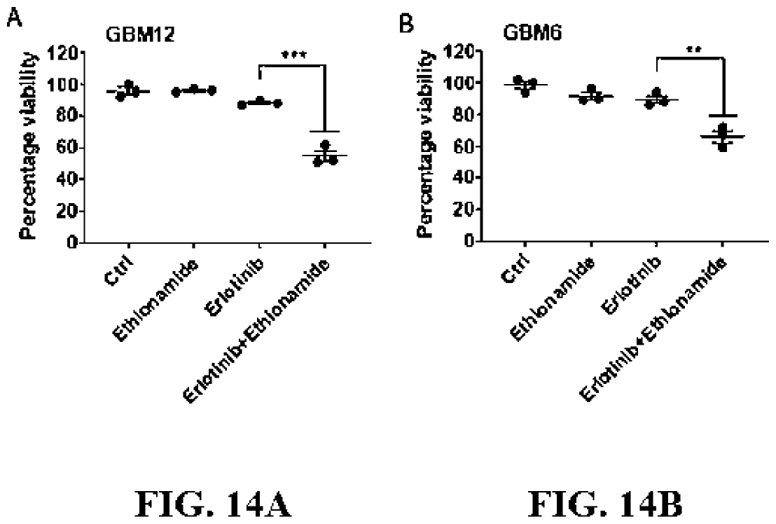

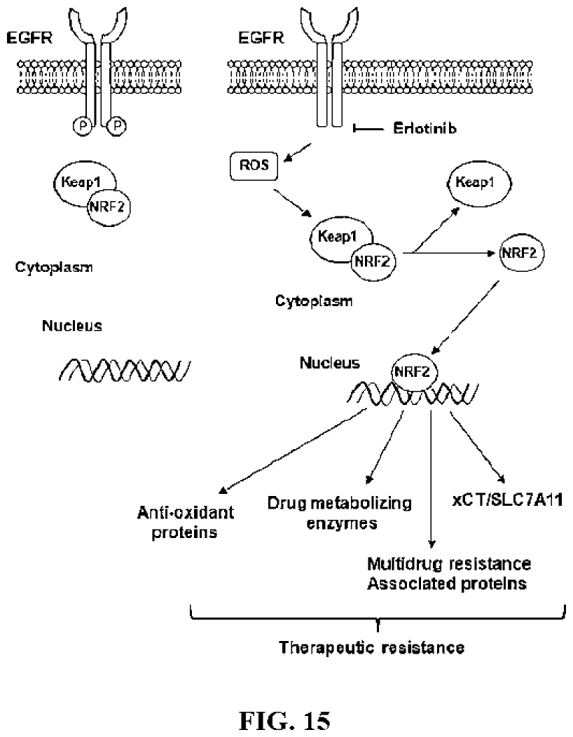

It is anticipated that a combination of EGFR inhibition plus inhibition of Nrf2 or xCT/SLC7A11 will inhibit or reverse the growth of glioma tumors. Previous studies have reported the effective use of erlotinib in intracranial models of glioma in mice (Wykosky, et al. (2015) A urokinase receptor-Bim signaling axis emerges during EGFR inhibitor resistance in mutant EGFR glioblastoma, Cancer Res 75, 394-404; Sarkaria, et al. (2006) Use of an orthotopic xenograft model for assessing the effect of epidermal growth factor receptor amplification on glioblastoma radiation response, Clin Cancer Res 12, 2264-2271). The correlational/PD studies may identify signals in the Nrf2 signaling network that may help to predict responsiveness to erlotinib with or without Nrf2 inhibition. Additional putative targets for inhibition will be selected based on the results of the studies detailed above. An alternative to the use of INH would be ethionamide. Ethionamide is a known Nrf2 inhibitor and preliminary synergizes with EGFR inhibition (FIG. 14).

Referring to FIG. 14A and FIG. 14B, cells were treated by erlotinib (1 μM) and/or ethionamide (1 μM) for 72 hours followed by cell viability assay.

7. Pharmacokinetic Studies

The goal of pharmacokinetic studies is to identify a regimen in mice that mimics human exposure. The studies will be conducted in orthotopic tumor bearing mice to simultaneously examine pharmacodynamic biomarkers. Mice will be examined at 15, 30, 60, 120 mins, then 6, 12, 24, and 48 h post-treatment in groups of three. Mice will be sacrificed at various time points, blood will be collected by cardiac puncture, and tumor and brain tissue also collected. The drugs will be measured in plasma and tumor and brain homogenate using liquid chromatography-tandem mass spectrometry (LC-MS/MS) with an Applied Biosystems/MDS Sciex 4000 QTRAP coupled to a Shimadzu Prominence LC. PK analysis will be performed in the Biochemistry core at UT Southwestern. The drugs will be measured alone or in combinations to determine drug-drug interactions. Total mice: 135.

8. Statistical Analysis

The data will be explored with histograms, box-plots, and scatter plot matrices. For comparison between two groups, a t-test for normally distributed variables will be used. One-way ANOVA models will be used for comparisons of more than two groups, for example, no inhibitor, INH, and INH+erlotinib. For all models, the data will be tested to ensure that the underlying assumptions (i.e., normality and homoscedasticity) are met. If the assumptions are not met, then we will attempt to transform the data in order to meet these assumptions. Standard transformations (e.g., Box-Cox) will be examined. If data transformation is inadequate to meet the analysis assumptions, then rank transformation of the data will be performed. Nonparametric alternatives such as the Wilcoxon signed-rank test, the Wilcoxon rank-sum test, or permutation tests, will be used as appropriate. Bonferroni's adjustment will be used to account for multiple comparisons. The significance level 0.05 will be considered significant. *p<0.05; **p<0.01. ***p<0.001.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method for treating glioma in a subject, the method comprising administering to the subject an effective amount of an agent that modulates epidermal growth factor receptor (EGFR) signaling, or a pharmaceutically acceptable salt thereof, and an agent that modulates Nrf2 signaling, or a pharmaceutically acceptable salt thereof.

2. The method of claim 1, wherein the agent that modulates EGFR signaling is an EGFR inhibitor.

3. The method of claim 2, wherein the EGFR inhibitor is a tyrosine kinase inhibitor.

4. The method of claim 3, wherein the tyrosine kinase inhibitor is erlotinib.

5. The method of claim 2, wherein the EGFR inhibitor is a monoclonal antibody.

6. The method of claim 2, wherein the EGFR inhibitor is selected from erlotinib, afatinib, cetuximab, panitumumab, erlotinib HCl, gefitinib, lapatinib, neratinib, lifirafenib, HER2-inhibitor-1, nazartinib, naquotinib, canertinib, AG-490, CP-724714, Dacomitinib, WZ4002, Sapitinib, CUDC-101, AG-1478, PD153035 HCl, pelitinib, AC480, AEE788, AP261 13-analog, OSI-420, WZ3146, WZ8040, AST-1306, rociletinib, genisten, varlitinib, icotinib, TAK-285, WHI-P154, daphnetin, PD168393, tyrphostin9, CNX-2006, AG-18, AZ5104, osimertinib, CL-387785, olmutinib, AZD3759, poziotinib, vandetanib, and necitumumab.

7. The method of claim 1, wherein the agent that modulates Nrf2 signaling is a Nrf2 inhibitor.

8. The method of claim 7, wherein the Nrf2 inhibitor is selected from isoniazid, ML385, and ethionamide.

9. The method of claim 7, wherein the Nrf2 inhibitor is isoniazid.

10. The method of claim 1, wherein the agent that modulates Nrf2 signaling also modulates xCT/SLC7A11 signaling.

11. The method of claim 10, wherein the agent that modulates Nrf2 signaling also inhibits xCT/SLCA11 signaling.

12. The method of claim 11, wherein the agent that modulates Nrf2 signaling and inhibits xCT/SLCA11 signaling is selected from sulfasalazine and erastin.

13. The method of claim 1, wherein the agent that modulates EGFR signaling is an EGFR inhibitor and wherein the agent that modulates Nrf2 signaling is an Nrf2 inhibitor.

14. The method of claim 1, wherein the agent that modulates EGFR signaling is erlotinib and wherein the agent that modulates Nrf2 signaling is isoniazid.

15. The method of claim 1, wherein the agent that modulates EGFR signaling and the agent that modulates Nrf2 signaling are co-formulated.

16-17. (canceled)

18. The method of claim 1, wherein the agent that modulates EGFR signaling and the agent that modulates Nrf2 signaling are not administered concurrently.

19-27. (canceled)

28. The method of claim 1, wherein the glioma is a malignant glioma.

29-31. (canceled)

32. A method for treating a malignant glioma in a patient in need thereof, said method comprising administering to said patient an effective amount of erlotinib and isoniazid.

33. A pharmaceutical composition comprising:
(a) an agent that modulates EGFR signaling, or a pharmaceutically acceptable salt thereof;
(b) an agent that modulates Nrf2 signaling, or a pharmaceutically acceptable salt thereof; and
(c) a pharmaceutically acceptable carrier,
wherein at least one of the agent that modulates EGFR signaling and the agent that modulates Nrf2 signaling is present in an effective amount.

34. The pharmaceutical composition of claim 33, wherein the agent that modulates EGFR signaling is erlotinib and wherein the agent that modulates Nrf2 signaling is isoniazid.

35-53. (canceled)
